(19) United States
(12) Patent Application Publication
(10) Pub. No.: US 2015/0284444 A1
(43) Pub. Date: Oct. 8, 2015

(54) PHARMACEUTICAL COMPOSITION FOR CANCER TREATMENT INCLUDING FUSION PROTEIN

(71) Applicant: Masami Watanabe, (US)

(72) Inventors: Masami Watanabe, Okayama (JP); Hiromi Kumon, Okayama (JP); Yasutomo Nasu, Okayama (JP)

(21) Appl. No.: 14/379,104

(22) PCT Filed: Feb. 15, 2013
(86) PCT No.: PCT/JP2013/053613

§ 371 Date: Jun. 17, 2015

(30) Foreign Application Priority Data
Feb. 16, 2012 (JP) 2012-032073
Jun. 1, 2012 (JP) 2012-126467

(51) Int. Cl. C07K 14/535; C12N 9/64; C12N 9/16; C12N 9/48; A61K 39/00; C07K 14/47; A61K 48/00; A61K 38/20; A61K 38/19; C07K 14/54; C07K 14/705

(52) U.S. Cl. CPC C07K 14/535; C07K 14/54; C07K 14/5406; C07K 14/5418; C12N 9/6424; C12N 9/16; C12N 9/48; C07K 14/705; C07K 14/70596; C07K 14/47; A61K 48/00; A61K 38/2013; A61K 38/2026; A61K 38/2046; A61K 38/193; A61K 39/0011; C07K 2319/00; C12Y 301/03002; C12Y 304/21077; C12Y 304/17021

(57) ABSTRACT

An object of the present invention is to use fusion proteins of cancer-specific antigens and cytokines as a preventive or therapeutic agent for cancer. The present invention provides a pharmaceutical composition for the prevention or treatment of a cancer, comprising as active ingredients fusion proteins each comprising a cancer-specific antigen with a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

### TECHNICAL FIELD

The present invention relates to a pharmaceutical composition for the treatment of a cancer.

### BACKGROUND ART

“Cancer vaccines” are currently receiving attention as a novel therapeutic strategy for cancers. Various approaches such as dendritic cell therapy or peptide vaccines have been studied. A dendritic cell vaccine Sipuleucel-T (Provenge®) for a prostate cancer antigen PAP (prostatic acid phosphatase) was approved by US FDA in April 2011 (see Non Patent Literature 1). This drug is a cellular medicine that is prepared by: collecting peripheral blood mononuclear cells (PBMCs) from a patient; adding thereto PAP-hGMCSF fusion proteins (produced in insect cells); and culturing the cells for approximately 2 days. This cellular medicine is administered to the same patient through intravenous injection.

IL2, IL4, IL7, and GMCSF have been reported as cytokines that promote differentiation into dendritic cells and thereby activate antitumor immunity (see Non Patent Literatures 2 and 3). Non Patent Literature 2 discloses that IL2, IL4, IL7, and GMCSF act on human peripheral blood mononuclear cells (PBMCs) to promote their differentiation into dendritic cells and thereby activate antitumor immunity. Non Patent Literature 3 discloses that IL2, IL4, and IL7 act on human peripheral blood mononuclear cells (PBMCs) to promote their differentiation into lymphocytes and thereby activate antitumor immunity.

Unfortunately, the therapeutic effects of Sipuleucel-T improve a survival period only by 4.1 months. Thus, the development of a therapy having stronger therapeutic effects is an urgent issue. The cytokines IL2, IL4, IL7, and GMCSF disclosed in Non Patent Literatures 2 and 3 had previously been expected to have antitumor effects. In actuality, each cytokine has not been reported to have effective therapeutic effects in the treatment of a cancer such as prostate cancer. In addition, each cytokine has not been clinically used in the treatment of a cancer such as prostate cancer.

### CITATION LIST

### Non Patent Literature

Non Patent Literature 1: Kantoff P W et al., N Engl J Med. 2010 Jul. 29; 363 (5): 411-22
Non Patent Literature 2: Zou G M et al., Eur Cytokine Netw. 2002 April-June; 13 (2): 186-99
Non Patent Literature 3: Alderson M R et al. J Exp Med. 1990 Aug. 1; 172 (2): 577-87

### SUMMARY OF INVENTION

### Technical Problem

An object of the present invention is to use fusion proteins of cancer-specific antigens and cytokines as a preventive or therapeutic agent for a cancer. Particularly, an object of the present invention is to provide a preventive or therapeutic agent for a cancer, comprising as active ingredients fusion proteins each comprising a cancer-specific antigen which is prostate-specific antigen (PSA), prostatic acid phosphatase (PAP), prostate-specific membrane antigen (PSMA), a melanoma-associated antigen 4 (MAGEA4), CD147, or carcinoembryonic antigen (CEA) with a cytokine which is human IL2 (hIL2), human IL4 (hIL4), human IL7 (hIL7), human GMCSF (hGMCSF), mouse IL4 (mIL4), or mouse GMCSF (mGMCSF).

### Solution to Problem

The present inventors have conducted diligent studies on the development of a cancer therapy by promoting in vivo anticancer activity targeting prostate cancer. The present inventors have examined the effects of fusion proteins of human PSA or human PAP with human IL2 (hIL2), human IL4 (hIL4), human IL7 (hIL7), human GMCSF (hGMCSF), mouse IL4 (mIL4), or mouse GMCSF (mGMCSF) on in vivo antitumor immunity activity.

As a result, the present inventors have found that: fusion proteins of human PSA or PAP with mouse-derived mGMCSF or mIL4 exhibit anticancer effects in treatment experiments using prostate cancer mouse models; and the fusion proteins of human PSA or PAP with mouse-derived mGMCSF or mIL4 have the ability to induce the differentiation of mouse-derived peripheral blood monocytes into dendritic cells. This indicates that the fusion proteins of human PSA or PAP with mouse-derived mGMCSF or mIL4 enhance the effects of mouse dendritic cells presenting the human antigen PSA or PAP in vivo in prostate cancer mouse models and induce antitumor effects on cancer cells expressing the antigen. The present inventors have further found that fusion proteins of human PSA or PAP with human-derived hGMCSF or hIL4 have the ability to induce the differentiation of human-derived peripheral blood monocytes into dendritic cells, and have found that, as confirmed in mouse models of human prostate cancer, the fusion proteins of human PSA or PAP with human-derived hGMCSF or hIL4 can also induce anticancer therapeutic effects based on immunity against the human PSA or PAP in human prostate cancer patients. In addition, the present inventors have found that, similarly, fusion proteins of human PSA or PAP with hIL2 or hIL7 can also induce anticancer therapeutic effects based on immunity against the human PSA or PAP in human prostate cancer patients. The present inventors have further prepared fusion proteins of PSMA, MAGEA4, CD147, or CEA with cytokines and found that these fusion proteins can induce anticancer therapeutic effects based on immunity against the antigen PSMA, MAGEA4, CD147, or CEA in cancer patients. On the basis of these findings, the present invention has been completed.

Specifically, the present invention is as follows:

[1] A pharmaceutical composition for the prevention or treatment of a cancer, comprising as active ingredients one or two or more fusion proteins each comprising a cancer-specific antigen and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

[2] The pharmaceutical composition for the prevention or treatment of a cancer according to [1], wherein the cancer-specific antigen is prostate-specific antigen (PSA) or prostatic acid phosphatase (PAP), and the cancer to be prevented or treated is prostate cancer.

[3] The pharmaceutical composition for the prevention or treatment of a cancer according to [1], wherein the cancer-specific antigen is prostate-specific membrane antigen (PSMA), and the cancer to be prevented or treated is prostate cancer.

[4] The pharmaceutical composition for the prevention or treatment of a cancer according to [1], wherein the cancer-specific antigen is a cancer-specific antigen selected from the group consisting of MAGEA4, CD147, and carcinoembryonic antigen (CEA).

[5] The pharmaceutical composition for the prevention or treatment of a cancer according to [4], wherein the cancer to be prevented or treated is selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma.

[6] A method for preparing an immunocompetent cell having antitumor immunity activity, comprising culturing a cell capable of differentiating into an immunocompetent cell in vitro in the presence of one or two or more fusion proteins each comprising a cancer-specific antigen and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

[7] The method for preparing an immunocompetent cell having antitumor immunity activity according to [6], wherein the cancer-specific antigen is prostate-specific antigen (PSA) or prostatic acid phosphatase (PAP), and the cancer to be prevented or treated is prostate cancer.

[8] The method for preparing an immunocompetent cell having antitumor immunity activity according to [6], wherein the cancer-specific antigen is prostate-specific membrane antigen (PSMA), and the cancer to be prevented or treated is prostate cancer.

[9] The method for preparing an immunocompetent cell having antitumor immunity activity according to [6], wherein the cancer-specific antigen is a cancer-specific antigen selected from the group consisting of MAGEA4, CD147, and carcinoembryonic antigen (CEA).

[10] The method for preparing an immunocompetent cell having antitumor immunity activity according to [9], wherein the cancer to be prevented or treated is selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma.

[11] The method for preparing an immunocompetent cell having antitumor immunity activity according to any of [6] to [10], wherein the cell capable of differentiating into an immunocompetent cell is a mononuclear cell obtained from peripheral blood, bone marrow fluid, or umbilical cord blood.

[12] The method for preparing an immunocompetent cell having antitumor immunity activity according to any of [6] to [10], wherein the cell capable of differentiating into an immunocompetent cell is a stem cell.

[13] The method for preparing an immunocompetent cell according to any of [6] to [12], wherein the immunocompetent cell is an antigen-presenting cell or an activated lymphocyte.

[14] A pharmaceutical composition for the prevention or treatment of a cancer, comprising an immunocompetent cell prepared by a method according to any of [6] to [13].

[15] A DNA construct in which any of DNAs encoding 48 types of fusion proteins represented by PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, PAP-mGMCSF, PSMA-hIL2, PSMA-hIL4, PSMA-hIL7, PSMA-hGMCSF, PSMA-mIL4, PSMA-mGMCSF, MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF, CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4 and CEA2-mGMCSF is inserted in a gene insert moiety in any of three constructs having the following structures:

[16] A vector comprising a DNA construct according to [15].

[17] A preparation for the treatment of a cancer, comprising a vector according to [16].

[18] The preparation for the treatment of a cancer according to [17], wherein the preparation is for the treatment of a cancer selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma.

[19] A pharmaceutical composition for the prevention or treatment of a disease involving CD147, comprising as active ingredients one or two or more fusion proteins each comprising CD147 and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

[20] A method for preparing a cell usable in the prevention or treatment of a disease involving CD147, comprising culturing a cell capable of differentiating into an immunocompetent cell in vitro in the presence of one or two or more fusion proteins each comprising CD147 and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

[21] The pharmaceutical composition for the prevention or treatment according to [19] or the method for preparing a cell usable in the prevention or treatment according to [20], wherein the disease involving CD147 is selected from the group consisting of a lung disease, a malignant disease, an immunity-related disease, a cardiovascular disease, a nervous system disease, a fibrosis, and an infection.

The present specification encompasses the contents described in the specifications and/or drawings of Japanese Patent Application Nos. 2012-032073 and 2012-126467 on which the priority of the present application is based.

### Advantageous Effects of Invention

Fusion proteins of cancer-specific antigens such as PSA, PAP, PSMA, MAGEA4, CD147, or CEA with hIL2, hIL4, hIL7, hGMCSF, mIL4, or mGMCSF can be used in the prevention or treatment of a cancer. Such fusion proteins comprising PSA, PAP, or PSMA as a cancer-specific antigen can be used in the specific prevention or treatment of prostate cancer. Alternatively, such fusion proteins comprising MAGEA4, CD147, or CEA can be used in the prevention or treatment of a wide range of cancer types including large intestine cancer, bladder cancer, lung cancer, and stomach cancer. These fusion proteins can enhance the antitumor immunity activity (antitumor activity) of immunocompetent cells in vivo or ex vivo. The fusion proteins can also enhance the antitumor immunity activity of dendritic cells in vivo through direct administration to the organism. Alternatively, the fusion proteins may be used in ex vivo cell therapy which involves: culturing monocytes or lymphocytes (such as cytotoxic lymphocytes, helper T lymphocytes, or B lymphocytes) isolated from an organism, in the presence of the fusion proteins to prepare ex vivo antigen-presenting cells or activated lymphocytes having antitumor immunity activity; and bringing these immunocompetent cells back to the organism. Moreover, ex vivo treatment can be achieved using stem cells capable of differentiating into immunocompetent cells by the fusion proteins of the present invention.

The fusion proteins may be produced by use of a system using an expression cassette that comprises a DNA construct at least comprising a gene encoding each protein to be expressed (gene to be expressed) and a poly-A addition sequence downstream of a first promoter and has a structure where an enhancer or a second promoter is further linked downstream of the construct. In such a case, the fusion proteins can be produced in large amounts in a short period. Particularly, use of this system in human cells allows the fusion proteins safe for humans to be efficiently prepared in large amounts.

### BRIEF DESCRIPTION OF DRAWINGS

FIG. 1 is a diagram showing the structure of an expression cassette for use in the preparation of fusion proteins of cancer-specific antigens and cytokines (part 1).

FIG. 2 is a diagram showing the structure of an expression cassette for use in the preparation of fusion proteins of cancer-specific antigens and cytokines (part 2).

FIG. 3-1 is a diagram showing the structure of an expression cassette for use in the preparation of fusion proteins of cancer-specific antigens and cytokines (part 3).

FIG. 3-2 is a diagram showing the structure of an expression cassette for use in the preparation of fusion proteins of cancer-specific antigens and cytokines (part 4).

FIG. 4-1 is a diagram showing the structure and sequence of an expression cassette for use in the preparation of PSA-cytokine fusion proteins.

FIG. 4-2 is a diagram showing the structure and sequence of an expression cassette for use in the preparation of PSA-cytokine fusion proteins (a sequel to FIG. 4-1).

FIG. 5-1 is a diagram showing the structure and sequence of an expression cassette for use in the preparation of PAP-cytokine fusion proteins.

FIG. 5-2 is a diagram showing the structure and sequence of an expression cassette for use in the preparation of PAP-cytokine fusion proteins (a sequel to FIG. 5-1).

FIG. 6-1 is a diagram showing the nucleotide sequences of cytokines (human IL2, human GMCSF, and human IL7) used in the production of fusion proteins of PSA or PAP with any of the cytokines.

FIG. 6-2 is a diagram showing the nucleotide sequences of cytokines (human IL4, mouse IL4, and mouse GMCSF) used in the production of fusion proteins of PSA or PAP with any of the cytokines.

FIG. 6-3 is a diagram showing the whole nucleotide sequence of a pIDT-SMART vector.

FIG. 7-1 is a diagram showing results of electrophoresing and CBB-staining prepared fusion proteins.

FIG. 7-2 is a diagram showing results of electrophoresing and CBB-staining fusion proteins obtained by affinity purification.

FIG. 7-3 is a diagram showing the concentrations of fusion proteins in obtained fusion protein solutions.

FIG. 8 is a diagram showing a time-dependent rise in serum PSA or PAP levels and tumor formation in mouse models of human prostate cancer. FIG. 8a shows the results about PSA-RM9 cell-transplanted mice. FIG. 8b shows the results about PAP-RM9 cell-transplanted mice.

FIG. 9-1 is a diagram showing the therapeutic effects of fusion proteins (intraperitoneally administered) on mouse models of human prostate cancer.

FIG. 9-2 is a diagram showing the therapeutic effects of fusion proteins (administered from tail veins) on mouse models of human prostate cancer.

FIG. 10-1 is a diagram showing the morphology of human dendritic cells induced from human PBMCs 7 days after addition of commercially available hGMCSF and hIL4 proteins thereto.

FIG. 10-2 is a diagram showing the rate of emergence of dendritic cells induced from mouse peripheral blood mononuclear cells (PBMCs) by the addition of PSA-mGMCSF and PSA-mIL4 in combination or PAP-mGMCSF and PAP-mIL4 in combination.

FIG. 10-3 is a diagram showing the rate of emergence of dendritic cells induced from human peripheral blood mononuclear cells (PBMCs) by the addition of PSA-hGMCSF and PSA-hIL4 in combination or PAP-hGMCSF and PAP-hIL4 in combination.

FIG. 11 is a diagram showing results of analyzing the cell growth effects of purified PSA-hGMCSF and PAP-hGMCSF on TF-1 cells by MTT assay.

FIG. 12 is a diagram showing results of purifying (concentrating), electrophoresing, and CBB-staining PSA- or PAP-containing fusion proteins. FIG. 12a shows the results about PSA-hGMCSF, PAP-hGMCSF, PSA-hIL2, and PAP-hIL2. FIG. 12b shows the results about PSA-hIL4, PAP-hIL4, PSA-hIL7, and PAP-hIL7.

FIG. 13-1 is a diagram showing the structure and sequence of an expression cassette for use in the preparation of PSMA-cytokine fusion proteins.

FIG. 13-2 is a diagram showing the structure and sequence of an expression cassette for use in the preparation of PSMA-cytokine fusion proteins (a sequel to FIG. 13-1).

FIG. 13-3 is a diagram showing the structure and sequence of an expression cassette for use in the preparation of PSMA-cytokine fusion proteins (a sequel to FIG. 13-2).

FIG. 14-1 is a diagram showing results of purifying, electrophoresing, and CBB-staining a PSMA-hGMCSF fusion protein.

FIG. 14-2 is a diagram showing the concentration of a PSMA-hGMCSF fusion protein in an obtained PSMA-hGMCSF fusion protein solution.

FIG. 15 is a diagram showing results of analyzing the cell growth effects of purified PSMA-hGMCSF on TF-1 cells by MTT assay (also including results about PSA-hGMCSF and PAP-hGMCSF).

FIG. 16-1 is a diagram showing the structure and sequence of an expression cassette for use in the preparation of MAGEA4-cytokine fusion proteins.

FIG. 16-2 is a diagram showing the structure and sequence of an expression cassette for use in the preparation of MAGEA4-cytokine fusion proteins (a sequel to FIG. 16-1).

FIG. 16-3 is a diagram showing the structure and sequence of an expression cassette for use in the preparation of MAGEA4-cytokine fusion proteins (a sequel to FIG. 16-2).

FIG. 17-1 is a diagram showing the structure and sequence of an expression cassette for use in the preparation of CD147-cytokine fusion proteins.

FIG. 17-2 is a diagram showing the structure and sequence of an expression cassette for use in the preparation of CD147-cytokine fusion proteins (a sequel to FIG. 17-1).

FIG. 18-1 is a diagram showing results of purifying, electrophoresing, and CBB-staining MAGEA4- or CD147-cytokine fusion proteins.

FIG. 18-2 is a diagram showing the concentrations of MAGEA4- or CD147-cytokine fusion proteins in obtained fusion protein solutions.

FIG. 19-1 is a diagram showing the structure and sequence of an expression cassette for use in the preparation of CEA-cytokine fusion proteins.

FIG. 19-2 is a diagram showing the structure and sequence of an expression cassette for use in the preparation of CEA-cytokine fusion proteins (a sequel to FIG. 19-1).

FIG. 19-3 is a diagram showing the structure and sequence of an expression cassette for use in the preparation of CEA-cytokine fusion proteins (a sequel to FIG. 19-2).

FIG. 20 is a diagram showing the amino acid sequences of CEA and NCA.

FIG. 21 is a diagram showing the purities of purified fusion proteins. FIG. 21A shows the results about fusion proteins of CEA1 and each cytokine before and after purification. FIG. 21B shows the results about fusion proteins of CEA2 and each cytokine before and after purification. FIG. 21C shows the results about fusion proteins of PSMA and each cytokine after purification.

FIG. 22 is a diagram showing the concentrations of various fusion proteins after purification.

FIG. 23-1 is a diagram showing the induction of dendritic cells using PSA-hGMCSF in combination with various fusion proteins.

FIG. 23-2 is a diagram showing the induction of dendritic cells using PAP-hGMCSF in combination with various fusion proteins.

FIG. 23-3 is a diagram showing the induction of dendritic cells using PSMA-hGMCSF in combination with various fusion proteins.

FIG. 23-4 is a diagram showing the induction of dendritic cells using CD147-hGMCSF in combination with various fusion proteins.

FIG. 23-5 is a diagram showing the induction of dendritic cells using MAGEA4-hGMCSF in combination with various fusion proteins.

FIG. 23-6 is a diagram showing the induction of dendritic cells using CEA1-hGMCSF in combination with various fusion proteins.

FIG. 23-7 is a diagram showing the induction of dendritic cells using CEA2-hGMCSF in combination with various fusion proteins.

FIG. 24 is a diagram showing results of analyzing the induction of dendritic cells using various fusion proteins and combinations of fusion proteins by flow cytometry.

FIG. 25-1 is a diagram showing results of analyzing the induction of cytotoxic T lymphocytes (CD8-positive) using various fusion proteins and combinations of fusion proteins by flow cytometry.

FIG. 25-2 is a diagram showing results of analyzing the induction of helper T lymphocytes (CD4-positive) using various fusion proteins and combinations of fusion proteins by flow cytometry.

FIG. 25-3 is a diagram showing results of analyzing the induction of B lymphocytes (CD19-positive) using various fusion proteins and combinations of fusion proteins by flow cytometry.

FIG. 26 is a diagram showing the protocol of an experiment showing the effects of fusion proteins on large intestine cancer.

FIG. 27 is a diagram showing the effects of fusion proteins on large intestine cancer.

FIG. 28 is a diagram showing the protocol of an experiment showing the effects of fusion proteins on bladder cancer.

FIG. 29 is a diagram showing the effects of fusion proteins on bladder cancer.

FIG. 30 is a diagram showing the protocol of an experiment showing the effects of fusion proteins on lung cancer.

FIG. 31 is a diagram showing the effects of fusion proteins on lung cancer.

FIG. 32 is a diagram showing the protocol of an experiment showing the effects of fusion proteins on stomach cancer.

### DESCRIPTION OF EMBODIMENTS

Hereinafter, the present invention will be described in detail.

The present invention provides a pharmaceutical composition for the prevention or treatment of a cancer, comprising as active ingredients fusion proteins each comprising a cancer-specific antigen or an antigen that is expressed at an increased level in cancer cells compared with normal cells, and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF). These fusion proteins each comprising a cancer-specific antigen or an antigen that is expressed at an increased level in cancer cells compared with normal cells, and a cytokine have the original functions of the cytokine fused with the cancer-specific antigen or the antigen that is expressed at an increased level in cancer cells compared with normal cells. In the present invention, the cancer-specific antigen also includes the antigen that is expressed at an increased level in cancer cells compared with normal cells.

Examples of the cancer-specific antigen used in the present invention include: human prostate cancer-specific antigen (PSA), human prostatic acid phosphatase (PAP), and prostate-specific membrane antigen (PSMA) for prostate cancer; carcinoembryonic antigen (CEA) for large intestine cancer and digestive organ cancer; HER2/neu and malignant melanoma for breast cancer; antigens (MAGEs) belonging to the MAGE (melanoma antigen) gene family, such as MAGEA4, for other various cancers; WT1 peptide for leukemia and various cancers; glypican 3 (GPC3) for hepatocellular cancer; and MUC1 (Mucin 1), hTERT (human telomerase reverse transcriptase), AKAP-4 (A-kinase anchor protein-4), Survivin (baculoviral inhibitor of apoptosis repeat-containing 5), NY-ESO-1 (New York esophageal squamous cell carcinoma 1), and CD147 for various cancers.

Hereinafter, the present invention will be described by taking PSA, PAP, PSMA, MAGEA4, CD147, and CEA as examples. Fusion proteins of other cancer-specific antigens and cytokines can be prepared on the basis of the description about PSA, PAP, PSMA, MAGEA4, CD147, and CEA. These fusion proteins can be used in the treatment of a cancer.

PSA is a single-chain glycoprotein with a molecular weight of approximately 34,000 that is specifically found in prostate tissues. PSA exhibits an elevated serum level in prostate cancer, benign prostatic hypertrophy, prostatitis, and other prostatic diseases and is strongly expressed, particularly, in prostate cancer. PAP is a phosphatase that is found in the prostate, erythrocytes, platelets, leukocytes, the spleen, the liver, the kidney, and bones. This enzyme hydrolyzes phosphoester in an acidic solution. PAP is a glycoprotein that is produced by prostate epithelial cells and is contained in a prostate tissue-specific fraction. PAP is strongly expressed, particularly, in prostate cancer.

The prostate-specific membrane antigen (PSMA) protein is specifically expressed in the prostate epithelium and overexpressed in prostate cancer. Its enzyme activity is also increased in prostate cancer compared with normal tissues and benign prostatic hypertrophy tissues. Its expression has been observed in many cases of refractory prostate cancer that is at an advanced stage resistant to endocrine therapy.

The melanoma antigen (MAGE) gene family is a gene family encoding tumor regression antigens that are specifically recognized by cytotoxic T cells. The MAGE genes constitute a multigene family involving 12 genes: MAGE-1 to MAGE-12. MAGEA4 is included in this family. The expression of this family is not seen in normal tissues except for testis and placenta or in areas other than the skin during wound healing and is expressed with high frequency in a wide range of cancer types. Specifically, this protein is overexpressed in melanoma, breast cancer, lung cancer, stomach cancer, bladder cancer, hepatocellular cancer, esophagus cancer, brain tumor, blood cancer, etc.

The CD147 protein, also called Bisigin or extracellular matrix metalloproteinase inducer (EMMPRIN), is a 27 kDa glycoprotein. CD147 is a molecule that enhances collagenase activity in cancer cells and participates in the cell adhesion of the cancer cells. CD147 is highly expressed in many types of cancer cells and strongly involved in cancer infiltration, metastasis, and progression by inducing matrix metalloproteinase (MMP)-1, -2, or -3 or the like in neighboring mesenchymal cells. Examples of the cancer types overexpressing CD147 include bladder cancer, breast cancer, lung cancer, mouth cancer, esophagus cancer, skin cancer, malignant lymphoma, glioma, ovary cancer, melanoma, and hepatocellular cancer.

The carcinoembryonic antigen (CEA) protein is a cell adhesion factor-related glycoprotein serving as a tumor marker. This protein is overexpressed in a wide range of cancer types including large intestine cancer, rectal cancer, thyroid gland cancer, esophagus cancer, stomach cancer, breast cancer, gallbladder cancer, bile duct cancer, lung cancer, pancreatic cancer, uterine cervix cancer, ovary cancer, bladder cancer, and medullary thyroid cancer. The full-length sequence or partial sequence of CEA may be used. Examples of the partial sequence include CEA1 having the amino acid sequence represented by SEQ ID NO: 17 and CEA2 having the amino acid sequence represented by SEQ ID NO: 19. CEA also constitutes a CEA family together with other proteins having high amino acid sequence identity, including NCA (non-specific cross-reacting antigen), PSG (pregnancy-specific glycoprotein), and the like. In the present invention, these proteins belonging to the CEA family can also be used. The full-length sequences of these proteins or their fragments corresponding to the CEA1 or the CEA2 may be used. In the present invention, the term CEA includes both CEA1 and CEA2. Use of CEA permits the treatment of a cancer or the immunotherapy of other diseases targeting any of the proteins belonging to the CEA family.

The cancer-specific antigen for use in the preparation of fusion proteins may have a full-length amino acid sequence or, in the case of a transmembrane protein, may have the amino acid sequence of an extracellular region. For example, PSMA and CD147 are transmembrane proteins. A fusion protein of PSMA or CD147 with a cytokine may contain the extracellular region of PSMA or CD147 fused with the cytokine.

Examples of the fusion proteins contained as active ingredients in the pharmaceutical composition for the prevention or treatment of a cancer according to the present invention can include fusion proteins each comprising a cancer-specific antigen selected from the group consisting of human prostate cancer-specific antigen (PSA), human prostatic acid phosphatase (PAP), prostate-specific membrane antigen (PSMA), MAGEA4, CD147, and carcinoembryonic antigen (CEA), and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF). In the present invention, for example, the fusion protein of PSA or PAP with each of these cytokines is referred to as PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, or PAP-mGMCSF. Also, the fusion protein of PSMA, MAGEA4, CD147, or CEA with each of these cytokines is referred to as PSMA-hIL2, PSMA-hIL4, PSMA-hIL7, PSMA-hGMCSF, PSMA-mIL4, PSMA-mGMCSF, MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF.

The present invention encompasses these 36 types of fusion proteins and a pharmaceutical composition comprising these fusion proteins. CEA1 or CEA2 may be used as CEA. The fusion protein thereof with each of the cytokines is referred to as CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4, CEA2-mGMCSF. The present invention also encompasses these fusion proteins and a pharmaceutical composition comprising these fusion proteins. The present invention encompasses 48 types of fusion proteins including the fusion proteins of CEA1 or CEA2.

In order to obtain these fusion proteins, a gene encoding PSA, PAP, PSMA, MAGEA4, CD147, or CEA and a gene encoding the cytokine of interest can be linked in flame and expressed. The genes can be linked by a conventional gene recombination approach. This linking can be carried out by the introduction of appropriate restriction sites. It is required that no stop codon should exist between the genes to be fused. The distance between the genes to be fused is not limited and may involve a linker. The cancer-specific antigen PSA, PAP, PSMA, MAGEA4, CD147, or CEA may be fused to the N terminus or C-terminus of the amino acid sequence of the cytokine.

The fusion gene thus prepared is incorporated into an available appropriate expression vector and expressed, and the fusion protein of interest can be recovered and purified. Alternatively, a cell-free system may be used for this expression.

Any vector such as a plasmid, a phage, or a virus can be used as long as the vector is replicable in host cells. The vector contains a replication origin, a selection marker, and a promoter and may optionally contain an enhancer, a terminator, a ribosomal binding site, a polyadenylation signal, and the like.

The DNA can be transferred to the vector by a method known in the art. Desirably, the vector contains a polylinker having various restriction sites in its internal region or contains a single restriction site. A particular restriction site in the vector is cleaved with a specific restriction enzyme, and the DNA can be inserted into the cleavage site. The expression vector comprising the fusion gene can be used in the transformation of suitable host cells, which are then allowed to express and produce a fusion protein encoded by the fusion gene.

Examples of the host cells include cells of bacteria such as E. coli, Streptomyces, and Bacillus subtilis, fungal cells, baker's yeast, yeast cells, insect cells, and mammalian cells.

The transformation can be carried out by a method known in the art such as calcium chloride-, calcium phosphate-, or DEAE-dextran-mediated transfection, electroporation, or lipofection.

The obtained recombinant fusion protein can be separated or purified by any of various separation or purification methods. For example, ammonium sulfate precipitation, gel filtration, ion-exchange chromatography, and affinity chromatography can be used alone or in appropriate combination. In this respect, the expression product expressed as a fusion protein with a protein or peptide such as GST may be purified by use of the properties of the protein or the peptide fused with the protein of interest. For example, the expressed fusion protein with GST can be efficiently purified by affinity chromatography using a column composed of a glutathione-bound support, because GST has affinity for glutathione. Alternatively, the expressed fusion protein with a histidine tag can be purified using a chelate column, because such a protein having the histidine tag binds to the chelate column.

The present inventors have developed a gene expression system for enhancing gene expression. The fusion protein is preferably prepared using this gene expression system. The gene expression system is described in WO2011/062298. Each fusion protein of the present invention can be produced according to the description of the patent literature.

Specifically, the fusion protein can be expressed using the gene expression system.

The gene expression system employs an expression cassette that comprises a DNA construct at least comprising a gene encoding each protein to be expressed (gene to be expressed) and a poly-A addition sequence downstream of a first promoter and has a structure where an enhancer or a second promoter is further linked downstream of the construct. The gene to be expressed is inserted to a multicloning site in the expression cassette for expression of the gene. In this case, the gene to be expressed can be inserted to the multicloning site (insertion site) by use of a sequence that is recognized by a restriction enzyme. For this purpose, a DNA in which the DNA encoding the cancer-specific antigen such as PSA, PAP, PSMA, MAGEA4, CD147, or CEA is linked to the DAN encoding the cytokine may be inserted to the multicloning site. Alternatively, the DNA encoding the cancer-specific antigen such as PSA, PAP, PSMA, MAGEA4, CD147, or CEA may be incorporated in advance upstream or downstream of the multicloning site, and only the DNA encoding the cytokine to prepare the fusion protein with this cancer-specific antigen can be inserted to the multicloning site.

More specifically, the expression cassette mentioned above comprises (i) a DNA construct comprising a first promoter, a gene to be expressed, and a poly-A addition sequence linked in this order and (ii) an enhancer or an enhancer with UAS linked upstream thereof, in the order of (i) and (ii), and has a structure where the enhancer or the enhancer with UAS linked upstream thereof is linked immediately downstream of the poly-A addition sequence. Use of this expression cassette enhances protein expression from the gene compared with an expression cassette having an enhancer inserted upstream of the first promoter. In a preferred structure, the gene to be expressed is flanked by the first promoter and the enhancer without having other mechanisms for gene expression downstream of the linked enhancer. The promoter used is not limited and is preferably a CMV i promoter, an SV40 promoter, an hTERT promoter, a β actin promoter, or a CAG promoter. A core promoter moiety consisting of the minimum sequence having promoter activity may be used as the promoter.

The poly-A addition sequence (polyadenylation sequence: poly-A) is not limited by its origin. Examples thereof include growth hormone gene-derived poly-A addition sequences, for example, a bovine growth hormone gene-derived poly-A addition sequence and a human growth hormone gene-derived poly-A addition sequence, an SV40 virus-derived poly-A addition sequence, and a human or rabbit β globin gene-derived poly-A addition sequence. The poly-A addition sequence contained in the expression cassette enhances transcription efficiency.

The enhancer to be linked downstream of the poly-A addition sequence is not limited. Preferably, a CMV enhancer, an SV40 enhancer, an hTERT (telomerase reverse transcriptase) enhancer, or the like can be used. One type of enhancer may be used, or two or more identical or different enhancers may be used in combination. One example thereof includes a linkage of an hTERT enhancer, an SV40 enhancer, and a CMV enhancer in this order. UAS may be linked immediately upstream of the enhancer. UAS refers to a GAL4 gene-binding region and enhances protein expression as a result of subsequent insertion of the GAL4 gene.

A plurality of enhancers, for example, 1 to 4 enhancers, may be further linked upstream of the DNA construct comprising a DNA encoding each protein to be expressed and a poly-A addition sequence. The enhancer(s) to be linked upstream thereof is not limited and is preferably a CMV enhancer. Examples thereof include a linkage of four CMV enhancers, i.e., 4×CMV enhancer. The enhancer inserted immediately downstream of the DNA construct comprising “promoter—gene to be expressed—poly-A addition sequence” permits strong protein expression from the gene to be expressed compared with a conventional general gene expression system.

RU5′ may be further linked immediately upstream of the DNA encoding the protein to be expressed. The term “immediately upstream” refers to direct linkage without being mediated by any of other elements having particular functions and however, accepts the intervention of a short sequence as a linker. RU5′ refers to an HTLV-derived LTR element that enhances protein expression (Mol. Cell. Biol., Vol. 8 (1), p. 466-472, 1988).

SV40-ori may be further linked most upstream of the expression cassette. SV40-ori refers to an SV40 gene-binding region and enhances protein expression as a result of subsequent insertion of the SV40 gene.

Each of the elements mentioned above must be functionally linked. In this context, the term “functionally linked” means that these elements are linked to each other such that the elements each exert their functions to enhance the expression of the gene to be expressed.

Examples of the vector to which the expression cassette is inserted include plasmids, viral vectors such as adenovirus vectors, adeno-associated virus vectors, lentivirus vectors, retrovirus vectors, herpesvirus vectors, and Sendai virus vectors, and non-viral vectors such as biodegradable polymers. The vector harboring the expression cassette can be transferred to cells by a method known in the art such as infection or electroporation.

Alternatively, this transfer may be carried out using a transection reagent known in the art.

The vector having the insert of the expression cassette of the present invention can be transferred to cells to transfect the cells, thereby allowing the cells to express the gene of interest and produce the protein of interest. An eukaryotic cell or prokaryotic cell system can be used for the transfer of the expression cassette of the present invention and the production of the protein of interest. Examples of the eukaryotic cells include established mammalian cell systems, insect cell systems, and cells such as filamentous fungus cells and yeast cells. Examples of the prokaryotic cells include cells of bacteria such as E. coli, Bacillus subtilis, and Brevibacillus bacteria. Preferably, mammalian cells such as Hela cells, HEK293 cells, CHO cells, COS cells, BHK cells, or Vero cells are used. Particularly, use of the system mentioned above in human cells allows fusion proteins to be efficiently prepared in large amounts. The host cells thus transformed can be cultured in vitro or in vivo to produce the protein of interest. The culture of the host cells is carried out by a method known in the art. For example, a medium for culture known in the art such as DMEM, MEM, RPMI1640, or IMDM can be used as a culture solution. The expressed protein can be purified by a method known in the art from the culture solution (in the case of a secretory protein) or from cell extracts (in the case of a non-secretory protein). For the expression and production of each protein of interest, the cells may be cotransfected with a plurality of vectors comprising different genes of interest. In this way, a plurality of proteins can be produced at once.

In order to allow the host cells to extracellularly secrete each expressed fusion protein, a DNA encoding a signal peptide may be linked to the gene of the fusion protein. A DNA encoding the signal peptide of the cancer-specific antigen such as PSA, PAP, PSMA, MAGEA4, CD147, or CEA may be used as the DNA encoding a signal peptide. Preferably, a signal peptide-encoding DNA of REIC/Dkk-3 gene is used. Use of such a signal peptide allows mammalian cells (e.g., 293 cells) used as the host cells to extracellularly secrete a large amount of fusion proteins. The nucleotide sequence of the REIC/Dkk-3 gene is disclosed in, for example, WO2008/050898. The DNA encoding the cancer-specific antigen such as PSA, PAP, PSMA, MAGEA4, CD147, or CEA may be incorporated in advance upstream of the multicloning site in the expression cassette. The DNA encoding the cytokine can be inserted to the multicloning site in the expression system used to prepare each fusion protein of the cancer-specific antigen such as PSA, PAP, PSMA, MAGEA4, CD147, or CEA with the cytokine.

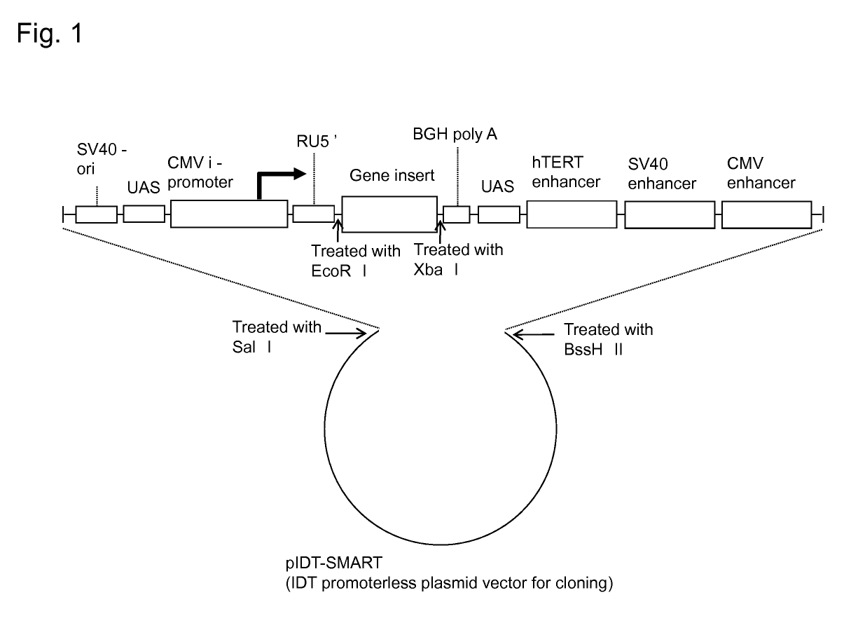

The exemplary structures of such an expression cassette are shown in FIGS. 1, 2, 3-1, and 3-2. The present invention also encompasses a DNA construct in which a DNA encoding any of 48 types of fusion proteins represented by PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, PAP-mGMCSF, PSMA-hIL2, PSMA-hIL4, PSMA-hIL7, PSMA-hGMCSF, PSMA-mIL4, PSMA-mGMCSF, MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF, CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4, CEA2-mGMCSF is inserted in a gene insert moiety in any of the expression cassettes shown in FIGS. 1, 2, 3-1, and 3-2. The present invention further encompasses a plasmid or a vector comprising the construct. The present invention further encompasses a pharmaceutical preparation which is a preparation for the treatment of a cancer that may be used in gene therapy, comprising the plasmid or the vector.

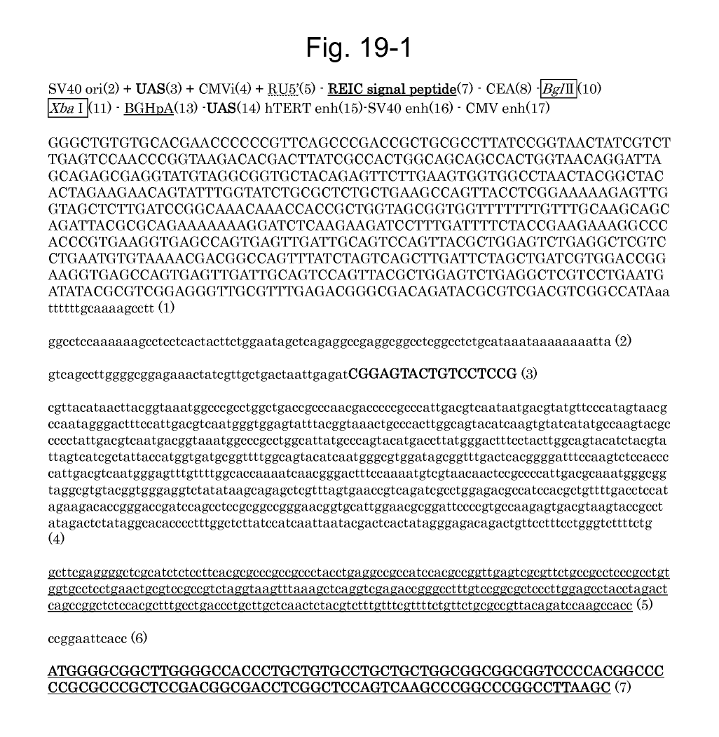

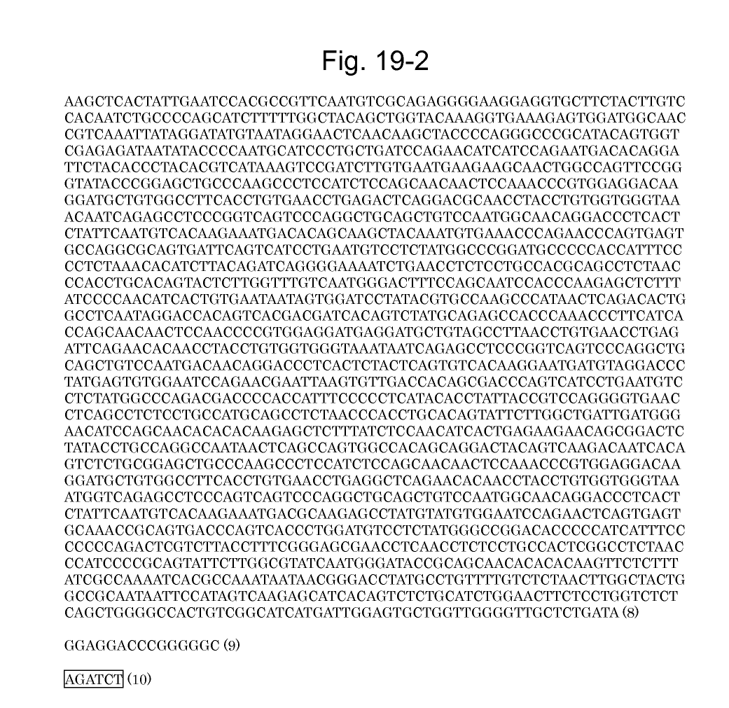

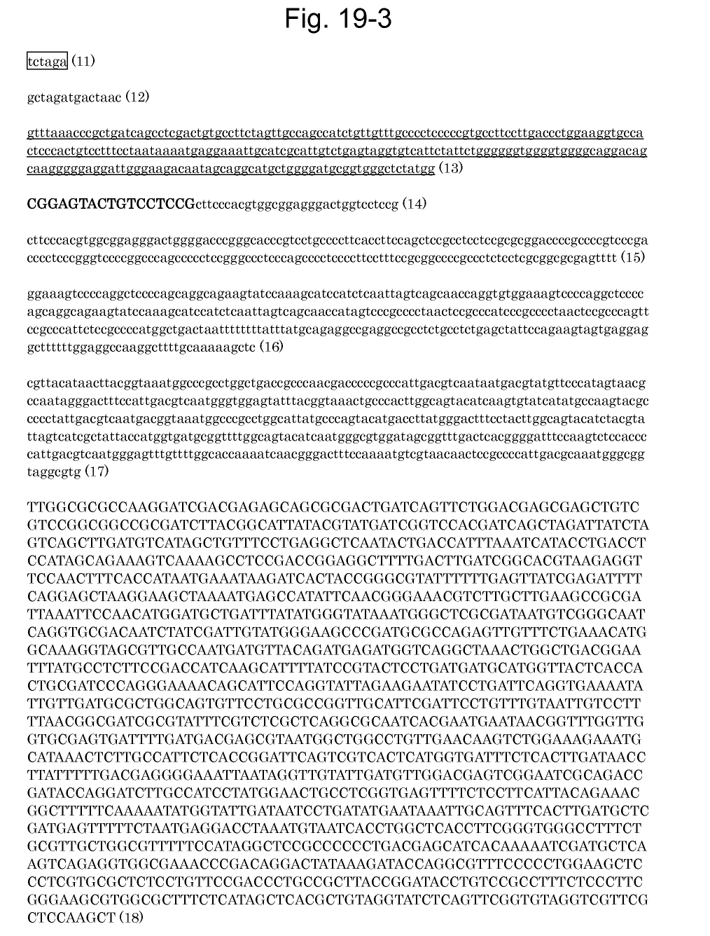

FIGS. 4-1 and 4-2 show the sequence of the expression cassette comprising the DNA encoding PSA (SEQ ID NO: 1). FIGS. 5-1 and 5-2 show the sequence of the expression cassette comprising the DNA encoding PAP (SEQ ID NO: 2). FIGS. 13-1, 13-2, and 13-3 show the sequence of the expression cassette comprising the DNA encoding PSMA (SEQ ID NO: 10). FIGS. 16-1, 16-2, and 16-3 show the sequence of the expression cassette comprising the DNA encoding MAGEA4 (SEQ ID NO: 11). FIGS. 17-1 and 17-2 show the sequence of the expression cassette comprising the DNA encoding CD147 (SEQ ID NO: 12). FIGS. 19-1, 19-2, and 19-3 show the sequence of the expression cassette comprising the DNA encoding CEA (SEQ ID NO: 13).

The effects of the thus-obtained fusion proteins of these cancer-specific antigens with each cytokine are based on: the uptake of the cancer-specific antigens by antigen-presenting precursor cells (monocytes, etc.) through receptors for the cytokine fused therewith; and the antitumor immunity-activating function of each cytokine itself

Hereinafter, the effects and application of the fusion proteins comprising PSA, PAP, PSMA, MAGEA4, CD147, or CEA as a cancer-specific antigen will be described in detail.

The fusion protein comprising PSA as a cancer-specific antigen is useful in the treatment of human prostate cancer expressing PSA and the prevention of recurrence thereof. The fusion protein of PSA and each cytokine is administered to a test subject where in vivo antigen-presenting cells such as dendritic cells can in turn present PSA to other immunocompetent cells to activate immunity against the antigen PSA and consequently activate immunity against PSA-expressing cancer cells, thereby shrinking prostate cancer tumor. The fusion protein of PSA and each cytokine, specifically, PSA-hGMCSF, acts on monocytes among human PBMCs to promote their differentiation into dendritic cells capable of presenting the antigen PSA. PSA-hIL4 acts on monocytes and lymphocytes among human PBMCs to promote the differentiation of the monocytes into dendritic cells capable of presenting the antigen PSA, while activating the lymphocytes having anticancer effects. PSA-hIL2 acts on lymphocytes and monocytes among human PBMCs to activate the lymphocytes having anticancer effects, while promoting the differentiation of the monocytes into dendritic cells capable of presenting the antigen PSA. PSA-hIL7 acts on lymphocytes and monocytes among human PBMCs to activate the lymphocytes having anticancer effects, while promoting the differentiation of the monocytes into dendritic cells capable of presenting the antigen PSA.

The fusion protein comprising PAP as a cancer-specific antigen is useful in the treatment of human prostate cancer expressing PAP and the prevention of recurrence thereof. The fusion protein of PAP and each cytokine is administered to a test subject where in vivo antigen-presenting cells such as dendritic cells can in turn present PAP to other immunocompetent cells to activate immunity against the antigen PAP and consequently activate immunity against PAP-expressing cancer cells, thereby shrinking prostate cancer tumor. The fusion protein of PAP and each cytokine, specifically, PAP-hGMCSF, acts on monocytes among human PBMCs to promote their differentiation into dendritic cells capable of presenting the antigen PAP. PAP-hIL4 acts on monocytes and lymphocytes among human PBMCs to promote the differentiation of the monocytes into dendritic cells capable of presenting the antigen PAP, while activating the lymphocytes having anticancer effects. PAP-hIL2 acts on lymphocytes and monocytes among human PBMCs to activate the lymphocytes having anticancer effects, while promoting the differentiation of the monocytes into dendritic cells capable of presenting the antigen PAP. PAP-hIL7 acts on lymphocytes and monocytes among human PBMCs to activate the lymphocytes having anticancer effects, while promoting the differentiation of the monocytes into dendritic cells capable of presenting the antigen PAP.

The fusion protein comprising PSMA as a cancer-specific antigen is useful in the treatment of human prostate cancer expressing PSMA and the prevention of recurrence thereof. The fusion protein of PSMA and each cytokine is administered to a test subject where in vivo antigen-presenting cells such as dendritic cells can in turn present PSMA to other immunocompetent cells to activate immunity against the antigen PSMA and consequently activate immunity against PSMA-expressing cancer cells, thereby shrinking prostate cancer tumor. The function effects of the fusion protein of PSMA and each cytokine are similar to those of the fusion protein of PSA or PAP.

The fusion protein comprising MAGEA4 as a cancer-specific antigen is useful in the treatment of a wide range of cancer types including melanoma, breast cancer, lung cancer, stomach cancer, bladder cancer, hepatocellular cancer, esophagus cancer, brain tumor, and blood cancer expressing MAGEA4 and the prevention of recurrence thereof. The fusion protein of MAGEA4 and each cytokine is administered to a test subject where in vivo antigen-presenting cells such as dendritic cells can in turn present MAGEA4 to other immunocompetent cells to activate immunity against the antigen MAGEA4 and consequently activate immunity against MAGEA4-expressing cancer cells, thereby shrinking prostate cancer tumor. The function effects of the fusion protein of MAGEA4 and each cytokine are similar to those of the fusion protein of PSA or PAP.

The fusion protein comprising CD147 as a cancer-specific antigen is useful in the treatment of a wide range of cancer types including bladder cancer, breast cancer, lung cancer, mouth cancer, esophagus cancer, skin cancer, malignant lymphoma, glioma, ovary cancer, melanoma, and hepatocellular cancer expressing CD147 and the prevention of recurrence thereof. The fusion protein of CD147 and each cytokine is administered to a test subject where in vivo antigen-presenting cells such as dendritic cells can in turn present CD147 to other immunocompetent cells to activate immunity against the antigen CD147 and consequently activate immunity against CD147-expressing cancer cells, thereby shrinking prostate cancer tumor. The function effects of the fusion protein of CD147 and each cytokine are similar to those of the fusion protein of PSA or PAP.

The fusion protein comprising CEA as a cancer-specific antigen is useful in the treatment of a wide range of cancer types including large intestine cancer, rectal cancer, thyroid gland cancer, esophagus cancer, stomach cancer, breast cancer, gallbladder cancer, bile duct cancer, lung cancer, pancreatic cancer, uterine cervix cancer, ovary cancer, bladder cancer, and medullary thyroid cancer expressing CEA and the prevention of recurrence thereof. The fusion protein of CEA and each cytokine is administered to a test subject where in vivo antigen-presenting cells such as dendritic cells can in turn present CEA to other immunocompetent cells to activate immunity against the antigen CEA and consequently activate immunity against CEA-expressing cancer cells, thereby shrinking prostate cancer tumor. The function effects of the fusion protein of CEA and each cytokine are similar to those of the fusion protein of PSA or PAP.

In the case of using mouse IL4 (mIL4) instead of hIL4, the resulting fusion proteins can exert effects similar to those of PSA-hIL4 or PAP-hIL4 even on a human test subject. In the case of using mouse GMCSF (mGMCSF) instead of hGMCSF, the resulting fusion proteins can exert effects similar to those of PSA-hGMCSF or PAP-hGMCSF even on a human test subject. The same holds true for use of the cancer-specific antigen PSMA, MAGEA4, CD147, or CEA.

These PSA- or PAP-cytokine fusion proteins can be used alone or in combination of two or more thereof as a therapeutic agent for prostate cancer by direct administration (through subcutaneous, intramuscular, or intravenous injection, etc.) to a prostate cancer patient. The combination may be a combination of fusion proteins of the same cytokine with different cancer-specific antigens or may be combination of fusion proteins of the same cancer-specific antigen with different cytokines. For example, 12 types of fusion proteins PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, and PAP-mGMCSF can be used in arbitrary combination of 2 types, 3 types, 4 types, 5 types, 6 types, 7 types, 8 types, 9 types, 10 types, 11 types, or 12 types in the treatment of prostate cancer. In addition to the PSA- or PAP-cytokine fusion proteins, PSMA-cytokine fusion protein(s) can be used as a therapeutic agent for prostate cancer. These PSMA-cytokine fusion proteins may each be used alone or in combination with the PSA- or PAP-cytokine fusion protein(s). For example, 18 types of fusion proteins, i.e., 6 types of fusion proteins PSMA-hIL2, PSMA-hIL4, PSMA-hIL7, PSMA-hGMCSF, PSMA-mIL4 and PSMA-mGMCSF plus the above-mentioned 12 types of PSA- or PAP-cytokine fusion proteins, can be used in arbitrary combination of 2 types, 3 types, 4 types, 5 types, 6 types, 7 types, 8 types, 9 types, 10 types, 11 types, 12 types, 13 types, 14 types, 15 types, 16 types, 17 types, or 18 types in the treatment of prostate cancer.

Furthermore, the fusion proteins of MAGEA4, CD147, or CEA with various cytokines may be used in combination. For example, 30 types of fusion proteins MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF, CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4, CEA2-mGMCSF can be used in arbitrary combination of 2 types, 3 types, 4 types, 5 types, 6 types, 7 types, 8 types, 9 types, 10 types, 11 types, 12 types, 13 types, 14 types, 15 types, 16 types, 17 types, 18 types, 19 types, 20 types, 21 types, 22 types, 23 types, 24 types, 25 types, 26 types, 27 types, 28 types, 29 types, or 30 types in the treatment of various cancers.

These fusion protein preparations can also be used in the treatment of a cancer such as prostate cancer which involves adding the fusion protein preparations alone or in combination of two or more thereof into a culture solution containing blood-derived cells such as mononuclear cells obtained from human peripheral blood, bone marrow fluid, umbilical cord blood, or the like; culturing these cells to simultaneously activate ex vivo monocytes, lymphocytes, etc.; and then administering these activated antitumor immunocytes into the body of a patient. In this method, the blood-derived cells such as PBMCs obtained from human peripheral blood or the like can be cultured with the fusion proteins each comprising the cancer-specific antigen such as PSA, PAP, PSMA, MAGEA4, CD147, CEA and each cytokine to thereby prepare immunocompetent cells including antigen-presenting cells such as dendritic cells having antitumor activity and activated lymphocytes such as cytotoxic T lymphocytes, helper T lymphocytes, or B lymphocytes. The present invention also encompasses a method for preparing in vitro a dendritic cell having antitumor activity based on strong antigen-presenting ability, using these fusion proteins. The dendritic cells thus obtained present the cancer-specific antigen such as PSA, PAP, PSMA, MAGEA4, CD147, or CEA and exhibit antitumor immunity activity when administered to an organism. In the present invention, the dendritic cells having antitumor immunity activity are also referred to as antitumor immunity-activated dendritic cells. The fusion proteins can also be used as an antitumor immunity activator for dendritic cells. In this case, the own blood-derived cells of a test subject having a cancer to be prevented or treated can be used. These cells can be treated and then brought back to the test subject. Instead of the blood-derived cells, cells capable of differentiating into immunocompetent cells (blood-derived cell), i.e., stem cells, may be used. Examples of such cells include induced pluripotent stem (iPS) cells, embryonic stem cells (ES cells), blood stem cells including hematopoietic stem cells in the bone marrow, mesenchymal stem cells, various tissue-specific stem cells, and other pluripotent stem cells. In the case of using these stem cells, the stem cells can be treated with the fusion proteins of the present invention ex vivo and then used in immunotherapy.

The blood-derived cells such as mononuclear cells obtained from human peripheral blood, bone marrow fluid, umbilical cord blood, or the like as well as cells capable of differentiating into the blood-derived cells can differentiate into immunocompetent cells. In the present invention, these cells are therefore referred to as cells capable of differentiating into immunocompetent cells.

As described above, the present invention encompasses antitumor immunotherapy which involves adding the fusion proteins of the present invention to blood-derived cells collected from a test subject through apheresis, culturing these cells, and bringing the resultant cells back to the body of the test subject.

In the present invention, each antigen protein or cells expressing the antigen protein are used as a therapeutic target. For a mechanism underlying the activation of immunity against each target, it is important that each fusion protein (population) should be able to induce both of cytotoxic T lymphocytes (CD8-positive) and B lymphocytes (CD19-positive). Specifically, this can be expected to activate both functions, i.e., cellular immunity [effects brought about by cytotoxic T lymphocytes (CD8-positive)] and humoral immunity [effects based on the antibody functions such as antibody-dependent cell-mediated cytotoxicity (ADCC) of B lymphocytes (CD19-positive)] against each cancer antigen (provided that CD147 serves not only as a cancer antigen but as an antigen causative of or related to the pathological conditions of a wide range of diseases). The induction of dendritic cells (CD86-positive) and helper T lymphocytes (CD4-positive) by each fusion protein (population) contributes to the activation of both of the cellular immunity and the humoral immunity.

In the case of using PSA, PAP, or PSMA as a cancer-specific antigen, the cancer to be prevented or treated is, as described above, prostate cancer. Alternatively, the cancer-specific antigen such as MAGEA4, CD147, or CEA can be selected to thereby target brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, mesothelioma, etc.

CD147 is a member of the immunoglobulin superfamily that is expressed on the cells of various tissues. This protein is involved in fetal development, retinal functions, T cell maturation, etc. CD147 is expressed in tumors, the endometrium, the placenta, the skin, and regions undergoing angiogenesis and stimulates matrix metalloproteinase (MMP) and VEGF production. CD147 is induced by the differentiation of monocytes and expressed in human atheroma. CD147 is also involved in the promotion of infiltration or metastasis of different tumor types via the induction of MMP and urokinase-type plasminogen activator systems by peritumoral stromal cells. In addition, CD147 is also involved in angiogenesis, anoikis resistance, lactate release, multidrug resistance, and cancer cell growth. The overexpression or excessive functions of CD147 are also related to inflammatory reaction, pulmonary fibrosis, rheumatoid arthritis, lupus erythematosus, heart failure, Alzheimer's disease, and other pathological processes such as the infectious cycles of human immunodeficiency virus and coronavirus in lymphocytes. Thus, CD147 is not only specifically expressed on cancer cells but involved in various diseases other than cancers. Specifically, CD147 is associated with malignant diseases caused by, for example, the tumor cell-mediated MMP stimulation, VEGF release, and angiogenesis promotion of neuroblasts. Use of each CD147 fusion protein of the present invention can inhibit the biological activity of CD147, thereby treating or preventing a disease that is developed by the involvement of CD147 activity. Thus, the fusion protein comprising CD147 can target cell populations responsible for a wide range of diseases other than cancers to treat the diseases. It has been reported as to various diseases that the presence, expression, increased expression, activation, or the like of CD147 is involved in the onset, preservation, or aggravation of the pathological conditions of these diseases (e.g., WO2010/036460). Examples of the diseases involving CD147 include cancers as well as thrombogenic diseases (myocardial infarction, cerebral infarction, etc.), COPD, MS, ALS, inflammatory diseases, malaria, liver cirrhosis, diseases that are desirably treated by the inhibition of Treg, systemic sclerosis (SS), rheumatoid arthritis, and Alzheimer's disease.

The CD147 fusion proteins of the present invention, specifically, CD147-hIL2, CD147-hIL4, CD147-hIL7, CD147-hGMCSF, CD147-mIL4, and CD147-mGMCSF, can be used alone or in combination in the treatment or prevention of diseases or conditions listed below.

The CD147 fusion protein preparations can also be used in the treatment of a disease involving CD147 which involves adding the fusion protein preparations alone or in combination of two or more thereof into a culture solution containing blood-derived cells such as mononuclear cells obtained from human peripheral blood, bone marrow fluid, umbilical cord blood, or the like; culturing these cells to simultaneously activate ex vivo monocytes, lymphocytes, etc.; and then administering these activated cells into the body of a patient. In this method, the blood-derived cells such as PBMCs obtained from human peripheral blood or the like can be cultured with the fusion proteins each comprising CD147 and each cytokine to thereby prepare immunocompetent cells including antigen-presenting cells such as dendritic cells targeting CD147-expressing cells and activated lymphocytes such as cytotoxic T lymphocytes, helper T lymphocytes, or B lymphocytes. The present invention also encompasses a method for preparing in vitro a dendritic cell targeting CD147-expressing cells based on strong antigen-presenting ability, using the CD147 fusion proteins. The dendritic cells thus obtained present CD147 and exhibit the activity of attacking CD147-expressing cells when administered to an organism. In this case, the own blood-derived cells of a test subject having a disease involving CD147 to be prevented or treated can be used. These cells can be treated and then brought back to the test subject. Instead of the blood-derived cells, cells capable of differentiating into immunocompetent cells (blood-derived cell), i.e., stem cells, may be used. Examples of such cells include induced pluripotent stem (iPS) cells, embryonic stem cells (ES cells), blood stem cells including hematopoietic stem cells in the bone marrow, mesenchymal stem cells, various tissue-specific stem cells, and other pluripotent stem cells. In the case of using these stem cells, the stem cells can be treated with the CD147 fusion proteins of the present invention ex vivo and then used in immunotherapy.

The blood-derived cells such as mononuclear cells obtained from human peripheral blood, bone marrow fluid, umbilical cord blood, or the like as well as cells capable of differentiating into the blood-derived cells can differentiate into immunocompetent cells. In the present invention, these cells are therefore referred to as cells capable of differentiating into immunocompetent cells.

As described above, the present invention encompasses therapy which involves adding the CD147 fusion proteins of the present invention to blood-derived cells collected from a test subject through apheresis, culturing these cells, and bringing the resultant cells back to the body of the test subject.

These CD147 fusion proteins can be used in the treatment or prevention of a disease or a condition involving CD147. Examples of the condition involving CD147 include diseases or conditions mediated by cell migration and tissue remodeling in tissue regrowth, neoplastic diseases, metastatic diseases, and fibrotic states. These diseases or conditions include, for example, malignant and nervous system diseases. The CD147-related conditions include inflammatory or autoimmune diseases, cardiovascular diseases, and infections. The CD147 fusion proteins of the present invention are also useful in the treatment of a disease involving vascular formation and are useful in the treatment of, for example, eye diseases, neoplastic diseases, tissue reformation or growth of certain types of cells such as restenosis, particularly, epithelial and squamous cell cancers. The CD147 fusion proteins of the present invention may be further used in the treatment of atherosclerosis, restenosis, cancer metastasis, rheumatoid arthritis, diabetic retinopathy, or macular degeneration. The CD147 fusion proteins of the present invention may be further used in the prevention or treatment of bone resorption or bone degradation resulting from PTHrP overexpression found in osteoporosis or some tumors. In addition, the CD147 fusion proteins of the present invention may also be used in the prevention or treatment of idiopathic pulmonary fibrosis, diabetic nephropathy, hepatitis, and fibrosis such as liver cirrhosis.

The CD147 fusion proteins can also be used in the treatment of the following diseases.

Lung Diseases

Pneumonia; lung abscess; occupational lung disease caused by dust, gas, or an agent in the form of droplet; pulmonary hypersensitivity diseases including asthma, bronchiolitis fibrosa obliterans, respiratory failure, hypersensitivity pneumonia (extrinsic allergic alveolitis), allergic bronchopulmonary aspergillosis, and drug reaction; adult respiratory distress syndrome (ARDS), Goodpasture's syndrome, chronic obstructive airway disease (COPD), idiopathic interstitial lung diseases (e.g., idiopathic pulmonary fibrosis and sarcoidosis, desquamative interstitial pneumonia, acute interstitial pneumonia, respiratory bronchiolitis-related interstitial lung diseases, idiopathic bronchiolitis obliterans organizing pneumonia, lymphocytic interstitial pneumonia, Langerhans cell granulomatosis, and idiopathic pulmonary hemosiderosis); and acute bronchitis, pulmonary alveolar proteinosis, bronchiectasis, pleural disease, atelectatic lung, cystic fibrosis, lung tumor, and pulmonary embolism.

Malignant Diseases

Leukemia, acute leukemia, acute lymphoblastic leukemia (ALL), B cell, T cell, or FAB ALL, acute myeloid leukemia (AML), chronic myeloid leukemia (CML), chronic lymphocytic leukemia (CLL), hairy cell leukemia, myelodysplastic syndrome (MDS), lymphoma, Hodgkin's disease, malignant lymphoma, non-Hodgkin's lymphoma, Burkitt's lymphoma, multiple myeloma, solid tumors such as primary diseases or metastatic diseases, Kaposi's sarcoma, colorectal cancer, pancreatic cancer, renal cell cancer, lung cancer including mesothelioma, breast cancer, nasopharynx cancer, malignant histiocytosis, malignant paraneoplastic syndrome/hypercalcemia, adenocarcinoma, squamous cell cancer, sarcoma, malignant melanoma, particularly, metastatic melanoma, angioma, metastatic diseases, cancer-related bone resorption, and cancer-related bone pain.

Immunity-Related Diseases

Rheumatoid arthritis, juvenile rheumatoid arthritis, systemic juvenile rheumatoid arthritis, psoriatic arthritis, ankylosing spondylitis, gastric ulcer, seronegative arthropathy, osteoarthritis, inflammatory bowel disease, ulcerative colitis, systemic lupus erythematosus, antiphospholipid syndrome, iridocyclitis/uveitis/optic neuritis, idiopathic pulmonary fibrosis, systemic vasculitis/Wegener's granulomatosis, sarcoidosis, orchitis/vasectomy reversal procedure, allergic or atopic diseases, asthma, allergic rhinitis, eczema, allergic contact dermatitis, allergic conjunctivitis, hypersensitivity pneumonia, graft rejection, organ transplant rejection, graft-versus-host disease, systemic inflammation response syndrome, sepsis syndrome, Gram-positive sepsis, Gram-negative sepsis, culture-negative sepsis, fungal sepsis, neutropenic fever, urosepsis, meningococcemia, trauma/bleeding, burn, exposure to ionizing radiation, acute pancreatitis, adult respiratory distress syndrome, rheumatoid arthritis, alcoholic hepatitis, chronic inflammatory diseases, sarcoidosis, Crohn's pathology, sickle cell anemia, diabetes mellitus, nephrosis, atopic diseases, hypersensitivity, allergic rhinitis, hay fever, perennial rhinitis, conjunctivitis, endometriosis, asthma, urticaria, systemic anaphylaxis, dermatitis, pernicious anemia, hemolytic disease, thrombocytopenia, rejection of any organ or tissue graft, kidney transplant rejection, heart transplant rejection, liver transplant rejection, pancreas transplant rejection, lung transplant rejection, bone marrow transplant (BMT) rejection, skin transplant rejection, cartilage transplant rejection, bone graft rejection, small intestine transplant rejection, fetal thymus transplant rejection, parathyroid gland transplant rejection, rejection of any organ or tissue xenograft, rejection of any organ or tissue allograft, anti-receptor hypersensitivity reaction, Graves' disease, Raynaud's disease, type B insulin-resistant diabetes mellitus, asthma, myasthenia gravis, antibody-mediated cytotoxicity, type III hypersensitivity reaction, systemic lupus erythematosus, POEMS syndromes (polyneuropathy, organomegaly, endocrine disease, monoclonal gammopathy, and skin change syndrome), antiphospholipid syndrome, pemphigus, pachyderma, mixed connective-tissue disease, idiopathic Addison's disease, genuine diabetes, chronic active hepatitis, primary biliary cirrhosis, leukoderma, vasculitis, post-MI cardiotomy syndrome, type IV hypersensitivity, contact dermatitis, hypersensitivity pneumonia, allograft rejection, granuloma caused by intracellular microorganisms, drug sensitivity, metabolism/idiopathy, Wilson's disease, hemachromatosis, α-1-antitrypsin deficiency, diabetic retinopathy, Hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatological manifestation, psoriasis, alopecia, nephrotic syndrome, nephritis, glomerulonephritis, acute renal failure, hemodialysis, uremia, toxicity, preeclampsia, OKT3 therapy, anti-CD3 therapy, cytokine therapy, chemotherapy, radiotherapy (e.g., asthma, anemia, and cachexia), and chronic salicylate intoxication.

Cardiovascular Diseases

Cardiac stun syndrome, myocardial infarction, congestive heart failure, apoplexy, ischemic episodes, bleeding, arteriosclerosis, atherosclerosis, restenosis, diabetic ateriosclerotic disease, hypertension, arterial hypertension, renovascular hypertension, syncope, shock, cardiovascular syphilis, heart failure, cor pulmonale, primary pulmonary hypertension, arrhythmia, atrial ectopic beat, atrial flutter, atrial fibrillation (persistent or paroxysmal), postperfusion syndrome, inflammatory response to cardiopulmonary bypass, chaotic or multifocal atrial tachycardia, regular narrow QRS tachycardia, specific arrythmias, ventricular fibrillation, His bundle arrythmias, atrioventricular block, bundle branch block, myocardial ischemic disease, coronary disease, angina pectoris, myocardial infarction, cardiomyopathy, dilated congestive cardiomyopathy, restrictive cardiomyopathy, valvular heart disease, endocarditis, pericardial disease, heart tumor, aortal aneurysm or peripheral arterial aneurysm, aortotomy, aortitis, occlusion of abdominal aorta and bifurcation thereof, peripheral vascular disease, occlusive arterial disease, peripheral atherosclerotic disease, thromboangiitis obliterans, functional peripheral arterial disease, Raynaud's phenomenon and disease, acrocyanosis, erythromelalgia, venous disease, venous thrombosis, varicose vein, arteriovenous fistula, lymphedema, lipedema, unstable angina, reperfusion injury, post pump syndrome, and ischemic reperfusion injury.

Nervous System Diseases

Demyelinating diseases such as neurodegenerative disease, multiple sclerosis, migraine, AIDS-dementia complex, multiple sclerosis, and acute transverse myelitis; extrapyramidal and cerebellar diseases such as lesions in the corticospinal system; basal ganglion diseases or cerebellar diseases; hyperactivity disorders such as Huntington's chorea and senile chorea; drug-induced movement disorders including ones induced by drugs blocking CNS dopamine receptors; hypokinetic disorders such as Parkinson's disease; progressive supranuclear palsy; organic lesions in the cerebellum; spinocerebellar degenerations such as spinal ataxia, Friedreich ataxia, spinocerebellar degenerations, and multiple systems degenerations (Mencel, Dejerine-Thomas, Shi-Drager, and Machado-Joseph); systemic diseases (Refsum's disease, abeta-lipoproteinemia, ataxia, telangiectasis, and mitochondrial multi. system disorder); demyelinating core disorders such as multiple sclerosis and acute transverse myelitis; motor unit disorders such as neural muscular atrophy (anterior horn cell degenerations such as amyotrophic lateral sclerosis, infantile spinal muscular atrophy, and juvenile spinal muscular atrophy); Alzheimer's disease; Down's syndrome in middle age; diffuse Lewy body disease; senile dementia with Lewy bodies; Wernicke-Korsakoff syndrome; chronic alcoholism; Creutzfeldt-Jakob disease; subacute sclerosing panencephalitis and Hallerrorden-Spatz disease; and dementia pugilistica.

Other Diseases

Hepatic fibrosis (alcoholic cirrhosis, viral cirrhosis, autoimmune hepatitis, etc.); pulmonary fibrosis (pachyderma, idiopathic pulmonary fibrosis, etc.); renal fibrosis (including, but not limited to, pachyderma, diabetic nephritis, glomerulonephritis, and lupus nephritis); skin fibrosis (pachyderma, hyperplastic and keloidal scars, burn, etc.); bone marrow fibrosis; neurofibromatosis; fibroma; intestinal fibrosis; and various fibrotic diseases such as fibrous adhesions resulting from surgical operation.

Acute or chronic bacterial infections, the processes of acute and chronic parasitism or injection including bacterial, viral, and fungal infections, HIV injection/HIV neuropathy, meningitis, hepatitis (A, B, or C, etc.), septic arthritis, peritonitis, pneumonia, epiglottitis, E. coli, hemolytic uremic syndrome, malaria, dengue hemorrhagic fever, leishmaniasis, Hansen's disease, toxic shock syndrome, streptococcal myositis, gas gangrene, Mycobacterium tuberculosis, Mycobacterium avium, Pneumocystis carinii pneumonia, pelvic inflammatory disease, orchitis/epididymitis, legionella, Lyme disease, type A influenza, Epstein-Barr virus, virus-associated hemophagocytic syndrome, and viral encephalitis/aseptic meningitis.

When the fusion proteins of the present invention are used as a pharmaceutical composition for the prevention or treatment of a cancer to be administered to a test subject, the pharmaceutical composition may contain a pharmacologically acceptable carrier, diluent, or excipient, in addition to the fusion proteins. For example, lactose or magnesium stearate is used as a carrier or an excipient for tablets. Saline, an isotonic solution containing glucose or an additional adjuvant, or the like is used as an aqueous solution for injection and may be used in combination with an appropriate solubilizing agent, for example, an alcohol, a polyalcohol (propylene glycol), or a nonionic surfactant. Sesame oil, soybean oil, or the like is used as an oily solution and may be used in combination with a solubilizing agent such as benzyl benzoate or benzyl alcohol.

The pharmaceutical composition can be administered in various forms. Examples thereof include orally administrable formulations such as tablets, capsules, granules, powders, and syrups, and parenterally administrable formulations such as injections, drops, suppositories, sprays, eye drops, transnasal agents, and patches. The pharmaceutical composition may be locally administered and can exert its effects by administration, for example, through injection to a cancer site. Preferably, the pharmaceutical composition is locally injected once or more times directly to a cancer lesion such that the active ingredients are spread throughout the cancer lesion.

The dose thereof differs depending on the symptoms, age, body weight, etc. of a recipient and can be 0.001 mg to 100 mg per dose which is administered every few days, few weeks, or few months through intravenous injection, intraperitoneal injection, subcutaneous injection, intramuscular injection, or the like.

When the fusion proteins of the present invention are used in ex vivo treatment, for example, PBMCs can be used at a concentration of 10⁴ to 10⁷ cells/ml and cultured with the fusion proteins added at a concentration of 1 to 50 μg/ml.

DNAs encoding the fusion proteins of the present invention can be used in gene therapy. For this purpose, the DNAs encoding the fusion proteins of the present invention can be inserted to appropriate vectors, which are then administered to an organism for in vivo expression of the fusion proteins. For example, any of DNAs encoding 48 types of fusion proteins represented by PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, PAP-mGMCSF, PSMA-hIL2, PSMA-hIL4, PSMA-hIL7, PSMA-hGMCSF, PSMA-mIL4, PSMA-mGMCSF, MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF, CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4, CEA2-mGMCSF is inserted to a gene insert moiety in the expression cassette of FIG. 1, 2, 3-1, or 3-2 to prepare a DNA construct. This DNA construct can be incorporated into a plasmid or a vector, which is then administered to an organism.

Examples of the plasmid or the vector to which the DNA construct is inserted include plasmids, viral vectors such as adenovirus vectors, adeno-associated virus vectors, lentivirus vectors, retrovirus vectors, herpesvirus vectors, and Sendai virus vectors, and non-viral vectors such as biodegradable polymers. The vector harboring the DNA construct can be transferred to cells by infection or the like. This transfer may be carried out using a transection reagent known in the art.

The plasmids or the vectors harboring the fusion protein-encoding DNAs can be administered by a method that may be used in the field of gene therapy, for example, intravascular administration (intravenous or intraarterial administration), oral administration, intraperitoneal administration, intratracheal administration, intrabronchial administration, subcutaneous administration, percutaneous administration, or the like.

The plasmids or the vectors harboring the fusion protein-encoding DNAs can be administered in a therapeutically effective amount. The therapeutically effective amount can be easily determined by those skilled in the field of gene therapy. The dose can be appropriately changed according to the pathological severity, sex, age, body weight, habit, etc. of a test subject. For example, adenovirus vectors or adeno-associated virus vectors harboring the fusion protein-encoding DNAs can be administered in an amount of 0.5×10¹¹ to 2.0×10¹² viral genomes/kg body weight, preferably 1.0×10¹¹ to 1.0×10¹² viral genomes/kg body weight, more preferably 1.0×10¹¹ to 5.0×10¹¹ viral genomes/kg body weight. The viral genomes represent the number of molecules of adenovirus or adeno-associated virus genomes (the number of virions) and are also referred to as particles. A carrier, a diluent, or an excipient usually used in the pharmaceutical field is contained therein. For example, lactose or magnesium stearate is used as a carrier or an excipient for tablets. Saline, an isotonic solution containing glucose or an additional adjuvant, or the like is used as an aqueous solution for injection and may be used in combination with an appropriate solubilizing agent, for example, an alcohol, a polyalcohol (propylene glycol), or a nonionic surfactant. Sesame oil, soybean oil, or the like is used as an oily solution and may be used in combination with a solubilizing agent such as benzyl benzoate or benzyl alcohol.

The gene therapy using such plasmids or vectors harboring the DNAs encoding the fusion proteins of the present invention can be carried out according to the description of, for example, International Publication No. WO2011/062298.

The present invention further encompasses a non-human animal model of a human cancer having a transplant of a human cancer cell highly expressing a cancer-specific antigen. The non-human animal includes mice, rats, rabbits, guinea pigs, dogs, cats, monkeys, and the like and is preferably a rodent such as a mouse or a rat. The non-human animal model of a human cancer can be prepared by: transforming a human cancer cell line with a gene encoding the cancer-specific antigen and transplanting the transformed cancer cell line to a non-human animal. For this transformation of the human cancer cell line, a DNA encoding the cancer-specific antigen is inserted to the expression cassette mentioned above that comprises (i) a DNA construct comprising a first promoter, a gene to be expressed, and a poly-A addition sequence linked in this order and (ii) an enhancer or an enhancer with UAS linked upstream thereof, in the order of (i) and (ii), and has a structure where the enhancer or the enhancer with UAS linked upstream thereof is linked immediately downstream of the poly-A addition sequence. The cancer cell line can be transformed with a plasmid harboring the expression cassette. A drug resistance gene such as a neomycin resistance gene can be incorporated into this expression cassette to thereby select a transformed cell line. The cancer-specific antigen used can be a cancer-specific antigen specific for the cancer type of the cancer cell line. For example, a prostate cancer cell line such as an RM-9 cell line can be transformed with a plasmid comprising a DNA encoding PSA or PAP. Such a transformed cell line is referred to as PSA-RM9 cells or PAP-RM9 cells. The obtained transformed cell line is transplanted to a non-human animal to thereby form the cancer and express the cancer-specific antigen. The non-human animal model of a human cancer thus obtained can exhibit a pathological condition similar to that of the human cancer. This non-human animal model of a human cancer can be used in, for example, the screening or evaluation of a therapeutic drug for the cancer.

### EXAMPLES

Hereinafter, the present invention will be specifically described with reference to Examples. However, the present invention is not intended to be limited by these Examples.

### Example 1 Production of PSA- or PAP-Containing Fusion Proteins

Fusion proteins of PSA (prostate-specific antigen) or PAP (prostatic acid phosphatase) with human IL2 (hIL2), human IL4 (hIL4), human IL7 (hIL7), human GMCSF (hGMCSF), mouse IL4 (mIL4), or mouse GMCSF (mGMCSF) were produced.

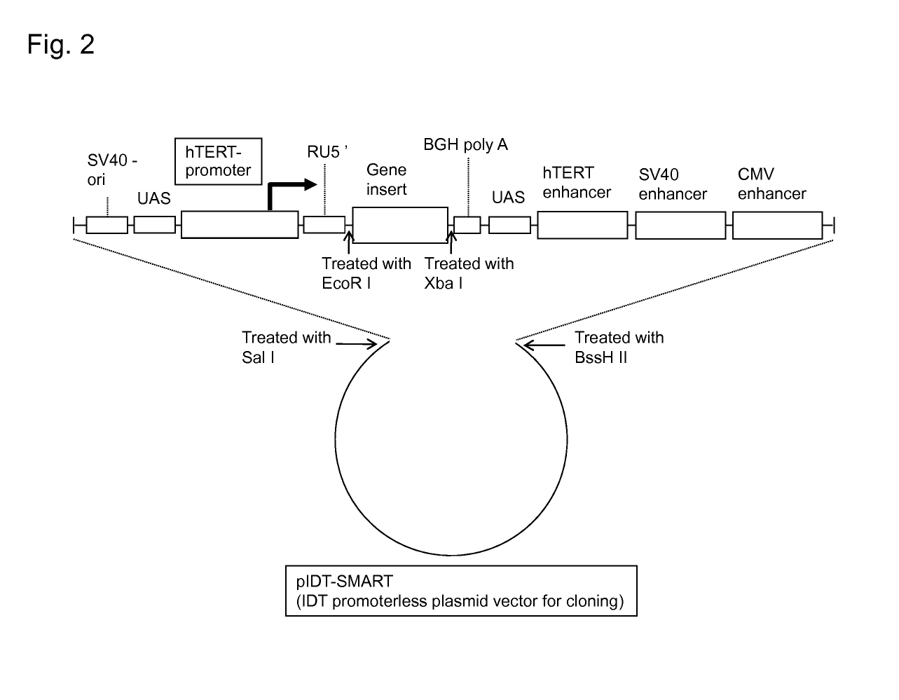

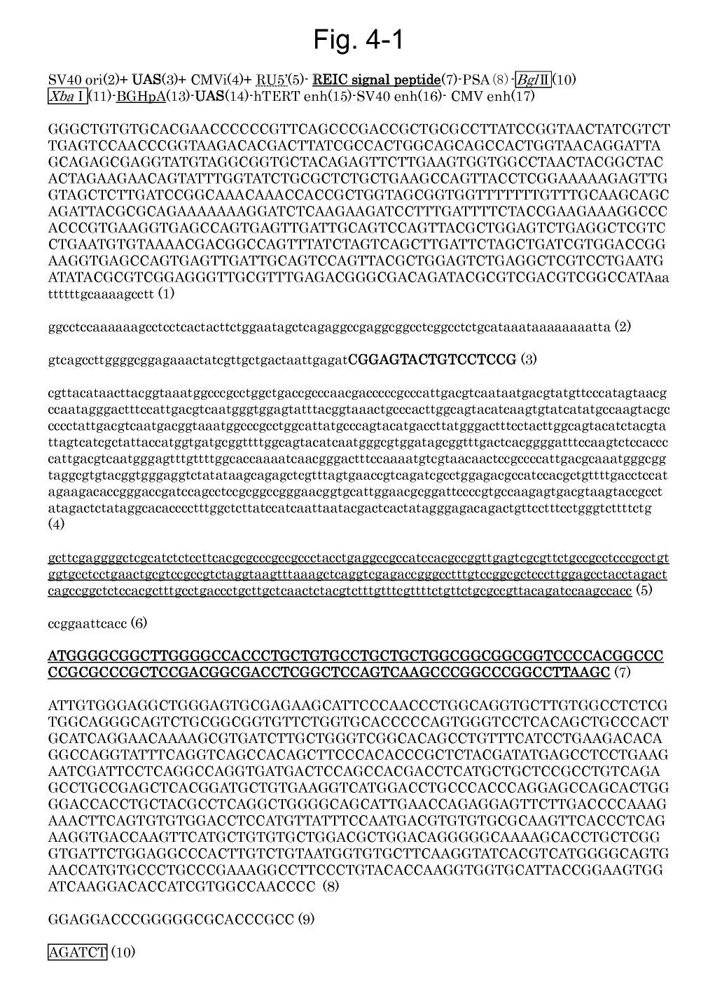

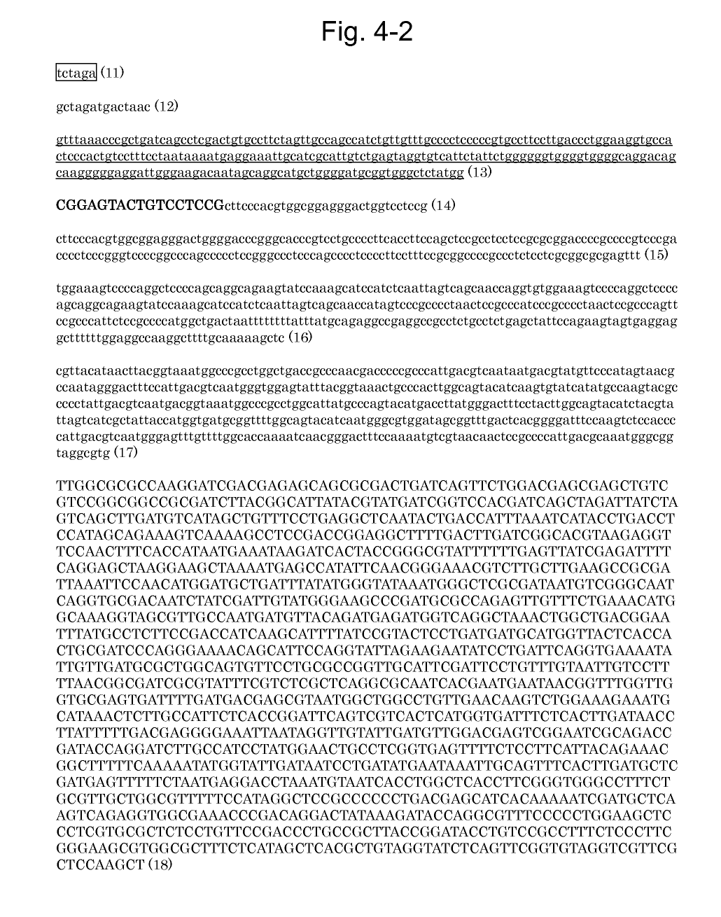

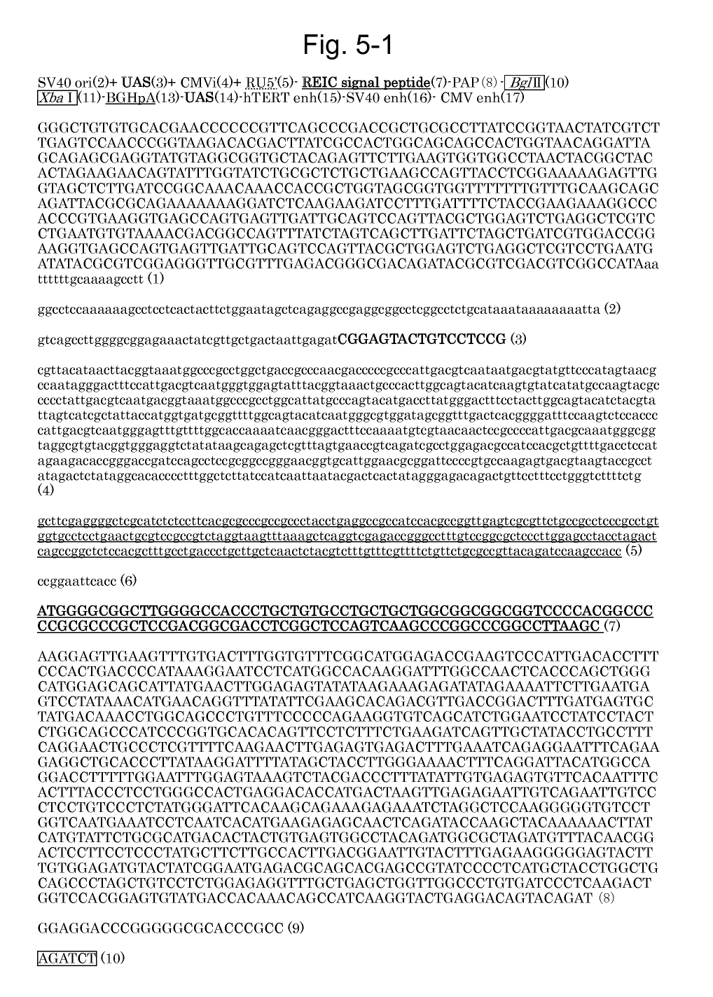

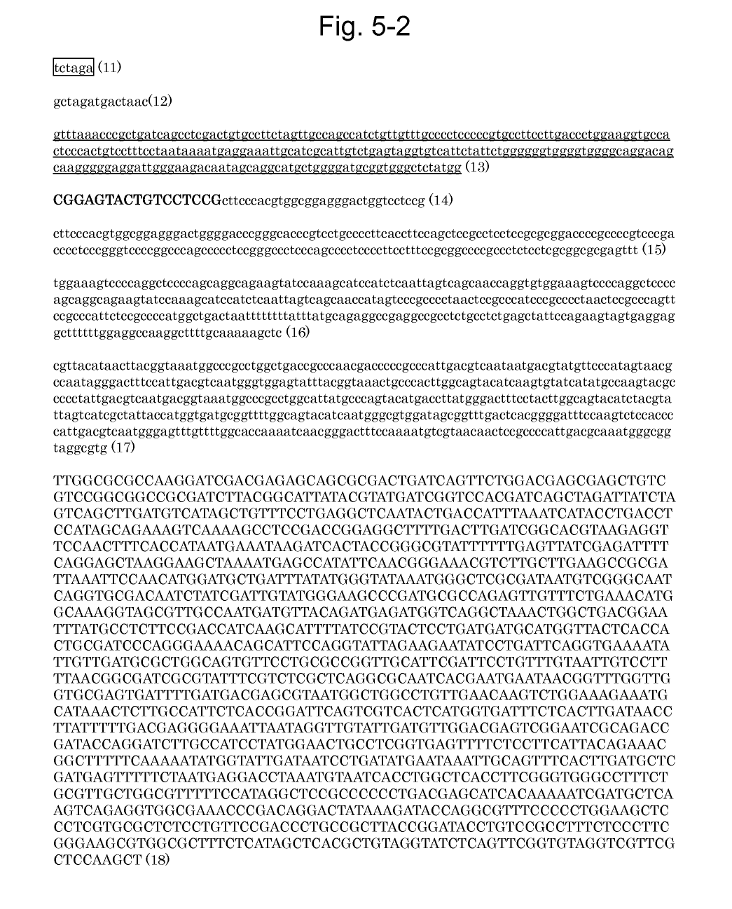

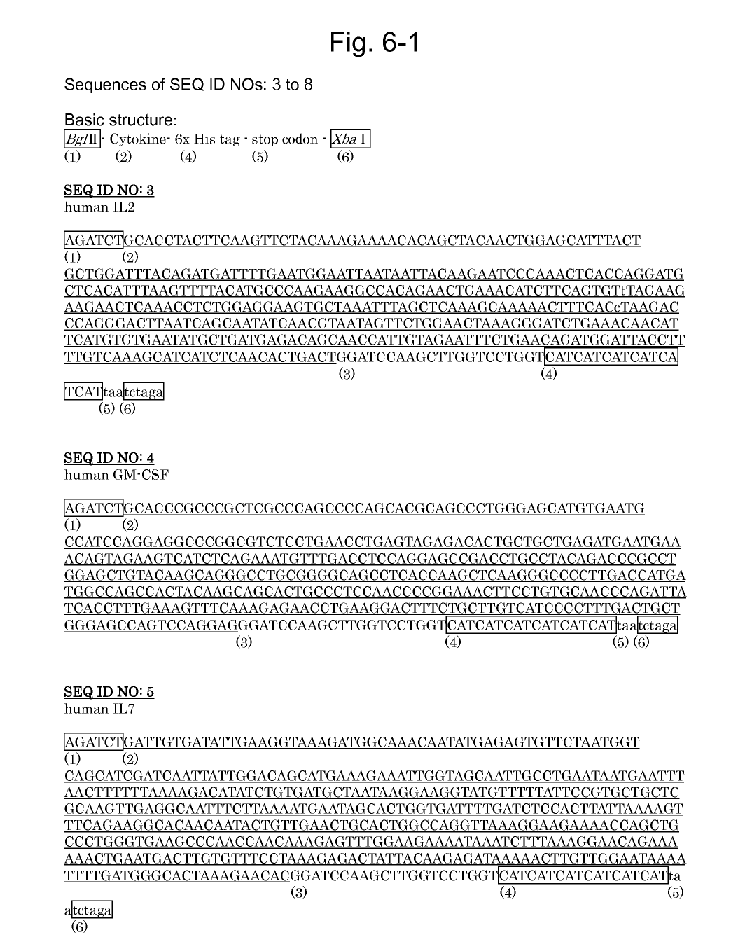

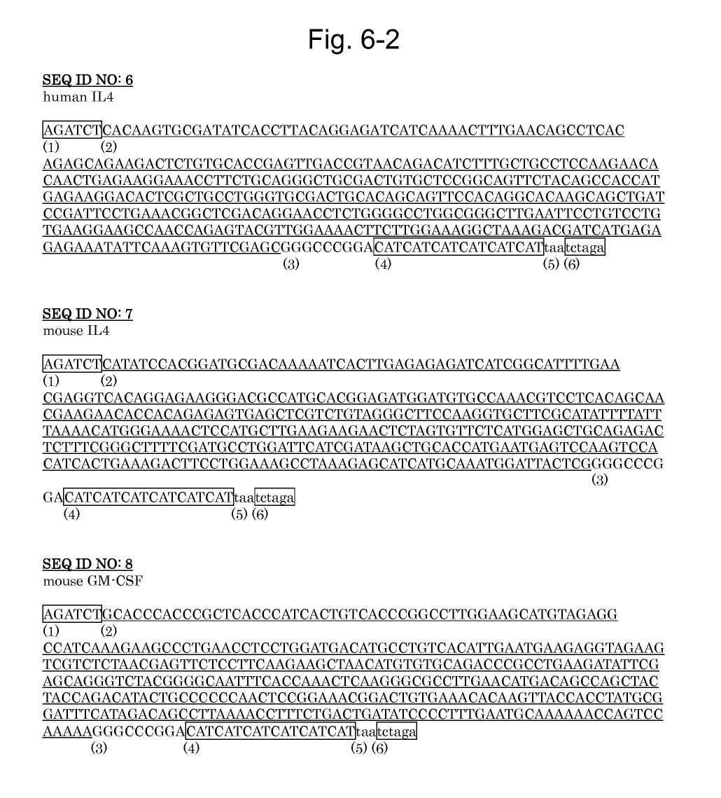

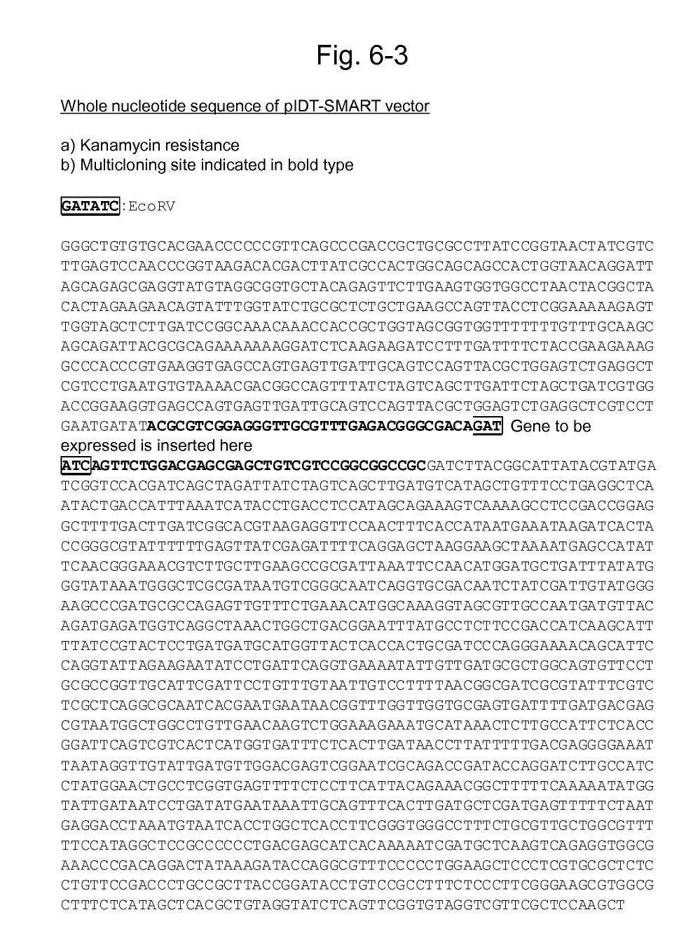

In this Example, the expression cassettes shown in FIGS. 4-1 and 4-2 and FIGS. 5-1 and 5-2 (their sequences are shown in SEQ ID NOs: 1 and 2, respectively) were used. The sequence shown in FIG. 4-2 is a sequel to the sequence shown in FIG. 4-1. A DNA encoding each cytokine is inserted to between the sequence shown in FIG. 4-1 and the sequence shown in FIG. 4-2 by use of restriction enzyme sites. Likewise, the sequence shown in FIG. 5-2 is a sequel to the sequence shown in FIG. 5-1. A DNA encoding each cytokine is inserted to between the sequence shown in FIG. 5-1 and the sequence shown in FIG. 5-2 by use of restriction enzyme sites. The sequences shown in FIGS. 4-1 and 4-2 or FIGS. 5-1 and 5-2 are consecutive sequences, which are however indicated on an element bases in order to represent what each element is. The sequence of each element and this element in the upper structure diagram of FIG. 4-1 or 5-1 were numbered to represent what the element is in the sequence of the expression cassette. These expression cassettes were prepared on the basis of an expression cassette having the structure shown in FIG. 1 and each have the structure shown in the upper area of FIG. 4-1 or FIG. 5-1. In the diagram showing the structure in the upper area of FIG. 4-1 or FIG. 5-1, SV40 ori (2) represents an SV40 gene-binding region. UAS (3) represents a GAL4 gene-binding region. CMVi (4) represents a CMVi promoter. RU5′ (5) represents HTLV-derived LTR. REIC signal peptide (7) represents a signal peptide-encoding DNA of a REIC/Dkk-3 gene sequence. PSA or PAP (8) represents a DNA encoding PSA or PAP. BGH pA (13) represents a BGH (bovine growth hormone gene-derived poly-A addition sequence. hTERT enh (15) represents an hTERT enhancer. SV40 enh (16) represents an SV40 enhancer. CMV enh (17) represents a CMV enhancer. The sequences boxed in the sequence shown in the diagram correspond to a BglII restriction enzyme site (10) and an XbaI restriction enzyme site (11), which form a multicloning site. Any of DNAs encoding hIL2, hIL4, hIL7, hGMCSF, mIL4, and mGMCSF can be inserted to the multicloning site between the restriction enzyme sites. In FIGS. 4-1 and 4-2 and FIGS. 5-1 and 5-2, the DNA sequence indicated by (1) represents a portion of the nucleotide sequence of a pIDT-SMART vector serving as the backbone of the gene expression system used. The sequence indicated by (6) represents the sequence of a linker for use in linking RU5′ and the REIC signal peptide-encoding DNA sequence. The sequence indicated by (9) represents the sequence of a linker for use in linking the DNA sequence encoding PSA or PAP and the DNA sequence encoding each cytokine. The sequence indicated by (12) represents a DNA sequence containing three stop codons tag, tga, and taa. The sequence indicated by (18) represents a portion of the nucleotide sequence of a pIDT-SMART vector serving as the backbone of the gene expression system used. Any of DNAs encoding hIL2, hIL4, hIL7, hGMCSF, mIL4, and mGMCSF is inserted to the expression cassettes shown in FIGS. 4-1 and 4-2 and FIGS. 5-1 and 5-2, which are then incorporated into plasmids. The plasmids can be used to produce the fusion proteins of PSA or PAP with any of hIL2, hIL4, hIL7, hGMCSF, mIL4, and mGMCSF. In this context, the REIC signal peptide-encoding DNA sequence was inserted therein such that fusion proteins expressed in large amounts in 293 cells were secreted into a culture solution. In this respect, a sequence encoding the signal peptide of the PSA or PAP protein itself was removed, and instead, the REIC signal peptide-encoding DAN was incorporated thereinto. Nucleotide sequences comprising the hIL2-, hIL4-, hIL7-, hGMCSF-, mIL4-, and mGMCSF-encoding DNAs to be inserted to the expression cassettes are shown in SEQ ID NOs: 3, 4, 5, 6, 7, and 8 and FIGS. 6-1 and 6-2. As shown in FIGS. 6-1 and 6-2, the restriction enzyme sites are located upstream and downstream of any of the sequences represented by SEQ ID NOs: 3 to 8 for insertion to the expression cassettes, while a DNA encoding a 6-histidine amino acid sequence is further located downstream of the DNA encoding each cytokine. The structure of each DNA is shown in the upper area of FIG. 6-1. In this structure diagram, BglII (1) and XbaI (6) represent restriction enzyme sites. Cytokine (2) represents a DNA encoding each cytokine. 6×His tag (4) represents a DNA encoding 6 histidine residues. Stop codon (5) represents a stop codon. The sequence indicated by (3) between the DNA encoding each cytokine and the 6×His tag represents the sequence of a linker used for linking this DNA encoding each cytokine and the 6×His tag.

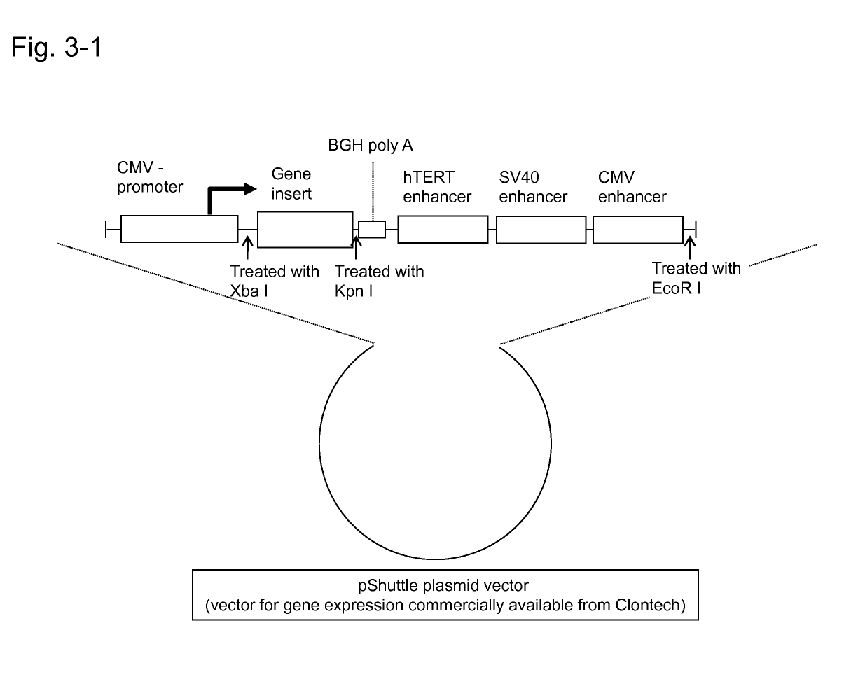

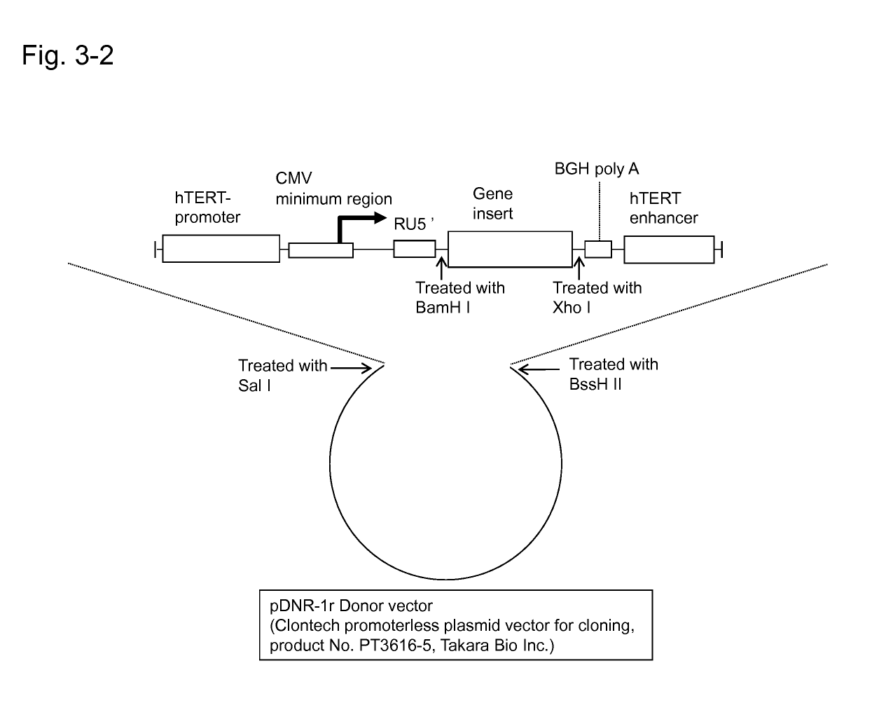

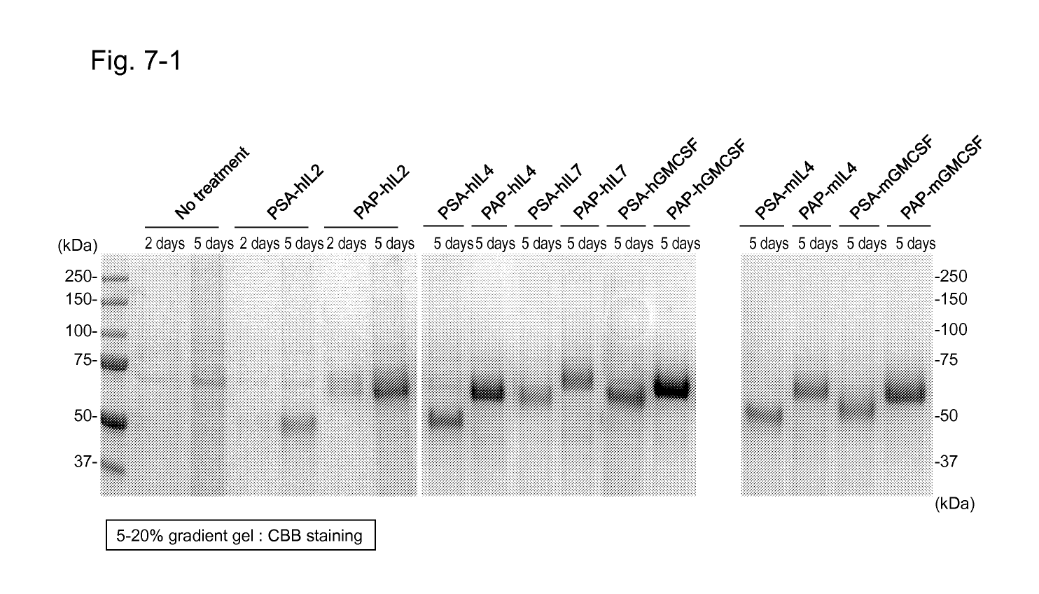

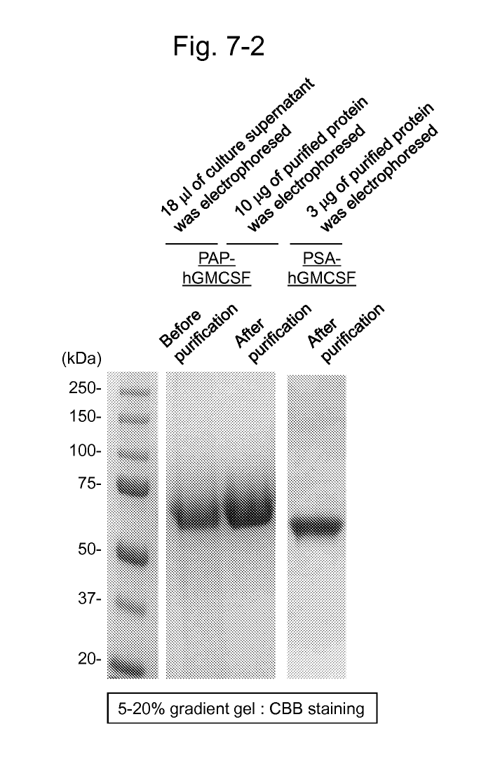

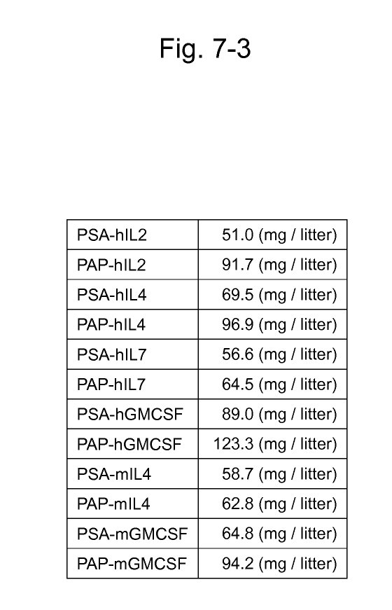

The host cells used for the secretory expression of a total of 12 types of fusion proteins PSA-hIL2, PAP-hIL2, PSA-hIL4, PAP-hIL4, PSA-hIL7, PAP-hIL7, PSA-hGMCSF, PAP-hGMCSF, PSA-mIL4, PAP-mIL4, PSA-mGMCSF, and PAP-mGMCSF were human kidney-derived cells: FreeStyle 293-F cells (Invitrogen Corp.) at the logarithmic growth phase. The cells (30 mL) were inoculated at a concentration of 5 to 6×10⁵ cells/mL to a 125-mL flask and shake-cultured (125 rpm) overnight using Freestyle 293 Expression 1 Media (Invitrogen Corp.) at 37° C. in the presence of 8% CO₂. On the next day, the 293-F cells were concentration-adjusted to 1×10⁶ cells/mL, then inoculated in an amount of 20 mL to a 125-mL flask, and transfected with a mixture of a transfection reagent 293Fectin (Invitrogen Corp.) and a total of 12 types of plasmid DNAs for fusion protein production: plasmid DNAs (6 types) each comprising a DNA construct in which the DNA represented by any of SEQ ID NOs: 3 to 8 was incorporated in 20 μg of the expression cassette represented by SEQ ID NO: 1; and plasmid DNAs (6 types) each comprising a DNA construct in which the DNA represented by any of SEQ ID NOs: 3 to 8 was incorporated in the expression cassette represented by SEQ ID NO: 2. The plasmids used were pIDT-SMART vectors (promoterless plasmid vectors for cloning (Integrated DNA Technologies, Inc. (IDT)). The whole nucleotide sequence of the pIDT-SMART vector is shown in FIG. 6-3 (SEQ ID NO: 9). The cells thus transfected were shake-cultured at 37° C. for 5 days in the presence of 8% CO₂, and the culture supernatant was recovered. An 18 μL aliquot of this culture supernatant was separated using SDS-PAGE. Fusion proteins with each molecular weight (PSA and PAP were glycosylated) were detected by CBB staining. The results are shown in FIG. 7-1.

In order to estimate the amounts of the fusion proteins produced, the PSA-hGMCSF and PAP-hGMCSF fusion proteins secreted into the culture supernatant recovered 5 days after transfection were purified using histidine affinity column chromatography (TALON-Affinity Resin (Clontech Laboratories, Inc.)). The eluates of the purified fusion proteins were separated using SDS-PAGE. The purities of the fusion proteins were confirmed by CBB staining. The results are shown in FIG. 7-2.

In addition, the amounts of 12 types of fusion proteins were determined on the basis of CBB-stained bands of the Bradford method and SDS-PAGE. The amounts of proteins obtained by 1 L culture were calculated from the amounts of the purified proteins in the 20 mL culture. The results are shown in FIG. 7-3.

As shown in FIGS. 7-1, 7-2, and 7-3, 12 types of high-concentration fusion protein solutions were obtained from the supernatant at culture day 5 of the human 293 cells. Particularly, as shown in FIGS. 7-1 and 7-2, very highly pure fusion protein solutions were successfully obtained from the culture supernatant by use of the above-mentioned method for preparing fusion proteins, even without the use of the method for obtaining purified proteins by affinity purification using a His-tag column or the like. Furthermore, use of this method enabled preparation of 12 types of large-volume fusion proteins.

### Example 2 Production of Mouse Models of Human Prostate Cancer

(1) Establishment of PSA-RM9 Cells and PAP-RM9 Cells

An RM-9 cell line was used as a parent line to establish novel cell lines PSA-RM9 cells and PAP-RM9 cells. The PSA-RM9 cells and the PAP-RM9 cells are cell lines persistently expressing human PSA and human PAP, respectively. The parent RM9 cell line is a cancer cell line derived from the prostate of a C57BL/6 mouse and was kindly provided by Prof. Thompson of the Baylor College of Medicine. The RP9 cell line has been confirmed to express neither PSA nor PAP.

The PSA-RM9 cells and the PAP-RM9 cells were established by the following method.

In order to establish the PSA-RM9 cells and the PAP-RM9 cells, plasmids for PSA-RM9 cells and plasmids for PAP-RM9 cells were first constructed. These plasmids were constructed in the same was as in Example 1 by constructing a cassette for foreign gene expression according to the method described in WO2011/062298 and preparing plasmids each comprising the cassette.

Plasmids for PSA-RM9 Cells

The nucleotide sequences of a CMV promoter sequence, a neomycin resistance gene, and an SV40 polyA sequence were incorporated in this order downstream of the sequence of CMV enh to construct plasmids for PSA-RM9 cells. The plasmids are transferred to the cells, so that the transformed cells can express the PSA protein and can also have neomycin resistance.

Plasmids for PAP-RM9 Cells

The nucleotide sequences of a CMV promoter sequence, a neomycin resistance gene, and an SV40 polyA sequence were incorporated in this order downstream of the sequence of CMV enh to construct plasmids for PAP-RM9 cells. The plasmids are transferred to the cells, so that the transformed cells can express the PAP protein and can also have neomycin resistance.

The RM9 cells were seeded over 6-well plates. On the next day, the above-mentioned 2 types of plasmids (plasmids for PSA-RM9 cells and plasmids for PAP-RM9 cells) for stable expression of PSA or PAP were each added thereto at a concentration of 5 μg/well to transfect the RM9 cells using Lipofectamine 2000.

On the next day, the cells were subcultured in 15-cm Petri dishes and cultured in a medium containing Geneticin (G418 Sulfate) (concentration: 500 μg/ml). Approximately 2 weeks later, colonies were picked up, and the cells in the colonies were transferred as clonal lines to 6-well plates. Both of the PSA-RM9 cell line and the PAP-RM9 cell line were each grown into 10 or more clones and preserved in liquid nitrogen.

All of the preserved PSA-RM9 cell clonal lines and the PAP-RM9 cell clonal lines were each confirmed to be clonal lines persistently expressing human PSA and human PAP, respectively, by the measurement of PSA or PAP levels in the culture supernatants. The preserved PSA-RM9 cell clonal lines and the PAP-RM9 cell clonal lines were screened for their respective clonal lines having a high PSA or PAP expression level. These selected clonal lines were used in the production of mouse models of human prostate cancer.

(2) Production of Mouse Models of Human Prostate Cancer

The PSA-RP9 cells or the PAP-RM9 cells (5,000,000 cells/100 μL of PBS) were subcutaneously transplanted to the right thigh of each 8-week-old C57/BL6 male mouse. Four mice were used for the cells of each line. The transplantation day was defined as Day 0. At Days 7 and 14, two mice were sacrificed for each line, and PSA or PAP in the mouse serum was assayed by ELISA. At Day 0, PSA or PAP in the serum of two normal mice was measured for each line by ELISA. The weight of a formed subcutaneous tumor was measured.

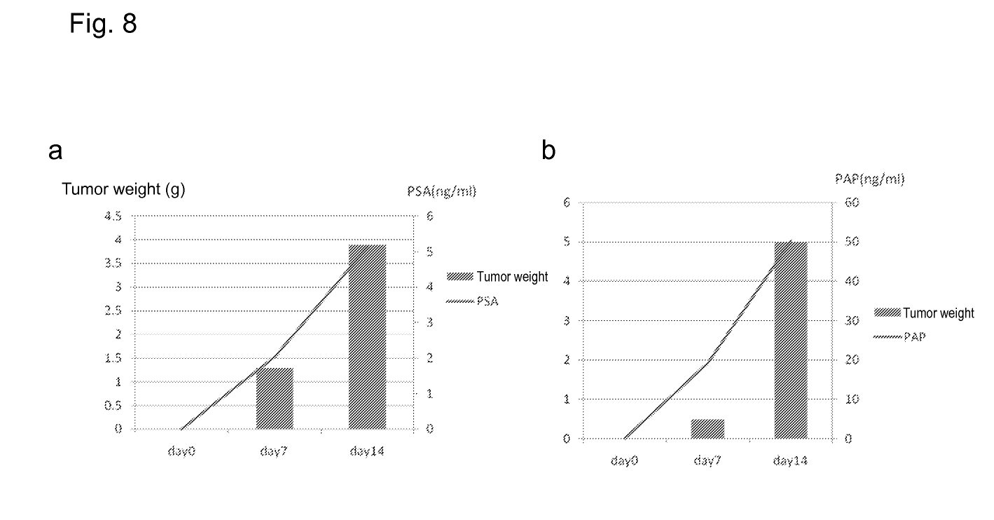

The results are shown in FIG. 8. The value shown in each graph of FIG. 8 represents a mean of the measurement values of two mice. FIG. 8a shows the results about the PSA-RP9 cell-transplanted mice. FIG. 8b shows the results about the PAP-RP9 cell-transplanted mice. As shown in FIG. 8, the PSA or PAP concentration in blood was increased with increase in the size of the tumor. This phenomenon is very similar to the pathological condition of a human prostate cancer patient, demonstrating that the PSA-RP9 cells or the PAP-RP9 cells are useful in the production of mouse models of human prostate cancer.

### Example 3 Treatment Experiment Using Mouse Models of Human Prostate Cancer

The C57/BL6 mouse models of human prostate cancer produced in Example 2 were used to conduct treatment experiments.

Treatment Experiment 1

The mice were divided into the following groups A to E each involving 5 individuals and treated using the fusion proteins prepared in Example 1 as reagents.

Group A (5 individuals) Receiving no reagent
Group B (5 individuals) PSA-mGMCSF: 5 μg (adjusted to 100 μl with PBS)
Group C (5 individuals) PAP-mGMCSF: 5 μg (adjusted to 100 μl with PBS)
Group D (5 individuals) PSA-mGMCSF: 1.25 μg, PSA-mIL4: 1.25 μg, PSA-hIL2: 1.25 μg, and PSA-hIL7: 1.25 μg (adjusted to 100 μl with PBS)
Group E (5 individuals) PAP-mGMCSF: 1.25 μg, PAP-mIL4: 1.25 μg, PAP-hIL2: 1.25 μg, and PAP-hIL7: 1.25 μg (adjusted to 100 μl with PBS)

At Day 0, each reagent was administered to the mice to start the experiment. At Days 2 and 4, the reagent was further administered thereto. At Day 7, the PSA-RM9 cells (left side: 0.8×10⁶ cells) and the PAP-RM9 cells (right side: 1.5×10⁶ cells) were subcutaneously transplanted to both thighs, respectively, of each C57/BL6 mouse (the cells of each line were suspended in 100 μl of PBS and then transplanted). At Day 7, each reagent was intraperitoneally administered (fourth time) thereto. At Days 9, 14, 16, and 18, the reagent was further administered thereto (a total of 9 times). At Day 21, tumor formation was confirmed, and the size of the tumor was measured.

Treatment Experiment 2

The mice were divided into the following groups F and G each involving 5 individuals, and cell reagents were prepared using the fusion proteins prepared in Example 1 and administered from the tail vein of each mouse at Day 0.

Group F (5 individuals) PSA-mGMCSF: 2.5 μg/ml, PSA-mIL4^{.} 2.5 μg/ml, PSA-hIL2: 2.5 μg/ml, and PSA-hIL7: 2.5 μg/ml. The mouse PBMCs (mouse peripheral blood mononuclear cells) were cultured with these fusion proteins for 3 days in an LGM-3 medium and administered once at a dose of PBMCs (1×10⁶ cells/500 μl of PBS) from the tail vein of each mouse.
Group G (5 individuals) PAP-mGMCSF: 2.5 μg/ml, PAP-mIL4: 2.5 μg/ml, PAP-hIL2: 2.5 μg/ml, and PAP-hIL7: 2.5 μg/ml. The mouse PBMCs were cultured with these fusion proteins for 3 days in an LGM-3 medium and administered once at a dose of PBMCs (1×10⁶ cells/500 μl of PBS) from the tail vein of each mouse.

At Day 7, the PSA-RM9 cells (left side: 0.8×10⁶ cells) and the PAP-RM9 cells (right side: 1.5×10⁶ cells) were subcutaneously transplanted to both thighs, respectively, of each C57/BL6 mouse (the cells of each line were suspended in 100 μl of PBS and then transplanted). At Day 21, tumor formation was confirmed, and the size of the tumor was measured. In treatment experiment 2, results about group A of treatment experiment 1 were also used as a control.

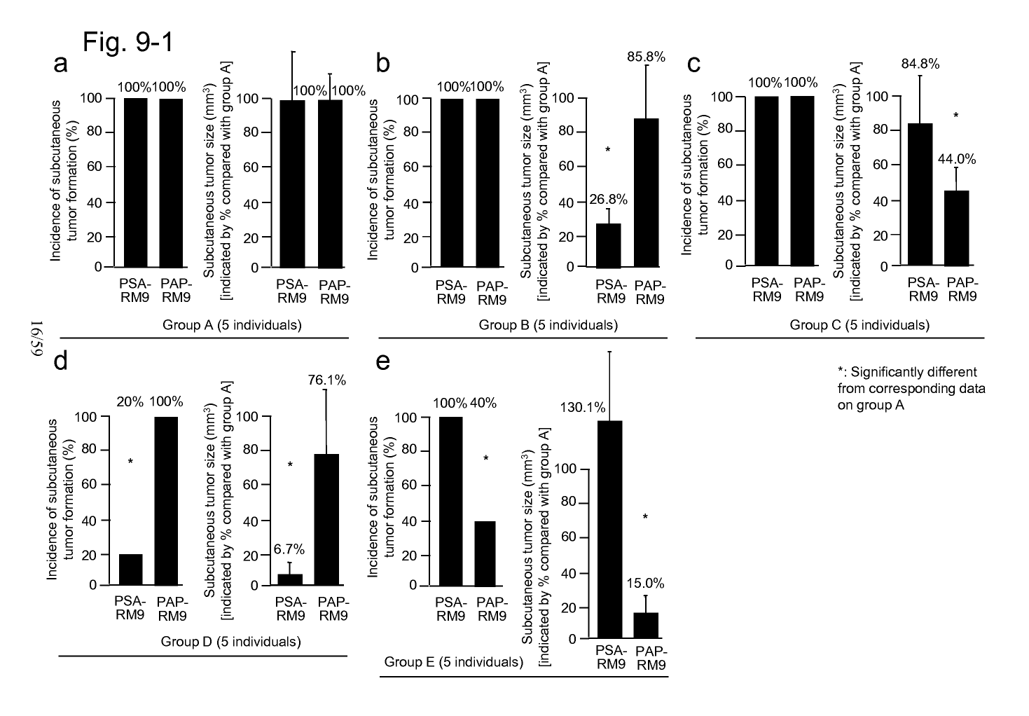

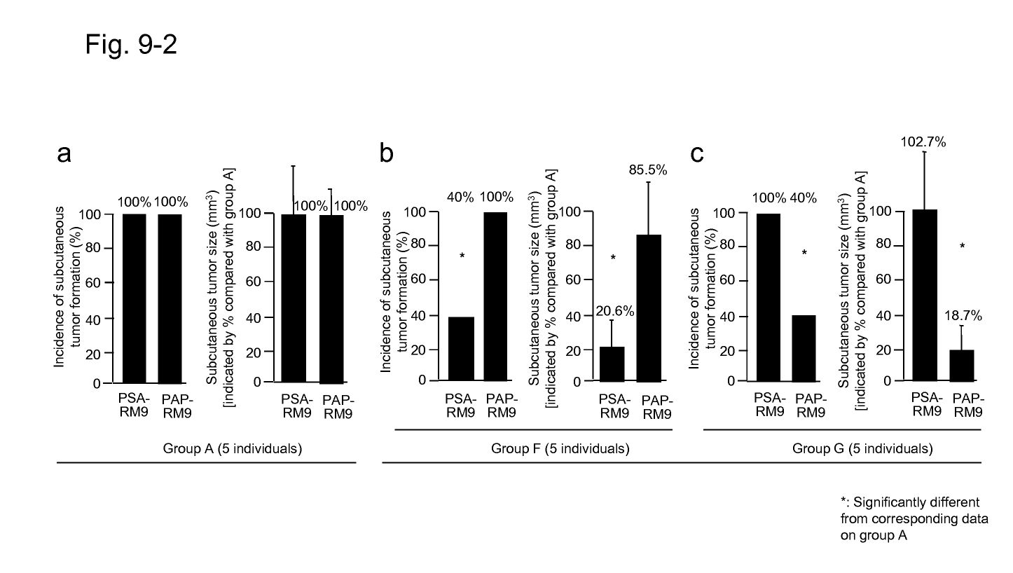

The results are shown in FIGS. 9-1 and 9-2. FIG. 9-1 shows the results of treatment experiment 1. FIGS. 9-1a to 9-1e show the results about groups A to E, respectively. FIG. 9-2 shows the results of treatment experiment 2. FIGS. 9-2a to 9-2c show the results about groups A, F, and G, respectively. “Subcutaneous tumor size (mm³) [indicated by % compared with group A]” was statistically analyzed by the unpaired Student t test between 2 groups to determine a significant difference at p<0.05. Also, “Incidence of subcutaneous tumor formation (%)” was subjected to chi-square test to determine a significant difference at p<0.05.

As shown in FIG. 9-1, the reagents administered to groups B to E were confirmed to have therapeutic effects on the formation or enlargement of a tumor derived from the RM9 cancer cells expressing the same antigen (PSA or PAP protein) as that in the administered reagents.

In treatment experiment 1, significant therapeutic effects (with a significant difference in the inhibition of tumor formation) were confirmed, particularly, in groups D and E compared with groups B and C. This result shows that the administration of two or more fusion proteins in combination enhances anticancer therapeutic effects compared with the administration of a GMCSF-based single agent. This is probably because, by the concurrent administration of two or more fusion proteins to a mouse, the anticancer cytokines respectively contained in the fusion proteins synergistically exert their effects in vivo and can more strongly activate the antitumor immunity than that by the single agent.

As shown in FIG. 9-2, the reagents administered to groups F and G in treatment experiment 2 were confirmed to have significant therapeutic effects on the formation or enlargement of a tumor derived from the RM9 cancer cells expressing the same antigen (PSA or PAP protein) as that in the administered reagents.

### Example 4 Induction of Dendritic Cells by PAP or PSA Fusion Proteins

PSA-mGMCSF and PSA-mIL4 or PAP-mGMCSF and PAP-mIL4 were added in combination to human or mouse monocytes and assayed for the rate of emergence of dendritic cells induced from the monocytes of peripheral blood mononuclear cells (PBMCs).

Human and mouse PBMCs (peripheral blood mononuclear cells) were collected from healthy human and mouse blood by a standard method using Ficoll-Paque centrifugation. The rates of cell recovery were measured by the trypan blue exclusion test to confirm that the viability was 95% or more. In order to prepare monocytes, the human or mouse PBMCs were resuspended in an LGM-3 medium (lymphocyte growth medium-3, serum-free, Lonza Group Ltd.). Plastic-attached cells (incubated at 37° C. for 2 hours in 6-well dishes) were used as monocytes. The obtained human and mouse monocytes were cultured in the presence of the above-mentioned combinations of the fusion proteins (each concentration: 5 μg/ml) or GM-CSF (R&D Systems, Inc.)+IL-4 (R&D Systems, Inc.) (each 2 ng/ml). The cells were observed under a phase contrast microscope.

At each culture day 7, the ratio of dendritic cells to all cells was measured. The microscopically observed morphology of human dendritic cells induced 7 days after the addition of the commercially available hGMCSF and hIL4 proteins to the human monocytes was used as a positive control. Cells confirmed to have a similar form thereto and have dendrites were counted as dendritic cells in each supplemented group. In each fusion protein-supplemented group, the ratio of dendritic cells differentiated by induction to all cells was measured as follows: 7 days after each addition, 100 cells were visually observed in each of a total of 5 random fields of view under direct vision of a microscope at a magnification of ×100, and the number of dendritic cells included in these 100 cells was counted.

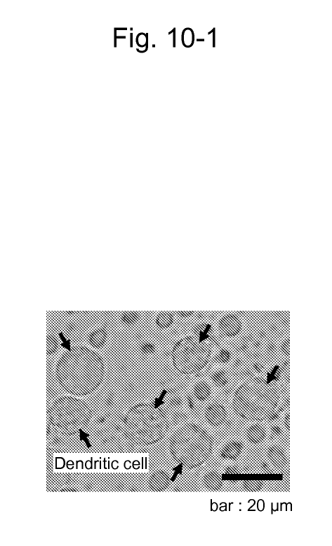

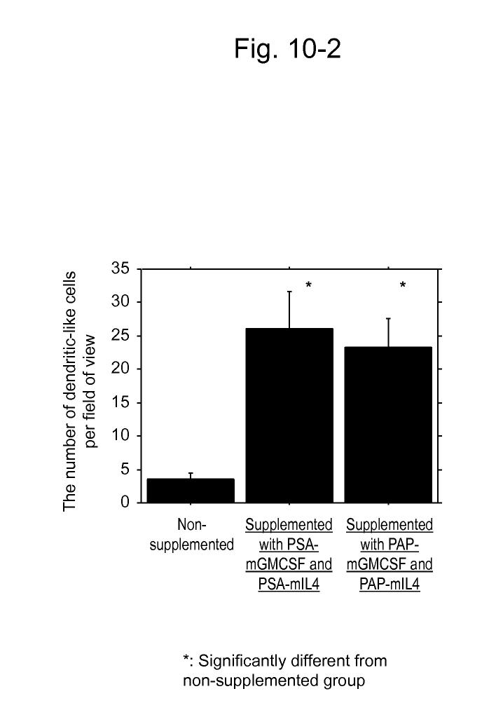

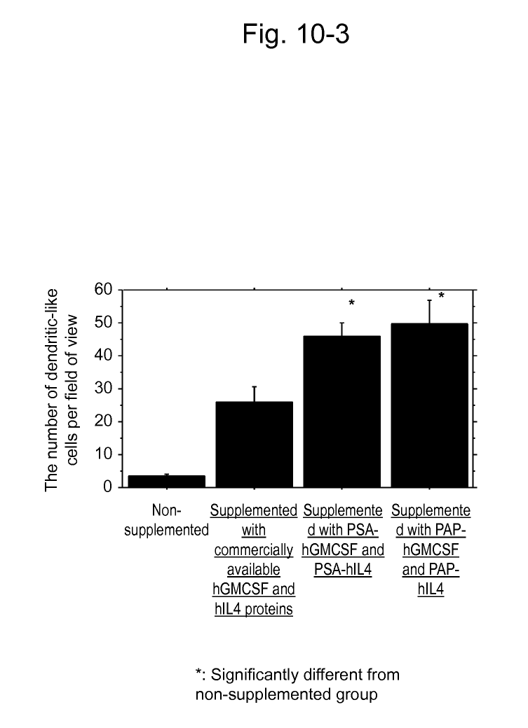

The results are shown in FIGS. 10-1, 10-2, and 10-3. FIG. 10-1 shows the morphology of human dendritic cells induced 7 days after the combined addition of the commercially available hGMCSF and hIL4 proteins to the human PBMCs. In FIG. 10-1, the cells indicated by arrows represent dendritic cells.

FIG. 10-2 shows the rate of emergence of dendritic cells induced from the mouse peripheral blood mononuclear cells (PBMCs) by the addition of PSA-mGMCSF and PSA-mIL4 in combination or PAP-mGMCSF and PAP-mIL4 in combination. As shown in FIG. 10-2, dendritic cell-like cells were observed to be induced at a rate of a few % by culture in the absence of the fusion proteins, whereas dendritic cells were observed to be induced at a rate exceeding 20% by culture in the presence of the fusion proteins in each combination. Specifically, the addition of the fusion proteins was confirmed to produce the expected physiological activity, i.e., the induction of dendritic cells. This result shows that each cytokine (mGMCSF and mIL4) maintains its original functions (dendritic cell induction) even when fused with PSA or PAP.

FIG. 10-3 shows the rate of emergence of dendritic cells induced from the human peripheral blood mononuclear cells (PBMCs) by the addition of PSA-hGMCSF and PSA-hIL4 in combination or PAP-hGMCSF and PAP-hIL4 in combination. As shown in FIG. 10-3, dendritic cell-like cells were observed to be induced at a rate of a few % by culture in the absence of the fusion proteins and at a rate of approximately 25% by culture in the presence of commercially available hGMCSF+hIL4 as a positive control, whereas dendritic cells were observed to be induced at a rate exceeding 45% by culture in the presence of the fusion proteins in each combination. Specifically, the addition of the fusion proteins was confirmed to produce the expected physiological activity, i.e., the induction of dendritic cells. This result shows that each cytokine (mGMCSF and hIL4) maintains its original functions (dendritic cell induction) even when fused with PSA or PAP.

### Example 5 Cell Growth Effects of PSA-hGMCSF and PAP-hGMCSF on TF-1 Cells

Purified PSA-hGMCSF and PAP-hGMCSF were used to analyze their cell growth effects on TF-1 cells by MTT (3-(4,5-di-methylthiazol-2-yl)-2,5-diphenyltetrazolium bromide) assay.

The TF-1 cells were inoculated at a concentration of 10⁴ cells/well to 96-well plates. Each fusion protein was serially diluted at 3-fold dilutions into molar concentrations of 300 pM, 100 pM, 33.3 pM, 11.1 pM, 3.7 pM, 1.2 pM, and 0.41 pM, and further added to the plates. After 3-day culture, MTT assay was conducted using a commercially available cell growth assay reagent. The absorbance at 570 nm was measured to thereby analyze cell growth in each well.

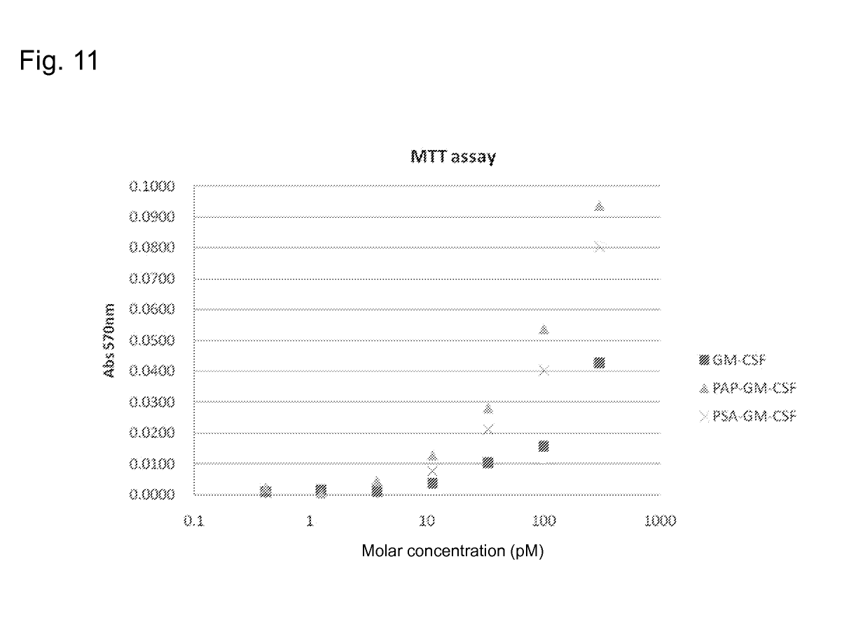

The results are shown in FIG. 11. As shown in FIG. 11, the 2 types of fusion proteins PSA-hGMCSF and PAP-hGMCSF were confirmed to have TF-1 cell growth activity, which is the physiological activity of the hGMCSF protein, at concentrations of 10 pM or higher. This shows that the cytokine (hGMCSF) maintains its original functions even when fused with PSA or PAP.

### Example 6 Purification (Concentration) of PSA- or PAP-Containing Fusion Proteins

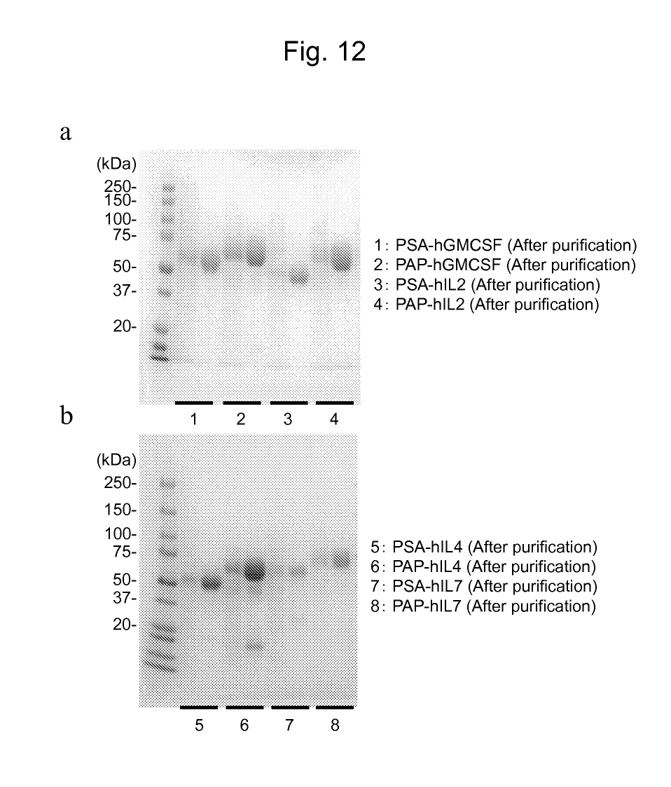

Eight types of fusion proteins PSA-hGMCSF, PAP-hGMCSF, PSA-hIL2, PAP-hIL2, PSA-hIL4, PAP-hIL4, PSA-hIL7, and PAP-hIL7 were produced by the method described in Example 1. Culture supernatants were purified by histidine affinity column chromatography. The eluates of the purified fusion proteins were separated using SDS-PAGE. The purities of the fusion proteins were confirmed by CBB staining. In addition, the amounts of the fusion proteins were determined in the same way as in Example 1 on the basis of CBB-stained bands of the Bradford method and SDS-PAGE. The results of CBB staining are shown in FIG. 12. FIG. 12a shows the results about PSA-hGMCSF, PAP-hGMCSF, PSA-hIL2, and PAP-hIL2. FIG. 12b shows the results about PSA-hIL4, PAP-hIL4, PSA-hIL7, and PAP-hIL7. Each fusion protein was indicated by the results obtained before concentration (left lane) and after concentration (right lane). The 8 types of fusion proteins PSA-hGMCSF, PAP-hGMCSF, PSA-hIL2, PAP-hIL2, PSA-hIL4, PAP-hIL4, PSA-hIL7, and PAP-hIL7 had protein concentrations of 0.52 mg/ml, 0.7 mg/ml, 0.31 mg/ml, 0.68 mg/ml, 0.53 mg/ml, 1.17 mg/ml, 0.13 mg/ml, and 0.23 mg/ml, respectively.

The fusion proteins of the present invention were obtained at clinically available levels of very high concentrations. Sipuleucel-T (Provenge®) is used at a protein concentration of 10 μg/ml to be added for cell culture, whereas the fusion protein group of the present invention can be diluted and added to cells at a concentration higher than the concentration of 10 μg/ml used in Sipuleucel-T for cell culture.

### Example 7 Production of PSMA-Containing Fusion Proteins and Analysis of their Growth Effects on TF-1

(1) Production of PSMA-hGMCSF Fusion Protein

A fusion protein of PSMA (prostate-specific membrane antigen) and human GMCSF (hGMCSF) was produced.

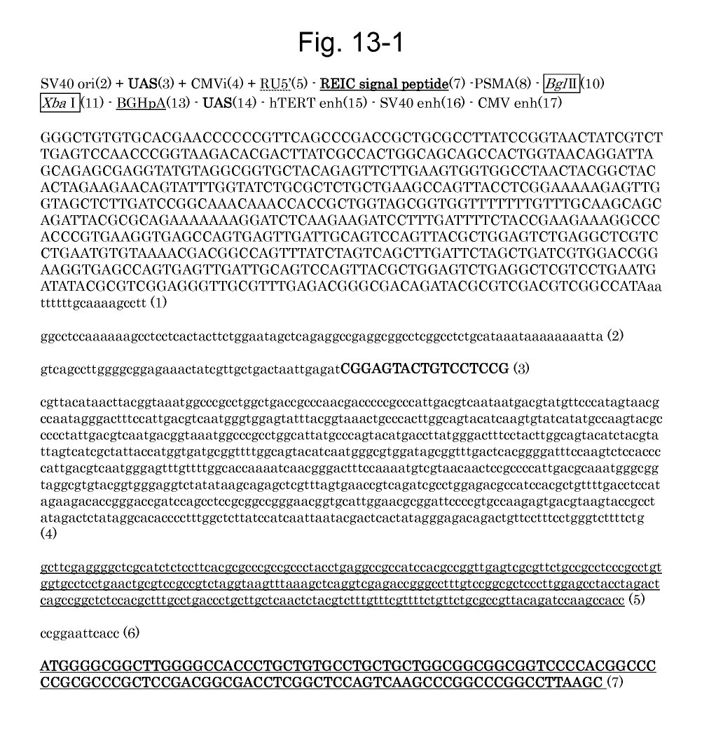

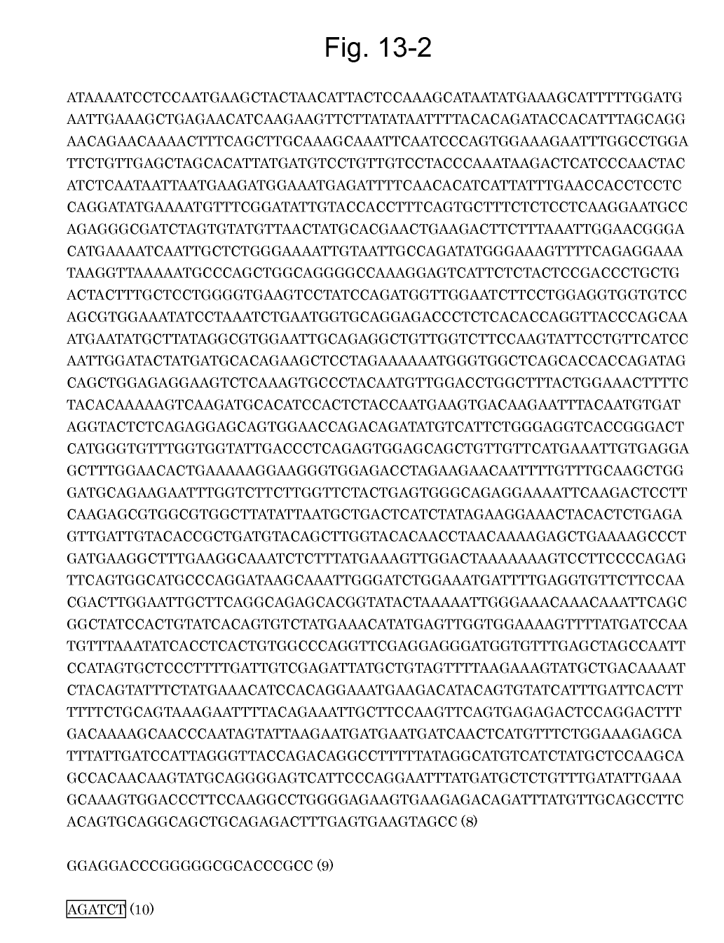

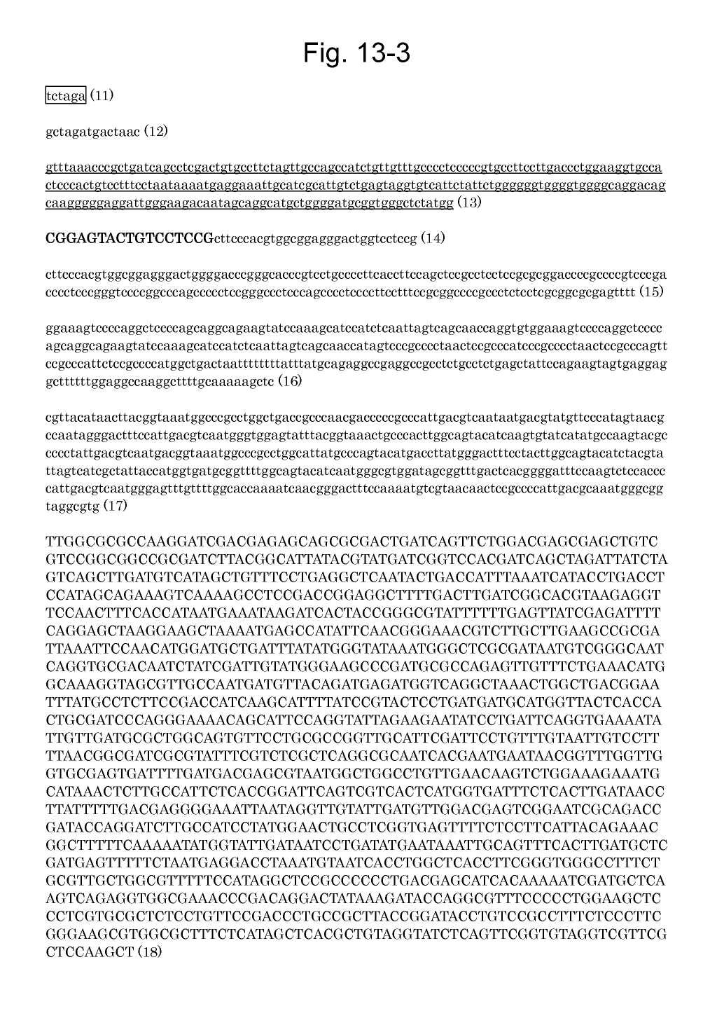

In this Example, the expression cassette shown in FIGS. 13-1, 13-2, and 13-3 (its sequence is shown in SEQ ID NO: 10) was used. The sequence shown in FIG. 13-2 is a sequel to the sequence shown in FIG. 13-1. The sequence shown in FIG. 13-3 is a sequel to the sequence shown in FIG. 13-2. A DNA encoding hGMCSF is inserted to between the sequence shown in FIG. 13-2 and the sequence shown in FIG. 13-3 by use of restriction enzyme sites. The meanings of the sequences shown in FIGS. 13-1, 13-2, and 13-3 and each element are the same as in the PSA-containing expression cassette (FIGS. 4-1 and 4-2) and the PAP-containing expression cassette (FIGS. 5-1 and 5-2) of Example 1 except that PSMA represents a sequence encoding PSMA. This PSMA used was the extracellular region of PSMA. A nucleotide sequence comprising the hGMCSF-encoding DNA to be inserted to the expression cassette is shown in SEQ ID NO: 6.

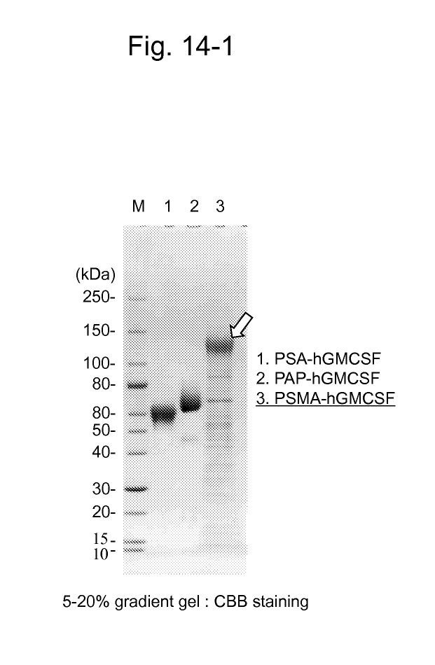

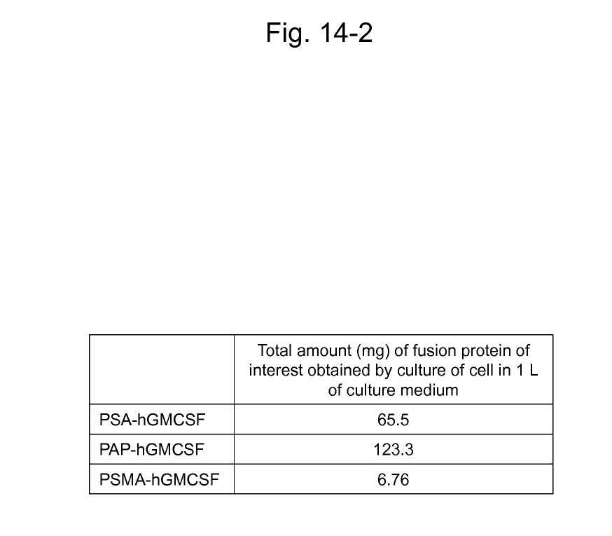

The fusion protein of PSMA and hGMCSF was prepared and purified in the same way as the method described in Example 1. The fusion protein (3 μg) thus obtained by purification was subjected to SDS-PAGE. The purity of the fusion protein was confirmed by CBB staining. The results are shown in FIG. 14-1. FIG. 14-1 shows the results about PSA-hGMCSF and PAP-GMCSF produced in Example 1 and PMSA-hGMCSF produced in this Example.

The amount of the fusion protein was determined on the basis of CBB-stained bands of the Bradford method and SDS-PAGE. The amount of a protein obtained by 1 L culture was calculated from the amount of the purified protein in the 20 mL culture). The results are shown in FIG. 14-2.

As shown in FIGS. 14-1 and 14-2, a highly pure PSMA-hGMCSF fusion protein solution was successfully obtained from the culture supernatant. Since PSMA is a prostate cancer antigen, PMSA-hGMCSF can be used in cancer immunotherapy for prostate cancer.

(2) Analysis of Growth Effects of PSMA-hGMCSF Fusion Protein on TF-1 Cells

PSMA-hGMCSF purified in the same way as the method described in Example 5 was used to analyze its cell growth effects on TF-1 cells by MTT (3-(4,5-di-methylthiazol-2-yl)-2,5-diphenyltetrazolium bromide) assay.

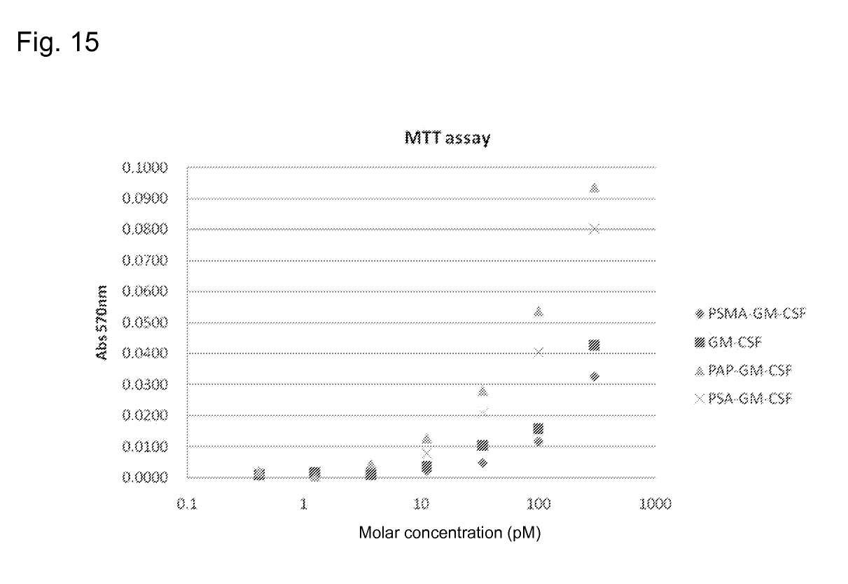

The results are shown in FIG. 15. FIG. 15 also shows the results about PAP-hGMCSF, PSA-hGMCSF, and GMCSF (control). As shown in FIG. 15, the cytokine (hGMCSF) in the PSMA-hGMCSF fusion protein obtained by the fusion between hGMCSF and PSMA maintained its original function, as in PAP-hGMCSF and PSA-hGMCSF. This shows that PSMA-hGMCSF can be effectively used in cancer immunotherapy for prostate cancer.

### Example 8 Preparation of MAGEA4- or CD147-Containing Fusion Proteins

Fusion proteins of MAGEA4 (melanoma-associated antigen 4) or CD147 with human IL2 (hIL2), human IL4 (hIL4), human IL7 (hIL7), human GMCSF (hGMCSF), mouse IL4 (mIL4), or mouse GMCSF (mGMCSF) were produced.

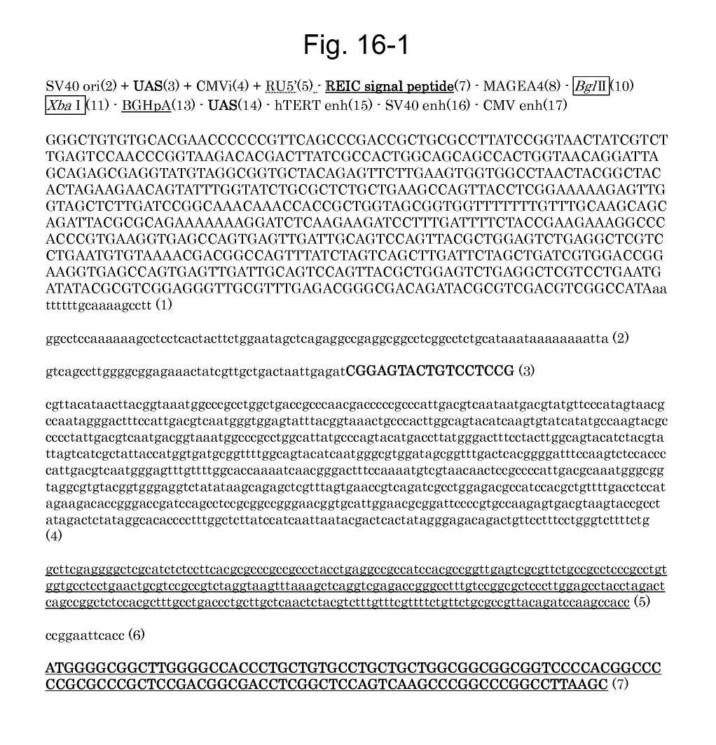

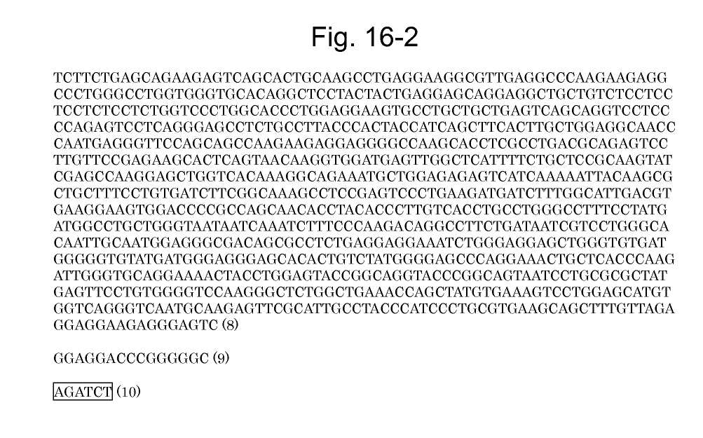

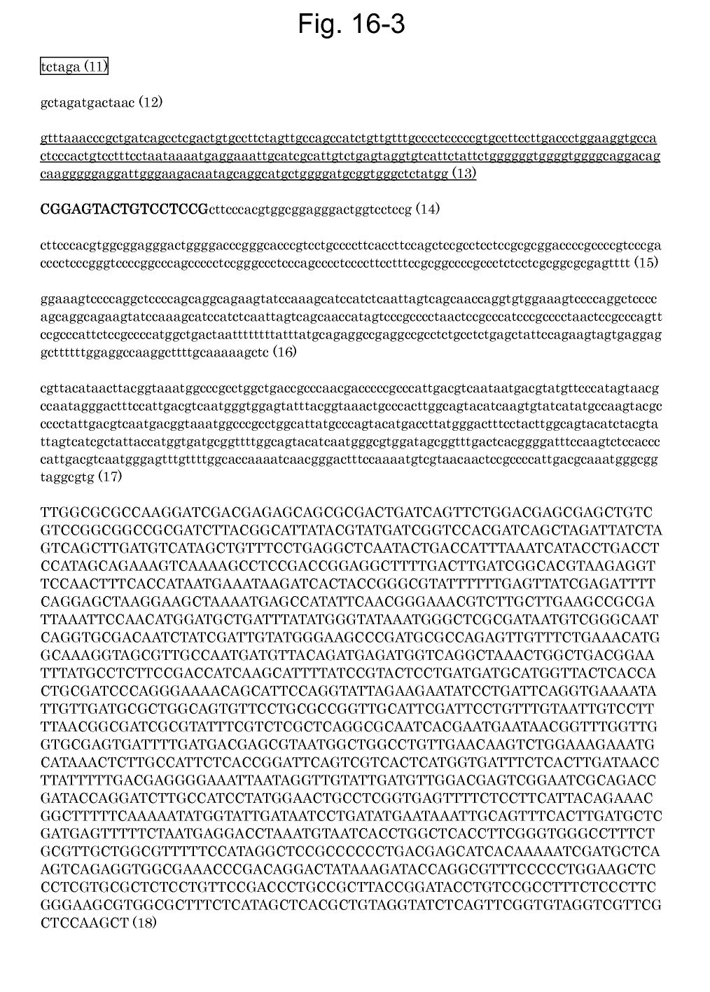

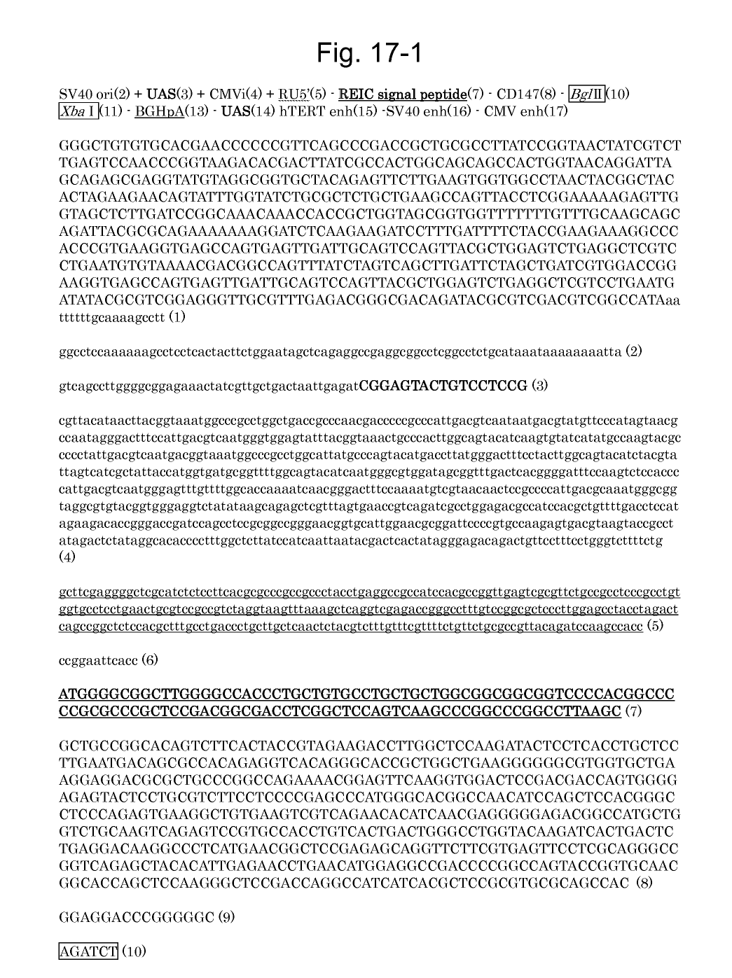

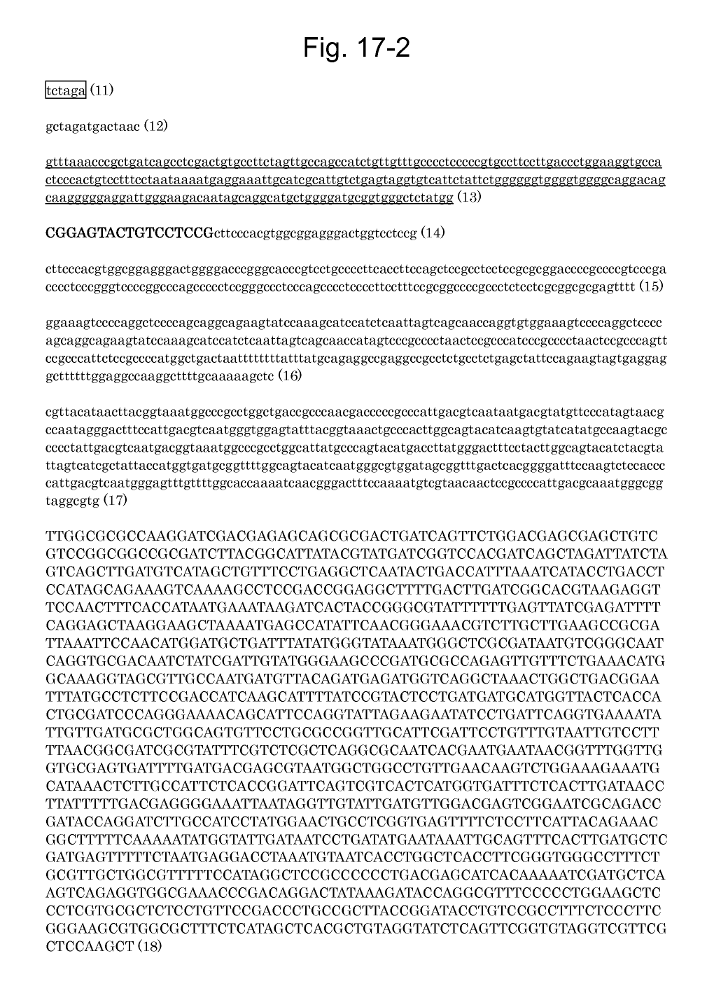

The production was carried out in the same way as the method described in Example 1. In this Example, the expression cassettes shown in FIGS. 16-1, 16-2, and 16-3 and FIGS. 17-1 and 17-2 (their sequences are shown in SEQ ID NOs: 11 and 12, respectively) were used. The sequence shown in FIG. 16-2 is a sequel to the sequence shown in FIG. 16-1. The sequence shown in FIG. 16-3 is a sequel to the sequence shown in FIG. 16-2. A DNA encoding each cytokine is inserted to between the sequence shown in FIG. 16-2 and the sequence shown in FIG. 16-3 by use of restriction enzyme sites. Likewise, the sequence shown in FIG. 17-2 is a sequel to the sequence shown in FIG. 17-1. A DNA encoding each cytokine is inserted to between the sequence shown in FIG. 17-1 and the sequence shown in FIG. 17-2 by use of restriction enzyme sites. The meanings of the sequences shown in FIGS. 16-1, 16-2, and 16-3 and FIGS. 17-1 and 17-2 and each element are the same as in the PSA-containing expression cassette (FIGS. 4-1 and 4-2) and the PAP-containing expression cassette (FIGS. 5-1 and 5-2) of Example 1 except that MAGEA4 represents a sequence encoding MAGEA4 (FIGS. 16-1, 16-2, and 16-3) and CD147 represents a sequence encoding CD147 (FIGS. 17-1 and 17-2). This CD147 used was the extracellular region of CD147. Nucleotide sequences comprising the hIL2-, hIL4-, hIL7-, hGMCSF-, mIL4-, and mGMCSF-encoding DNAs to be inserted to the expression cassettes are shown in SEQ ID NOs: 3, 4, 5, 6, 7, and 8 and FIGS. 6-1 and 6-2.

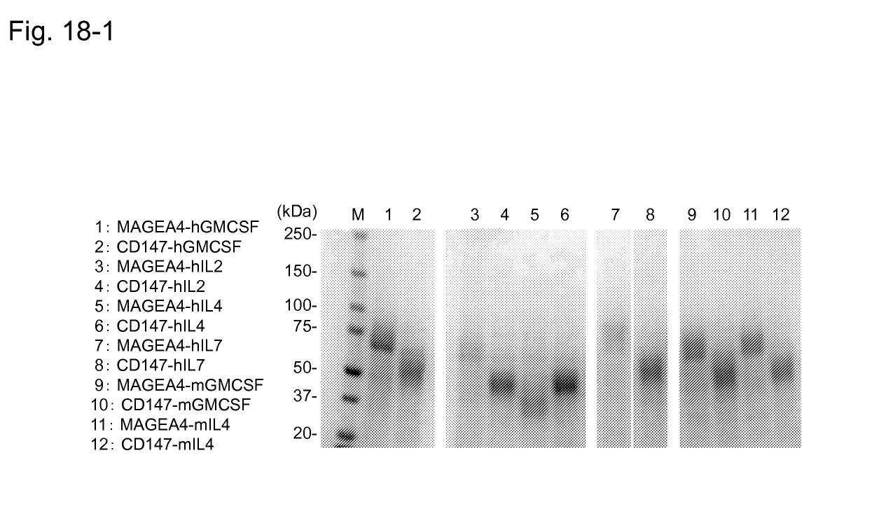

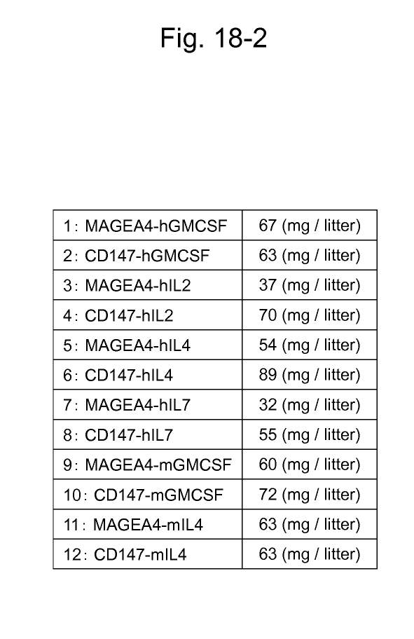

The fusion proteins purified in the same way as the method described in Example 1 were subjected to SDS-PAGE. The purities of the fusion proteins were confirmed by CBB staining. The results are shown in FIG. 18-1. In addition, the amounts of the fusion proteins were determined in the same way as in Example 1 on the basis of CBB-stained bands of the Bradford method and SDS-PAGE. The amounts of proteins obtained by 1 L culture were calculated from the amounts of the purified proteins in the 20 mL culture. The results are shown in FIG. 18-2. As shown in FIGS. 18-1 and 18-2, the 12 types of fusion proteins (MAGEA4-hGMCSF, CD147-hGMCSF, MAGEA4-hIL2, CD147-hIL2, MAGEA4-hIL4, CD147-hIL4, MAGEA4-hIL7, CD147-hIL7, MAGEA4-mGMCSF, CD147-mGMCSF, MAGEA4-mIL4, and CD147-mIL4) were obtained at high concentrations in the supernatant at culture day 5 using human 293 cells.

Since the MAGEA4 and CD147 proteins function as cancer antigens in cancer cells of a wide range of cancer types and serve as markers of cancer-targeting treatment, the fusion proteins of the MAGEA4 or CD147 protein with various cytokines can be used in cancer immunotherapy for a wide range of cancer types.

### Example 9 Preparation of CEA1- or CEA2-Containing Fusion Proteins

Fusion proteins of CEA1 or CEA2 with human IL2 (hIL2), human IL4 (hIL4), human IL7 (hIL7), human GMCSF (hGMCSF), mouse IL4 (mIL4), or mouse GMCSF (mGMCSF) were produced.

The human carcinoembryonic antigen (CEA, CD66e) is a glycoprotein (sugar content: 50 to 60%; 702 amino acids (including a signal peptide); the gene is located on 19q13.2) of approximately 180 kDa that is found mainly in gastrointestinal adenocarcinomas including colon cancer. Its expression is not specific for gastrointestinal cancer and is used as a marker of epithelial tumors in various organs such as the lung and mammary glands. CEA is also seen, albeit slightly, in the normal mucosa of the large intestine and found reactive with the surface of the duct of the gland, but strongly reacts with the cytoplasms of cancer cells.

There exists a protein group called human NCA (non-specific cross-reacting antigen) or human PSG (pregnancy-specific glycoprotein), which exhibits very high homology to the sequence of the human CEA amino acid residues. These protein groups and CEA are collectively referred to as the CEA family. Their genes including the CEA gene are located closely to each other on the chromosome 19q13.1-q13.3. These members of the CEA family function as adhesion molecules in terms of physiological activity. These protein groups of the CEA family are also expressed in diverse cancers.

This Example was conducted to show the usefulness of cancer immunotherapy with the protein groups of the CEA family as target antigens.

The non-specific cross-reacting antigen (NCA, CD66c) is an adhesion molecule of approximately 37 kDa (as a precursor) that is composed of 344 (including a signal peptide) amino acids and also expressed in granulocytic leukocytes, etc. This protein constitutes the family with CEA. As shown in the next page, NCA and CEA have high homology (underlined) between the sequences of their amino acid residues as if NCA is a portion of CEA, and tend to cause immunological cross-reaction.

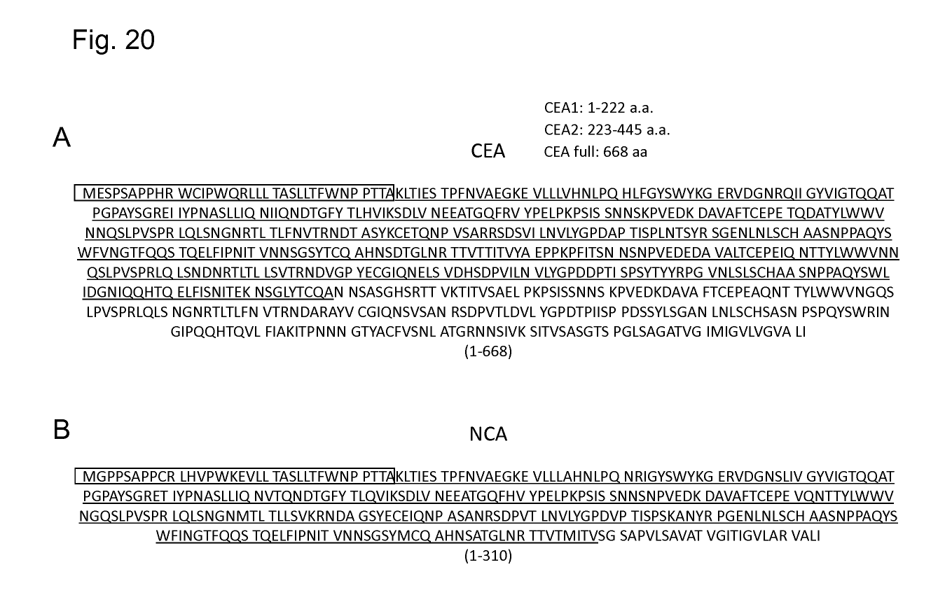

In order to comprehensively use these protein groups of the CEA family including NCA as target antigens for immunotherapy, underlined 222-amino acid and 223-amino acid moieties in the amino acid sequence (SEQ ID NO: 15; the nucleotide sequence of a DNA encoding the amino acid sequence is shown in SEQ ID NO: 14) of the CEA protein composed of 668 amino acids shown in FIG. 20A were divided into “CEA1” (SEQ ID NO: 17; the nucleotide sequence of a DNA encoding the amino acid sequence is shown in SEQ ID NO: 16) and “CEA2” (SEQ ID NO: 19; the nucleotide sequence of a DNA encoding the amino acid sequence is shown in SEQ ID NO: 18), respectively, and used in this Example to prepare CEA1 and CEA2 fusion proteins. The amino acid sequence of residues 1 to 34 in FIG. 20A corresponds to a signal peptide sequence (boxed), which is removed in the production of the fusion proteins of the present invention. FIG. 20B shows the amino acid sequence of NCA (SEQ ID NO: 20). The amino acid sequence of NCA homologous to the whole sequence moiety of CEA1 and CEA2 is underlined in FIG. 20B (SEQ ID NO: 20). The amino acid sequence of residues 1 to 34 in FIG. 20B corresponds to a signal peptide sequence (boxed), which is removed in the production of the fusion proteins of the present invention.

The production was carried out in the same way as the method described in Example 1. Specifically, any of DNAs encoding CEA1 and CEA2 was inserted to the gene insert moiety indicated by PSA in the expression cassette having the sequence shown in FIGS. 4-1 and 4-2. A DNA encoding each cytokine is inserted to between the sequence shown in FIG. 4-1 and the sequence shown in FIG. 4-2 by use of restriction enzyme sites.

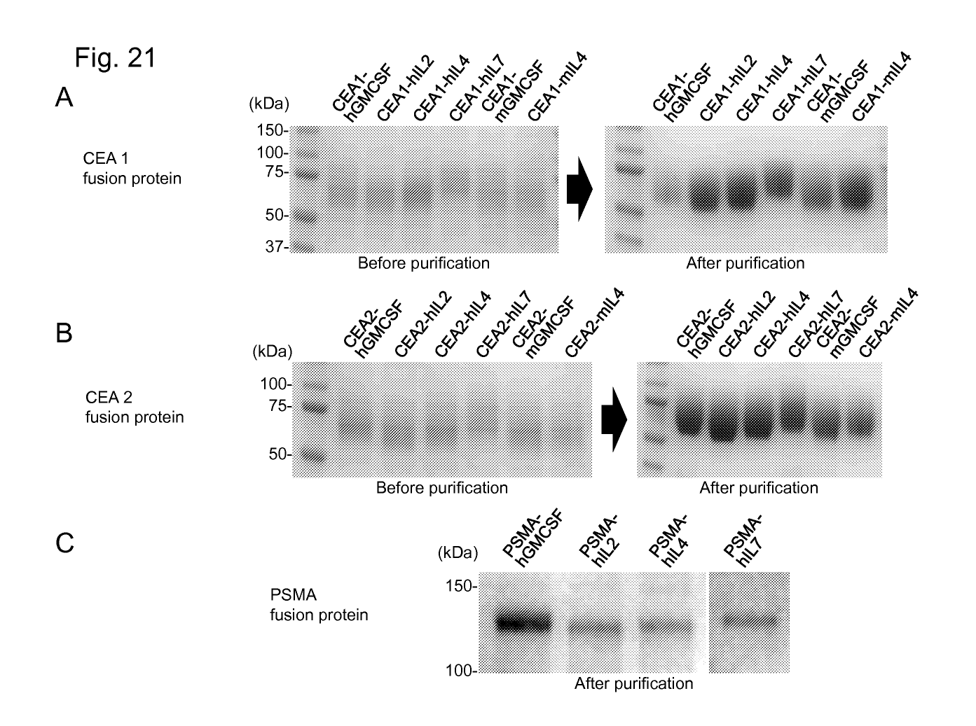

The fusion proteins purified in the same way as the method described in Example 1 were subjected to SDS-PAGE. The purities of the fusion proteins were confirmed by CBB staining. Also, the purities of these fusion proteins were confirmed before purification. The results are shown in FIG. 21. FIG. 21A shows the results about the fusion proteins of CEA1 and each cytokine before and after purification. FIG. 21B shows the results about the fusion proteins of CEA2 and each cytokine before and after purification.

The bold-faced amino acid sequence moiety in the CEA protein divided into “CEA1” and “CEA2” for use as mentioned above exhibits high homology not only to NCA but to many members of the protein groups of the CEA family. The CEA1 and CEA2 fusion protein (cytokine) groups can therefore be combined to thereby achieve comprehensive treatment with the protein groups of the CEA family as target antigens. The division of this site into CEA1 and CEA2 is also important for the purpose of reducing the sizes of expressed proteins in order to increase the yields of the obtained fusion proteins.

The CEA1 and CEA2 fusion proteins thus designed and prepared can be used alone or in combination to carry out immunotherapy against cancers and other diseases targeting a wide range of protein groups of the CEA family.

### Example 10 Preparation of PMSA-Containing Fusion Proteins (Part 2)

Fusion proteins of PMSA with human IL2 (hIL2), human IL4 (hIL4), human IL7 (hIL7), or human GMCSF (hGMCSF) were produced.

The production was carried out in the same way as the method described in Example 1. Specifically, the fusion protein of PSMA and human GMCSF (hGMCSF) was produced in Example 7, while the fusion proteins of PMSA with human IL2 (hIL2), human IL4 (hIL4), human IL7 (hIL7), or human GMCSF (hGMCSF) were produced in the same way as above except that the cytokine was changed.

The fusion proteins purified in the same way as the method described in Example 1 were subjected to SDS-PAGE. The purities of the fusion proteins were confirmed by CBB staining. The results are shown in FIG. 21C.

### Example 11 Measurement of Concentrations of Various Fusion Proteins after Purification

The concentrations of various fusion proteins produced by the method of the present invention were measured after purification.

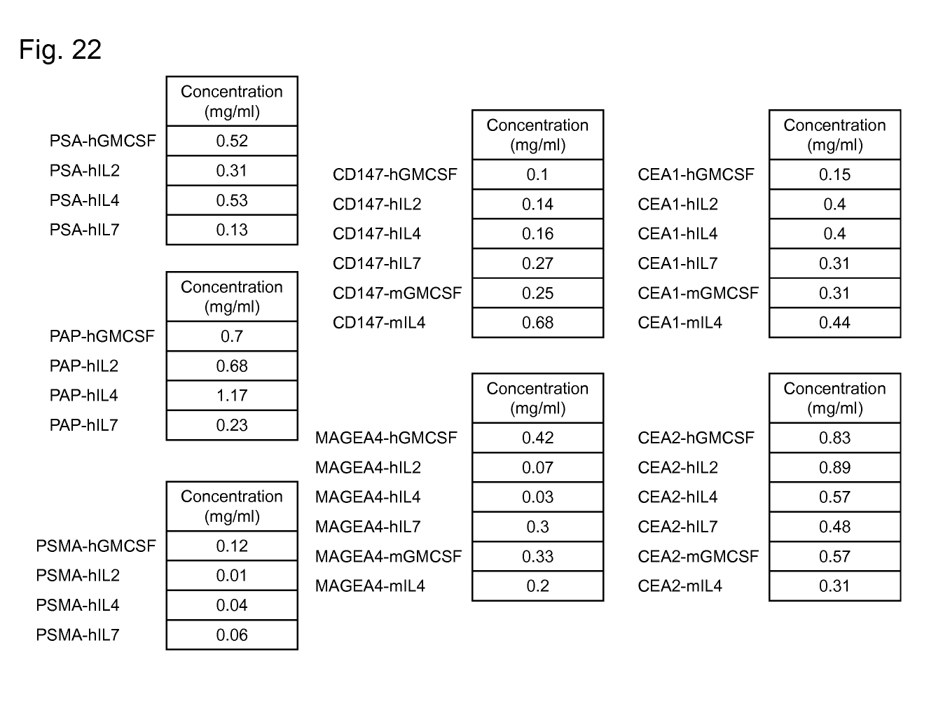

The amounts of various fusion proteins were determined on the basis of CBB-stained bands of the Bradford method and SDS-PAGE. The amounts of proteins obtained by 1 L culture were calculated from the amounts of the purified proteins in the 20 mL culture. The results are shown in FIG. 22.

Purified fusion protein solutions (4 ml each) having the concentrations mentioned above can be obtained by the affinity purification of 125 ml of a culture supernatant containing various fusion proteins using a His-tag column. Furthermore, various fusion proteins can be efficiently (with more emphasis placed on yields) concentrated by use of a protein ultrafiltration method generally used.

### Example 12 Induction of Dendritic Cells Using Various Fusion Proteins and Combinations of Fusion Proteins

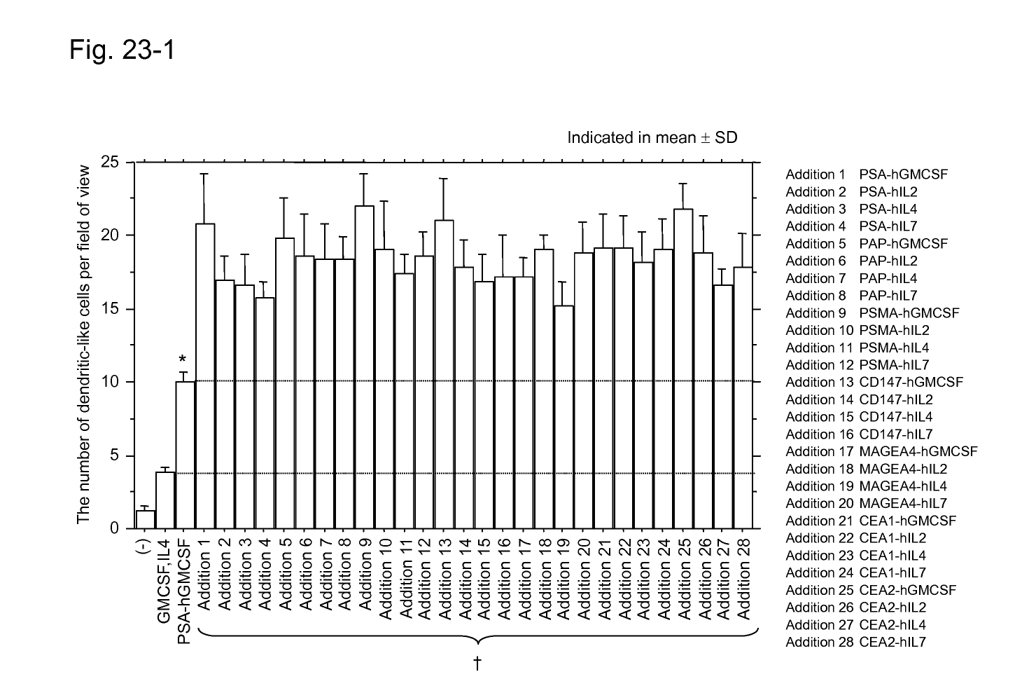

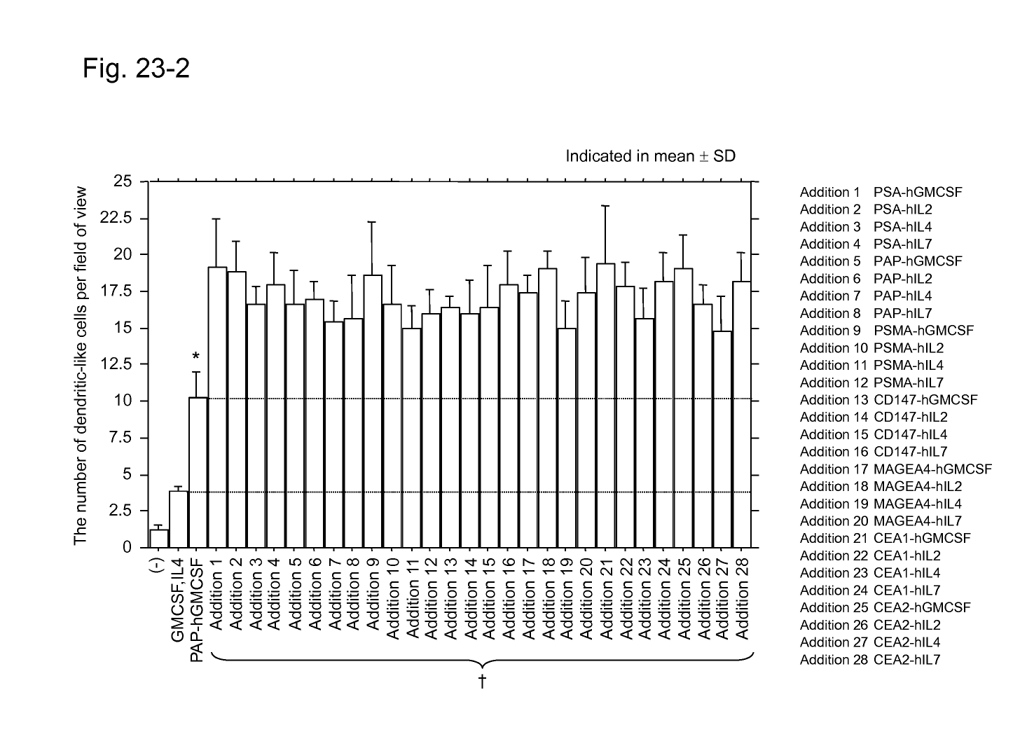

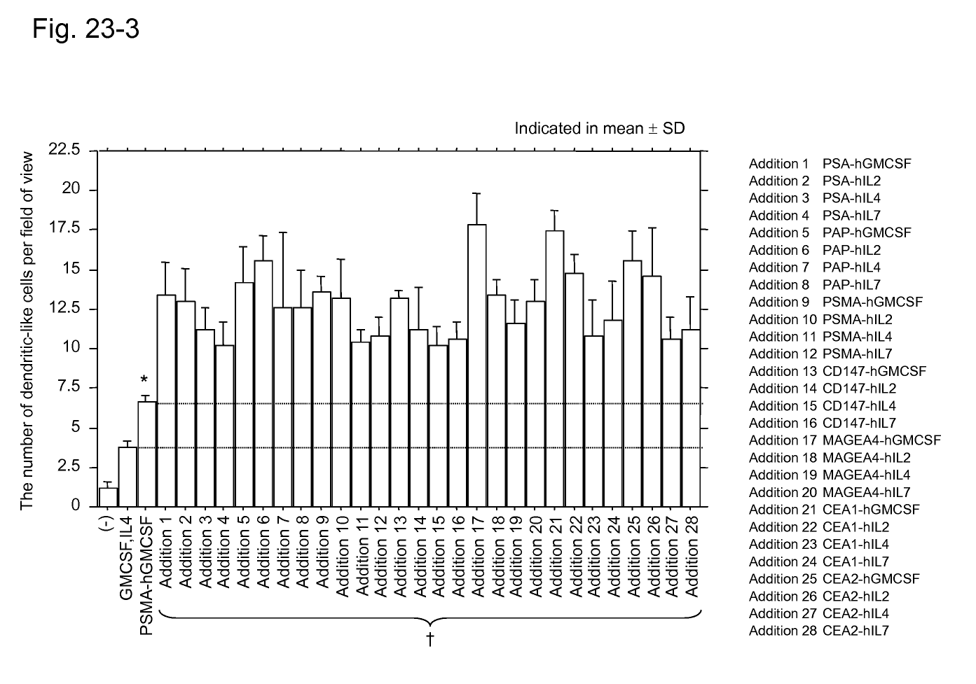

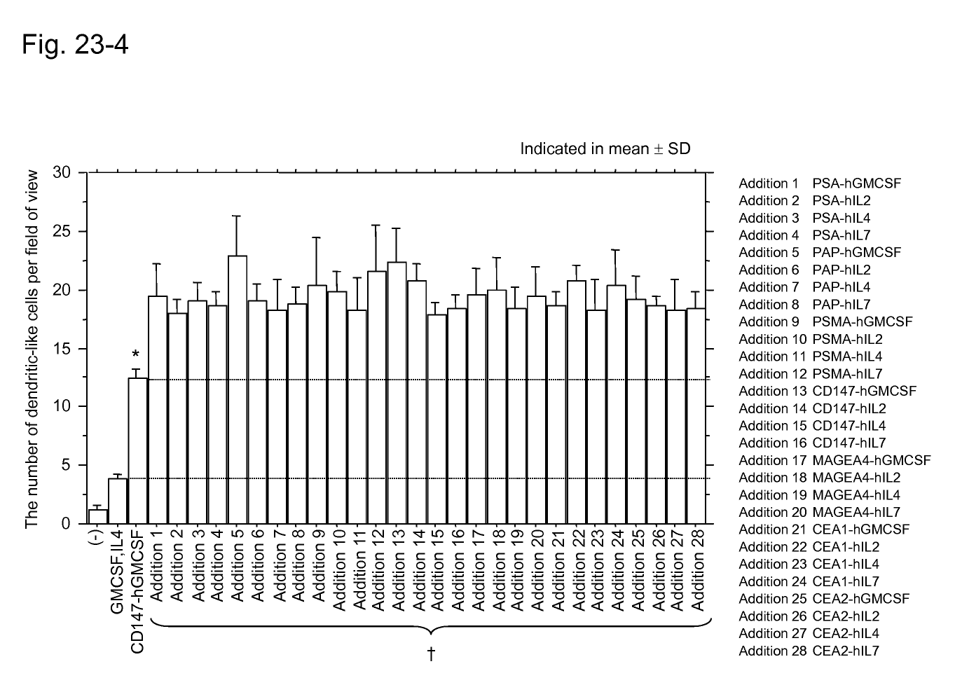

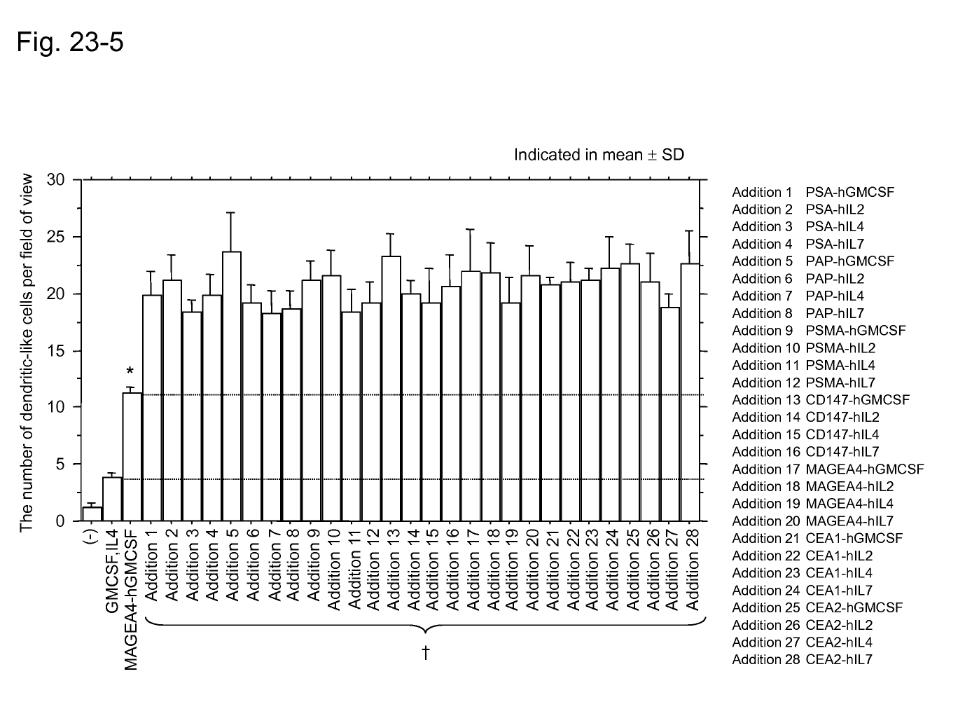

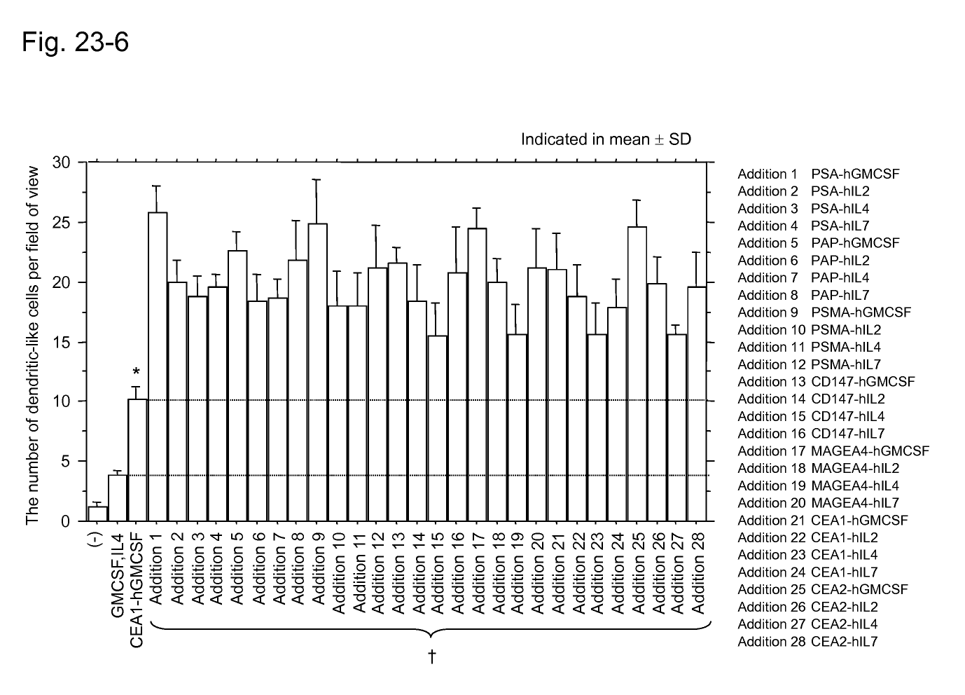

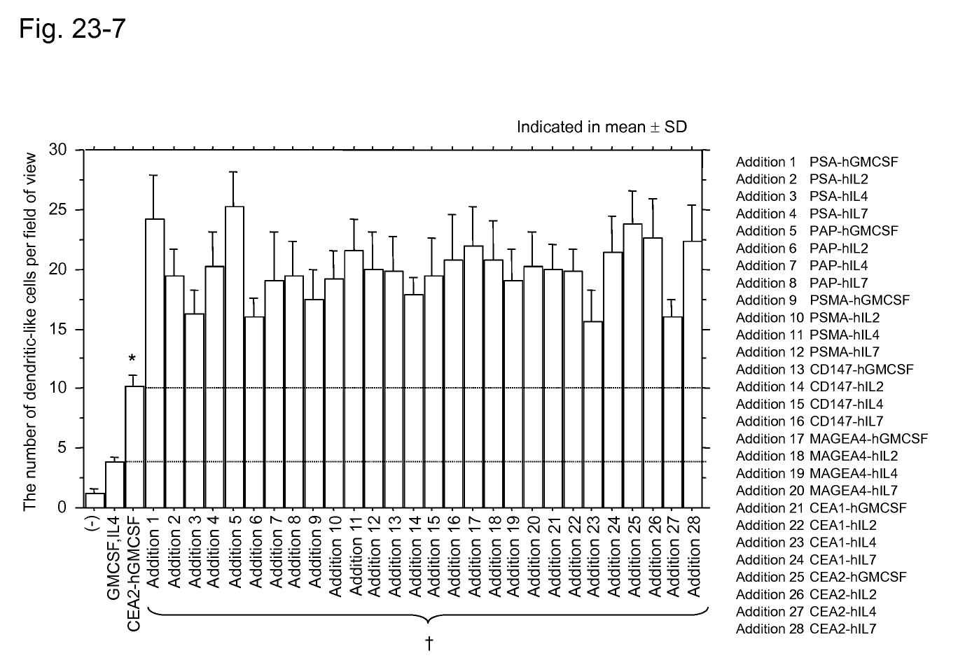

(1) Dendritic cells were induced by a modification of the method described in Example 4 using PSA-hGMCSF in combination with various fusion proteins. Specifically, CD14-positive monocytes of human peripheral blood were cultured for 3 days with commercially available cytokines hGMCSF and hIL4 (both added at a concentration of 2 ng/ml) ([hGMCSF,hIL4] group), the fusion protein PSA-hGMCSF (added at a concentration of 1 μg/ml) ([PSA-hGMCSF] group), or PSA-hGMCSF further combined with 1 type of fusion protein (all added at a concentration of 1 μg/ml). The rate of emergence of dendritic cells was measured. FIG. 23-1 shows the rate of emergence of dendritic cells induced in each treatment group.

As shown in FIG. 23-1, use of PSA-hGMCSF alone (indicated by * in the diagram) significantly increased the rate of emergence of dendritic cells compared with the [hGMCSF,hIL4] group. This means that use of the fusion protein PSA-hGMCSF alone was more useful than use of unfused hGMCSF and hIL4. Use of PSA-hGMCSF further combined with 1 type of fusion protein (indicated by †) significantly increased the rate of emergence of dendritic cells compared with the [PSA-hGMCSF] group. This means that use of PSA-hGMCSF further combined with 1 type of fusion protein was more useful than use of the fusion protein PSA-hGMCSF alone.

(2) In the same way as in (1), dendritic cells were induced using PAP-hGMCSF in combination with various fusion proteins. PSA-hGMCSF was combined with various fusion proteins in (1), while PAP-hGMCSF was combined with various fusion proteins in (2). FIG. 23-2 shows the rate of emergence of dendritic cells induced in each treatment group.

As shown in FIG. 23-2, use of PAP-hGMCSF alone (indicated by * in the diagram) significantly increased the rate of emergence of dendritic cells compared with the [hGMCSF,hIL4] group. This means that use of the fusion protein PAP-hGMCSF alone was more useful than use of unfused hGMCSF and hIL4. Use of PAP-hGMCSF further combined with 1 type of fusion protein (indicated by †) significantly increased the rate of emergence of dendritic cells compared with the [PAP-hGMCSF] group. This means that use of PAP-hGMCSF further combined with 1 type of fusion protein was more useful than use of the fusion protein PAP-hGMCSF alone.

(3) In the same way as in (1), dendritic cells were induced using PSMA-hGMCSF in combination with various fusion proteins. PSA-hGMCSF was combined with various fusion proteins in (1), while PSMA-hGMCSF was combined with various fusion proteins in (3). FIG. 23-3 shows the rate of emergence of dendritic cells induced in each treatment group.

As shown in FIG. 23-3, use of PSMA-hGMCSF alone (indicated by * in the diagram) significantly increased the rate of emergence of dendritic cells compared with the [hGMCSF,hIL4] group. This means that use of the fusion protein PSMA-hGMCSF alone was more useful than use of unfused hGMCSF and hIL4. Use of PSMA-hGMCSF further combined with 1 type of fusion protein (indicated by †) significantly increased the rate of emergence of dendritic cells compared with the [PSMA-hGMCSF] group. This means that use of PSMA-hGMCSF further combined with 1 type of fusion protein was more useful than use of the fusion protein PSMA-hGMCSF alone.

(4) In the same way as in (1), dendritic cells were induced using CD147-hGMCSF in combination with various fusion proteins. PSA-hGMCSF was combined with various fusion proteins in (1), while CD147-hGMCSF was combined with various fusion proteins in (4). FIG. 23-4 shows the rate of emergence of dendritic cells induced in each treatment group.

As shown in FIG. 23-4, use of CD147-hGMCSF alone (indicated by * in the diagram) significantly increased the rate of emergence of dendritic cells compared with the [hGMCSF,hIL4] group. This means that use of the fusion protein CD147-hGMCSF alone was more useful than use of unfused hGMCSF and hIL4. Use of CD147-hGMCSF further combined with 1 type of fusion protein (indicated by †) significantly increased the rate of emergence of dendritic cells compared with the [CD147-hGMCSF] group. This means that use of CD147-hGMCSF further combined with 1 type of fusion protein was more useful than use of the fusion protein CD147-hGMCSF alone.

(5) In the same way as in (1), dendritic cells were induced using MAGEA4-hGMCSF in combination with various fusion proteins. PSA-hGMCSF was combined with various fusion proteins in (1), while MAGEA4-hGMCSF was combined with various fusion proteins in (5). FIG. 23-5 shows the rate of emergence of dendritic cells induced in each treatment group.

As shown in FIG. 23-5, use of MAGEA4-hGMCSF alone (indicated by * in the diagram) significantly increased the rate of emergence of dendritic cells compared with the [hGMCSF,hIL4] group. This means that use of the fusion protein MAGEA4-hGMCSF alone was more useful than use of unfused hGMCSF and hIL4. Use of MAGEA4-hGMCSF further combined with 1 type of fusion protein (indicated by †) significantly increased the rate of emergence of dendritic cells compared with the [MAGEA4-hGMCSF] group. This means that use of MAGEA4-hGMCSF further combined with 1 type of fusion protein was more useful than use of the fusion protein MAGEA4-hGMCSF alone.

(6) In the same way as in (1), dendritic cells were induced using CEA1-hGMCSF in combination with various fusion proteins. PSA-hGMCSF was combined with various fusion proteins in (1), while CEA1-hGMCSF was combined with various fusion proteins in (6). FIG. 23-6 shows the rate of emergence of dendritic cells induced in each treatment group.

As shown in FIG. 23-6, use of CEA1-hGMCSF alone (indicated by * in the diagram) significantly increased the rate of emergence of dendritic cells compared with the [hGMCSF,hIL4] group. This means that use of the fusion protein CEA1-hGMCSF alone was more useful than use of unfused hGMCSF and hIL4. Use of CEA1-hGMCSF further combined with 1 type of fusion protein (indicated by †) significantly increased the rate of emergence of dendritic cells compared with the [CEA1-hGMCSF] group. This means that use of CEA1-hGMCSF further combined with 1 type of fusion protein was more useful than use of the fusion protein CEA1-hGMCSF alone.

(7) In the same way as in (1), dendritic cells were induced using CEA2-hGMCSF in combination with various fusion proteins. PSA-hGMCSF was combined with various fusion proteins in (1), while CEA2-hGMCSF was combined with various fusion proteins in (7). FIG. 23-7 shows the rate of emergence of dendritic cells induced in each treatment group.

As shown in FIG. 23-7, use of CEA2-hGMCSF alone (indicated by * in the diagram) significantly increased the rate of emergence of dendritic cells compared with the [hGMCSF,hIL4] group. This means that use of the fusion protein CEA2-hGMCSF alone was more useful than use of unfused hGMCSF and hIL4. Use of CEA2-hGMCSF further combined with 1 type of fusion protein (indicated by †) significantly increased the rate of emergence of dendritic cells compared with the [CEA2-hGMCSF] group. This means that use of CEA2-hGMCSF further combined with 1 type of fusion protein was more useful than use of the fusion protein CEA2-hGMCSF alone.

The results of Example 12 demonstrated that the combined use of two fusion proteins is more useful in inducing differentiation into dendritic cells than use of a single agent.

### Example 13 Induction of Dendritic Cells Using Various Fusion Proteins and Combinations of Fusion Proteins (Analysis by Flow Cytometry (FCM))

Typical examples of the fusion protein combinations described in Example 11 were used in flow cytometry (FCM) analysis for detecting a dendritic cell surface marker CD86. CD14-positive monocytes of human peripheral blood were cultured for 12 days with 1 type of fusion protein added alone at a concentration of 1 μg/ml or each fusion protein further combined with 1 type of fusion protein (a total of 2 types; all added at a concentration of 1 μg/ml). Dendritic cells (CD86-positive) induced in each treatment group were analyzed by flow cytometry.

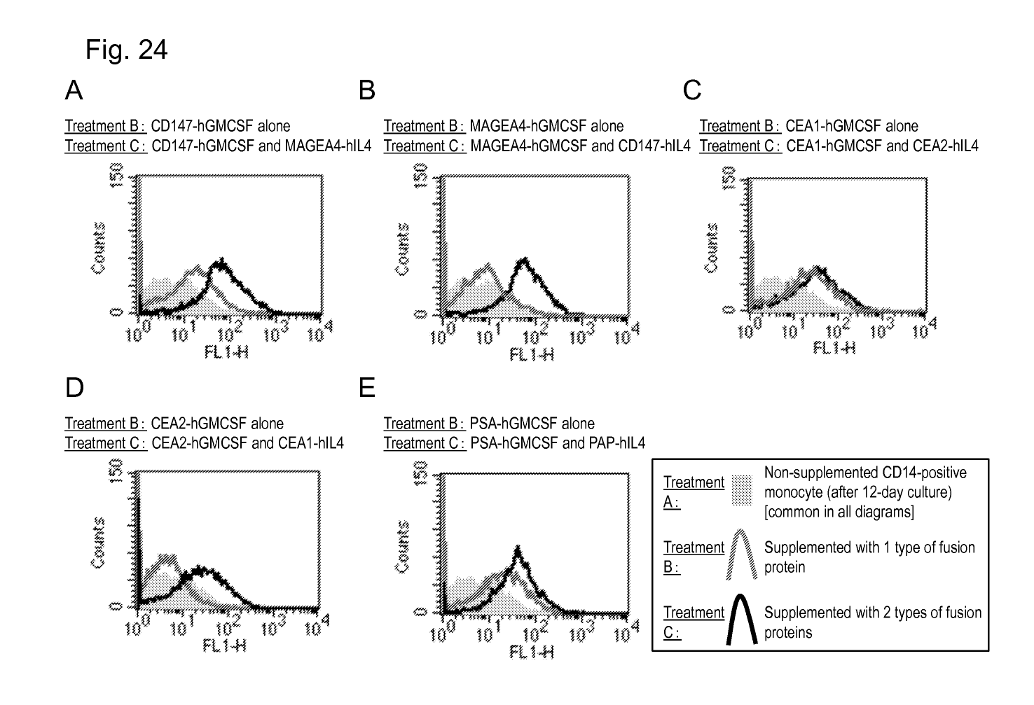

Specifically, the CD14-positive monocytes of human peripheral blood were prepared at a concentration of 1,900,000 cells/well in 6-well plates. Immediately thereafter, various fusion proteins described in FIG. 24 were added thereto. In this state, the cells were cultured for 12 days and stained with an FITC-conjugated anti-human CD86 antibody (BD Pharmingen (Becton, Dickinson and Company); 555657). 5000 cells were analyzed by one run of flow cytometry (FCM) using FACSCalibur flow cytometer (Becton, Dickinson and Company).

The results are shown in FIG. 24. FIGS. 24A to 24E show the results of 5 analyses, respectively. The results of each analysis represent the results of treatment A, treatment B, and treatment C. The results of treatment A were obtained from non-supplemented CD14-positive monocytes (after 12-day culture). The results of treatment B were obtained from CD14-positive monocytes supplemented with 1 type of fusion protein alone. The results of treatment C were obtained from CD14-positive monocytes supplemented with 2 types of fusion proteins in combination. CD147-hGMCSF was used alone in treatment B of FIG. 24A, while CD147-hGMCSF and MAGEA4-hIL4 were used in combination in treatment C thereof. MAGEA4-hGMCSF was used alone in treatment B of FIG. 24B, while MAGEA4-hGMCSF and CD147-hIL4 were used in combination in treatment C thereof. CEA1-hGMCSF was used alone in treatment B of FIG. 24C, while CEA1-hGMCSF and CEA2-hIL4 were used in combination in treatment C thereof. CEA2-hGMCSF was used alone in treatment B of FIG. 24D, while CEA2-hGMCSF and CEA1-hIL4 were used in combination in treatment C thereof. PSA-hGMCSF was used alone in treatment B of FIG. 24E, while PSA-hGMCSF and PAP-hIL4 were used in combination in treatment C thereof

As shown in FIG. 24, a shift to the right side (a larger number of CD86-positive dendritic cells [DCs]) in the graph was significantly observed in the treatment B group or the treatment C group compared with the treatment A group. This means that treatment B or treatment C was useful in inducing differentiation into a larger number of dendritic cells (DCs). In this respect, treatment C using 2 types of fusion proteins in combination was more useful than treatment B using 1 type of fusion protein alone.

### Example 14 Induction of Cytotoxic T Lymphocytes (CD8-Positive), Helper T Lymphocytes (CD4-Positive), or B Lymphocytes (CD19-Positive) Using Various Fusion Proteins and Combinations of Fusion Proteins (Analysis by Flow Cytometry (FCM))

(1) Induction of Cytotoxic T Lymphocytes (CD8-Positive)

Mononuclear cells of human peripheral blood were cultured for 4 days with 1 type of fusion protein added alone at a concentration of 1 μg/ml or each fusion protein further combined with 1 type of fusion protein (a total of 2 types; all added at a concentration of 1 μg/ml). Cytotoxic T lymphocytes (CD8-positive) induced in each treatment group were analyzed by flow cytometry.

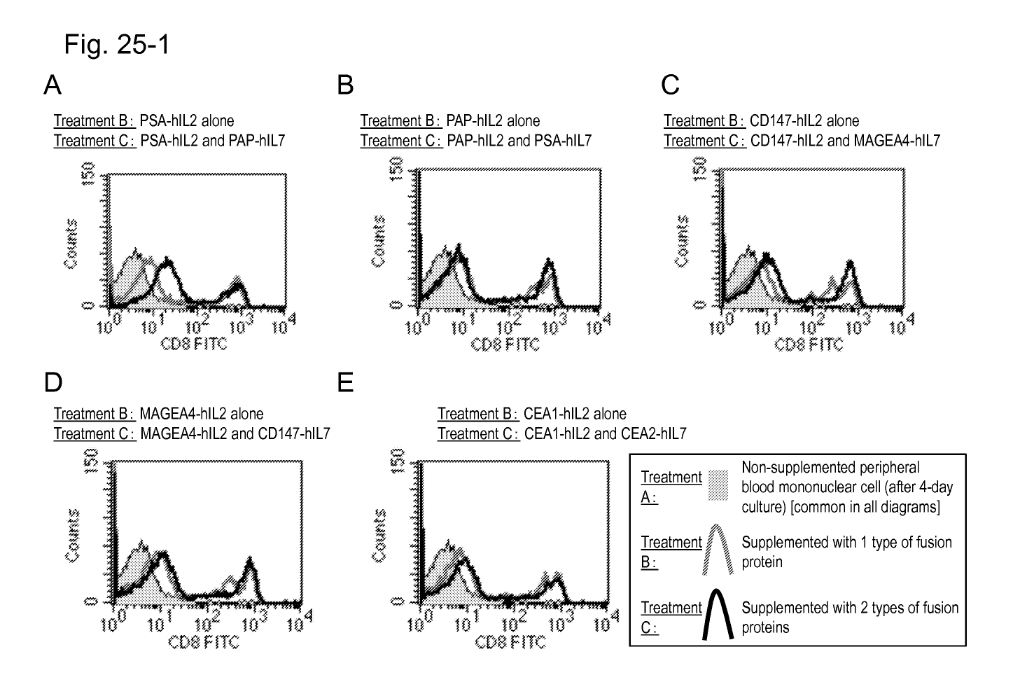

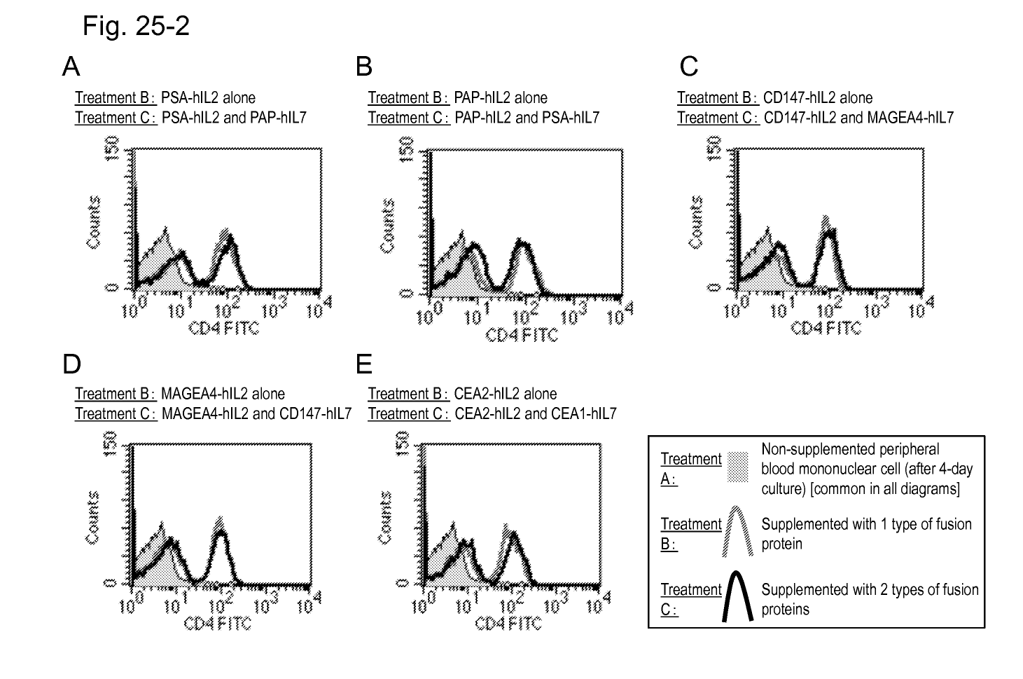

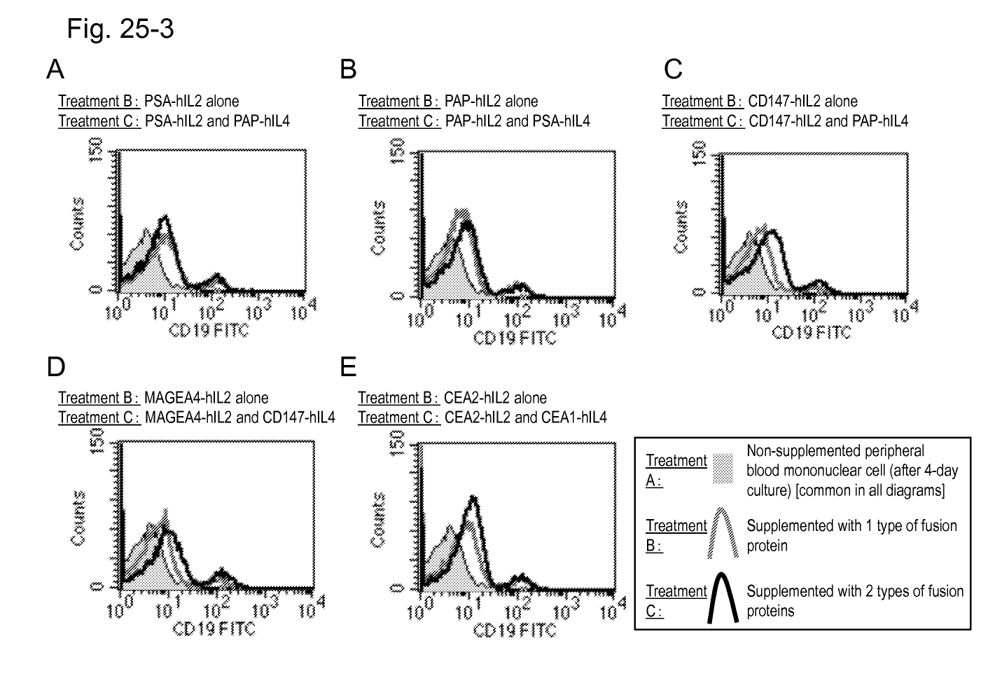

Specifically, the mononuclear cells of human peripheral blood were prepared at a concentration of 750,000 cells/well in 6-well plates. Immediately thereafter, various fusion proteins described in FIG. 25-1 were added thereto. In this state, the cells were cultured for 4 days and stained with an FITC-conjugated anti-human CD8 antibody (BD Pharmingen (Becton, Dickinson and Company); 551347. 20,000 cells were analyzed by one run of flow cytometry (FCM) using FACSCalibur flow cytometer (Becton, Dickinson and Company).

The results are shown in FIG. 25-1. FIGS. 25-1A to 25-1E show the results of 5 analyses, respectively. The results of each analysis represent the results of treatment A, treatment B, and treatment C. The results of treatment A were obtained from non-supplemented peripheral blood mononuclear cells (after 4-day culture). The results of treatment B were obtained from peripheral blood mononuclear cells supplemented with 1 type of fusion protein alone. The results of treatment C were obtained from peripheral blood mononuclear cells supplemented with 2 types of fusion proteins in combination. PSA-hIL2 was used alone in treatment B of FIG. 25-1A, while PSA-hIL2 and PAP-hIL7 were used in combination in treatment C thereof. PAP-hIL2 was used alone in treatment B of FIG. 25-1B, while PAP-IL2 and PSA-hIL7 were used in combination in treatment C thereof. CD147-hIL2 was used alone in treatment B of FIG. 25-1C, while CD147-IL2 and MAGEA4-hIL7 were used in combination in treatment C thereof. MAGEA4-hIL2 was used alone in treatment B of FIG. 25-1D, while MAGEA4-hIL2 and CD147-hIL7 were used in combination in treatment C thereof. CEA1-IL2 was used alone in treatment B of FIG. 25-1E, while CEA1-hIL2 and CEA2-hIL7 were used in combination in treatment C thereof

As shown in FIG. 25-1, a shift to the right side (a larger number of CD8-positive cytotoxic T lymphocytes [CTLs]) in the graph was significantly observed in the treatment B group or the treatment C group compared with the treatment A group. This means that treatment B or treatment C was useful in inducing differentiation into a larger number of CTLs. In this respect, treatment C using 2 types of fusion proteins in combination was more useful than treatment B using 1 type of fusion protein alone.

(2) Induction of Helper T Lymphocytes (CD4-Positive)

Mononuclear cells of human peripheral blood were cultured for 4 days with 1 type of fusion protein added alone at a concentration of 1 μg/ml or each fusion protein further combined with 1 type of fusion protein (a total of 2 types; all added at a concentration of 1 μg/ml). Helper T lymphocytes (CD4-positive) induced in each treatment group were analyzed by flow cytometry.

Specifically, the mononuclear cells of human peripheral blood were prepared at a concentration of 750,000 cells/well in 6-well plates. Immediately thereafter, various fusion proteins described in FIG. 25-2 were added thereto. In this state, the cells were cultured for 4 days and stained with an FITC-conjugated anti-human CD4 antibody (BD Pharmingen (Becton, Dickinson and Company); 555346). 20,000 cells were analyzed by one run of flow cytometry (FCM) using FACSCalibur flow cytometer (Becton, Dickinson and Company).

The results are shown in FIG. 25-2. FIGS. 25-2A to 25-2E show the results of 5 analyses, respectively. The results of each analysis represent the results of treatment A, treatment B, and treatment C. The results of treatment A were obtained from non-supplemented peripheral blood mononuclear cells (after 4-day culture). The results of treatment B were obtained from peripheral blood mononuclear cells supplemented with 1 type of fusion protein alone. The results of treatment C were obtained from peripheral blood mononuclear cells supplemented with 2 types of fusion proteins in combination. PSA-hIL2 was used alone in treatment B of FIG. 25-2A, while PSA-hIL2 and PAP-hIL7 were used in combination in treatment C thereof. PAP-hIL2 was used alone in treatment B of FIG. 25-2B, while PAP-IL2 and PSA-hIL7 were used in combination in treatment C thereof. CD147-hIL2 was used alone in treatment B of FIG. 25-2C, while CD147-IL2 and MAGEA4-hIL7 were used in combination in treatment C thereof. MAGEA4-hIL2 was used alone in treatment B of FIG. 25-2D, while MAGEA4-hIL2 and CD147-hIL7 were used in combination in treatment C thereof. CEA2-IL2 was used alone in treatment B of FIG. 25-2E, while CEA2-hIL2 and CEA1-hIL7 were used in combination in treatment C thereof

As shown in FIG. 25-2, a shift to the right side (a larger number of CD4-positive helper T lymphocytes) in the graph was significantly observed in the treatment B group or the treatment C group compared with the treatment A group. This means that treatment B or treatment C was useful in inducing differentiation into a larger number of helper T lymphocytes L. In this respect, treatment C using 2 types of fusion proteins in combination was more useful than treatment B using 1 type of fusion protein alone.

(3) Induction of B Lymphocytes (CD19-Positive)

Mononuclear cells of human peripheral blood were cultured for 4 days with 1 type of fusion protein added alone at a concentration of 1 μg/ml or each fusion protein further combined with 1 type of fusion protein (a total of 2 types; all added at a concentration of 1 μg/ml). B lymphocytes (CD19-positive) induced in each treatment group were analyzed by flow cytometry.

Specifically, the mononuclear cells of human peripheral blood were prepared at a concentration of 750,000 cells/well in 6-well plates. Immediately thereafter, various fusion proteins described in FIG. 25-3 were added thereto. In this state, the cells were cultured for 4 days and stained with an FITC-conjugated anti-human CD19 antibody (BD Pharmingen (Becton, Dickinson and Company); 555412). 20,000 cells were analyzed by one run of flow cytometry (FCM) using FACSCalibur flow cytometer (Becton, Dickinson and Company).

The results are shown in FIG. 25-3. FIGS. 25-3A to 25-3E show the results of 5 analyses, respectively. The results of each analysis represent the results of treatment A, treatment B, and treatment C. The results of treatment A were obtained from non-supplemented peripheral blood mononuclear cells (after 4-day culture). The results of treatment B were obtained from peripheral blood mononuclear cells supplemented with 1 type of fusion protein alone. The results of treatment C were obtained from peripheral blood mononuclear cells supplemented with 2 types of fusion proteins in combination. PSA-hIL2 was used alone in treatment B of FIG. 25-3A, while PSA-hIL2 and PAP-hIL4 were used in combination in treatment C thereof. PAP-hIL2 was used alone in treatment B of FIG. 25-3B, while PAP-IL2 and PSA-hIL4 were used in combination in treatment C thereof. CD147-hIL2 was used alone in treatment B of FIG. 25-3C, while CD147-IL2 and PAP-hIL4 were used in combination in treatment C thereof. MAGEA4-hIL2 was used alone in treatment B of FIG. 25-3D, while MAGEA4-hIL2 and CD147-hIL4 were used in combination in treatment C thereof. CEA2-hIL2 was used alone in treatment B of FIG. 25-3E, while CEA2-hIL2 and CEA1-hIL4 were used in combination in treatment C thereof

As shown in FIG. 25-3, a shift to the right side (a larger number of CD19-positive B lymphocytes) in the graph was significantly observed in the treatment B group or the treatment C group compared with the treatment A group. This means that treatment B or treatment C was useful in inducing differentiation into a larger number of helper T lymphocytes L. In this respect, treatment C using 2 types of fusion proteins in combination was more useful than treatment B using 1 type of fusion protein alone.

### Example 15 Treatment Experiment of Mouse Models Using Fusion Proteins (Effects on Large Intestine Cancer)

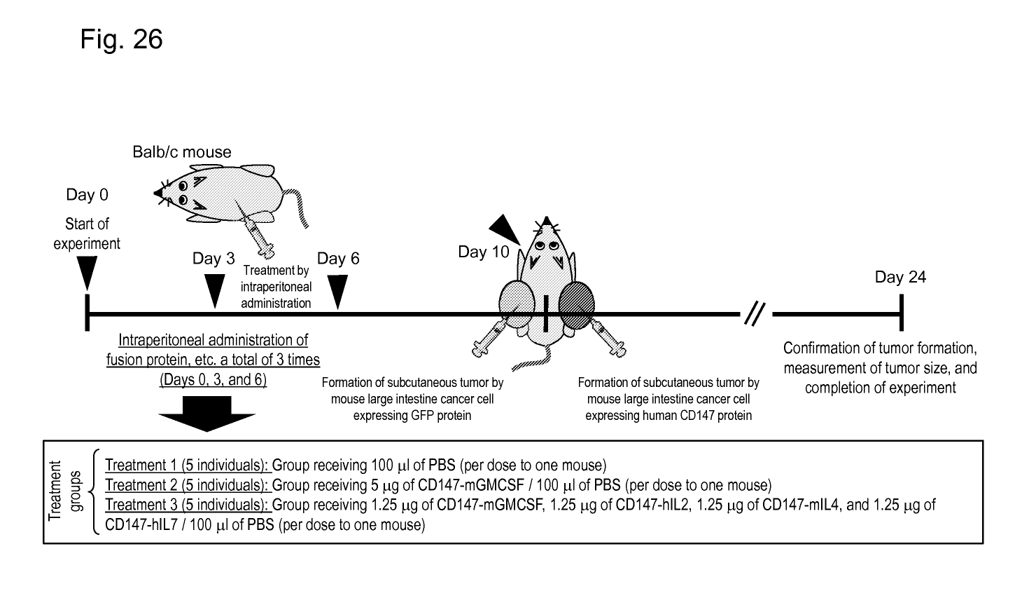

FIG. 26 shows the protocol of the treatment experiment of this Example.

The experiment start date was defined as Day 0. At Days 0, 3, and 6, the fusion protein (combination) was intraperitoneally administered a total of 3 times to each Balb/c mouse (male, 6 to 8 weeks old). In this experiment, treatment 1 to treatment 3 were set for treatment groups. In treatment 1, 100 μl of PBS per dose was administered to each of 5 mice (control). In treatment 2, 5 μg of CD147-mGMCSF/100 μl of PBS per dose was administered to each of 5 mice. In treatment 3, 1.25 μg each of CD147-mGMCSF, CD147-hIL2, CD147-mIL4, and CD147-hIL7/100 μl of PBS per dose was administered to each of 5 mice.

At Day 10, 500,000 mouse CT26 large intestine cancer cells (in 100 μl of PBS) forced to express the GFP protein (left side) and 500,000 CT26 large intestine cancer cells (in 100 μl of PBS) forced to express the human CD147 protein (right side) were subcutaneously transplanted to both thighs of each Balb/c mouse. In each mouse, a subcutaneous tumor was formed by the mouse large intestine cancer cells expressing the GFP protein, while a subcutaneous tumor was formed by the mouse large intestine cancer cells expressing the human CD147 protein. Each gene to be expressed was transferred thereto via plasmid vectors immediately before the transplantation using an electroporation apparatus (NEPA21, NEPA GENE CO., LTD. Chiba, JAPAN). At Day 24, tumor formation was confirmed, and the sizes of the tumors were measured.

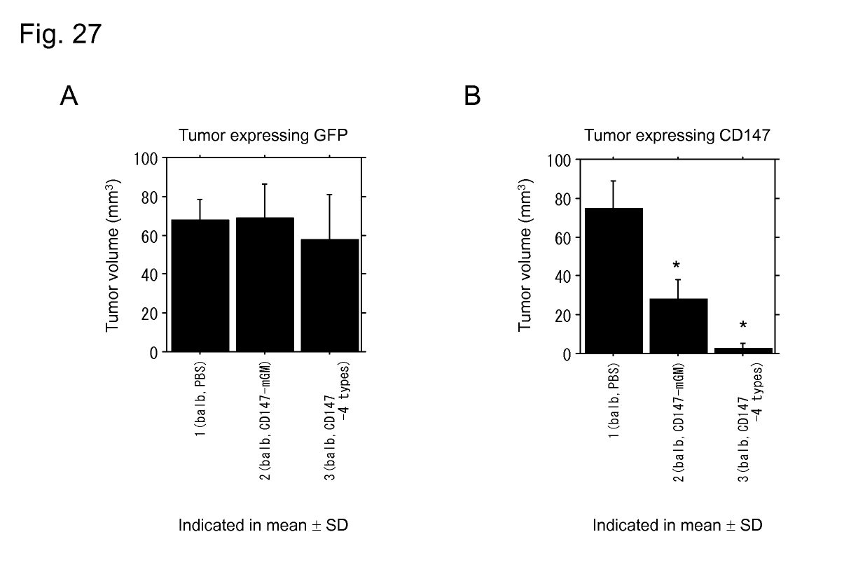

The results are shown in FIG. 27. FIG. 27A shows the size of the tumor expressing GFP. FIG. 27B shows the size of the tumor expressing CD147. As shown in FIG. 27A, the size of the tumor expressing GFP had no significant difference among the treatments. As shown in FIG. 27B, treatment 2 and treatment 3 were able to significantly inhibit tumor growth compared with treatment 1. This means that treatment 2 and treatment 3 were more useful. This result shows that treatment 2 and treatment 3 established in vivo immunity specific for the CD147 protein.

The frequency of mice confirmed to bear the tumor expressing GFP was 5 out of 5 individuals (100%) for treatment 1, 5 out of 5 individuals (100%) for treatment 2, and 5 out of 5 individuals (100%) for treatment 3. As is evident from this result, the tumor incidence had no significant difference among the treatments.

The frequency of mice confirmed to bear the tumor expressing CD147 was 5 out of 5 individuals (100%) for treatment 1, 5 out of 5 individuals (100%) for treatment 2, and 1 out of 5 individuals (20%) for treatment 3. As is evident from this result, treatment 3 was able to significantly (indicated by †) inhibit tumor implantation compared with treatment 2. This means that treatment 3 was more useful.

The results of this Example show that simultaneous use of 4 CD147 fusion proteins can more strongly establish the in vivo immunity specific for the CD147 protein than use of CD147-mGMCSF alone.

The results of this Example demonstrated that the fusion proteins used in this Example are useful in the treatment of large intestine cancer.

Each protein fused with the CD147 protein was considered to be able to cause the effects of the CD147 protein itself (to activate the CD147 protein) through administration into the body of a mouse. However, neither symptoms nor signs indicating adverse reaction or the like were observed in the groups (treatment 2 and treatment 3) receiving the fusion protein(s) containing this CD147 protein component compared with the control group (treatment 1).

### Example 16 Treatment Experiment of Mouse Models Using Fusion Proteins (Effects on Bladder Cancer)

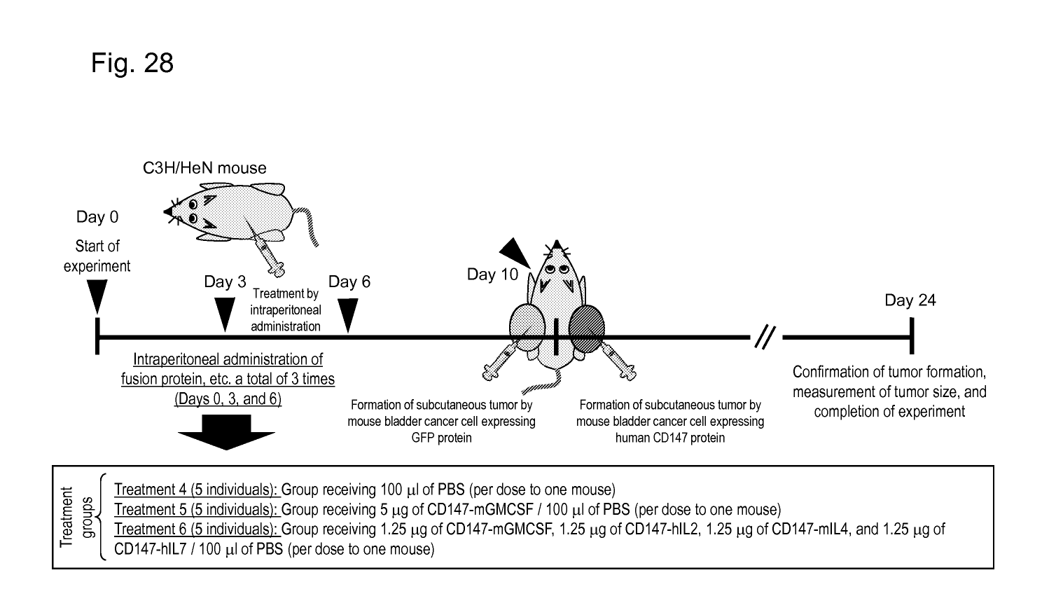

FIG. 28 shows the protocol of the treatment experiment of this Example.

The experiment start date was defined as Day 0. At Days 0, 3, and 6, the fusion protein (combination) was intraperitoneally administered a total of 3 times to each C3H/HeN mouse (male, 6 to 8 weeks old). In this experiment, treatment 4 to treatment 6 were set for treatment groups. In treatment 4, 100 μl of PBS per dose was administered to each of 5 mice (control). In treatment 5, 5 μg of CD147-mGMCSF/100 μl of PBS per dose was administered to each of 5 mice. In treatment 6, 1.25 μg each of CD147-mGMCSF, CD147-hIL2, CD147-mIL4, and CD147-hIL7/100 μl of PBS per dose was administered to each of 5 mice.

At Day 10, 500,000 mouse MBT2 bladder cancer cells (in 100 μl of PBS) forced to express the GFP protein (left side) and 500,000 MBT2 bladder cancer cells (in 100 μl of PBS) forced to express the human CD147 protein (right side) were subcutaneously transplanted to both thighs of each C3H/HeN mouse. In each mouse, a subcutaneous tumor was formed by the mouse bladder cancer cells expressing the GFP protein, while a subcutaneous tumor was formed by the mouse bladder cancer cells expressing the human CD147 protein. Each gene to be expressed was transferred thereto via plasmid vectors immediately before the transplantation using an electroporation apparatus (NEPA21, NEPA GENE CO., LTD. Chiba, JAPAN). At Day 24, tumor formation was confirmed, and the sizes of the tumors were measured.

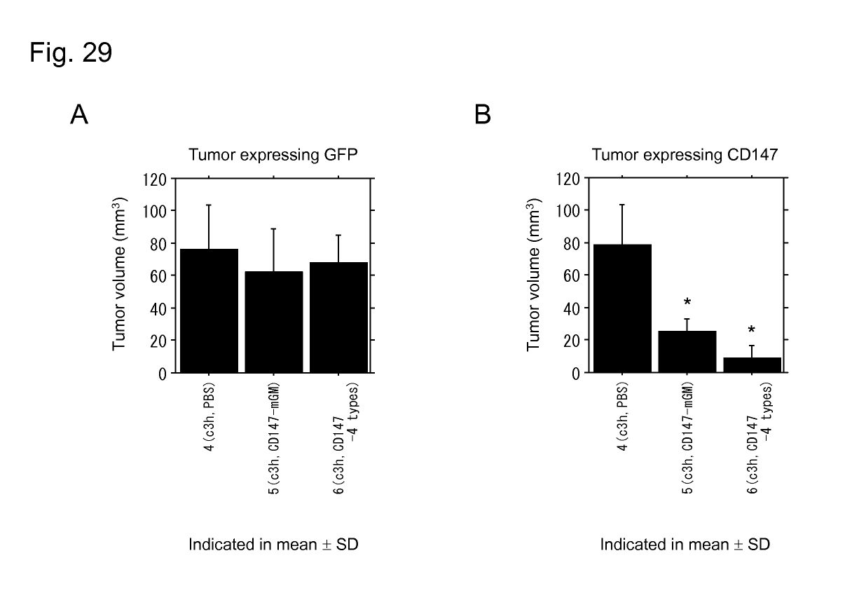

The results are shown in FIG. 29. FIG. 29A shows the size of the tumor expressing GFP. FIG. 29B shows the size of the tumor expressing CD147. As shown in FIG. 29A, the size of the tumor expressing GFP had no significant difference among the treatments. As shown in FIG. 29B, treatment 5 and treatment 6 were able to significantly inhibit tumor growth compared with treatment 4. This means that treatment 5 and treatment 6 were more useful. This result shows that treatment 5 and treatment 6 established in vivo immunity specific for the CD147 protein.

The frequency of mice confirmed to bear the tumor expressing GFP was 5 out of 5 individuals (100%) for treatment 4, 5 out of 5 individuals (100%) for treatment 5, and 5 out of 5 individuals (100%) for treatment 6. As is evident from this result, the tumor incidence had no significant difference among the treatments.

The frequency of mice confirmed to bear the tumor expressing CD147 was 5 out of 5 individuals (100%) for treatment 4, 5 out of 5 individuals (100%) for treatment 5, and 2 out of 5 individuals (40%) for treatment 6. As is evident from this result, treatment 6 was able to significantly (indicated by †) inhibit tumor implantation compared with treatment 5. This means that treatment 6 was more useful.

The results of this Example show that simultaneous use of 4 CD147 fusion proteins can more strongly establish the in vivo immunity specific for the CD147 protein than use of CD147-mGMCSF alone.

The results of this Example demonstrated that the fusion proteins used in this Example are useful in the treatment of bladder cancer.

Each protein fused with the CD147 protein was considered to be able to cause the effects of the CD147 protein itself (to activate the CD147 protein) through administration into the body of a mouse. However, neither symptoms nor signs indicating adverse reaction or the like were observed in the groups (treatment 5 and treatment 6) receiving the fusion protein(s) containing this CD147 protein component compared with the control group (treatment 4).

### Example 17 Treatment Experiment of Mouse Models Using Fusion Proteins (Effects on Lung Cancer)

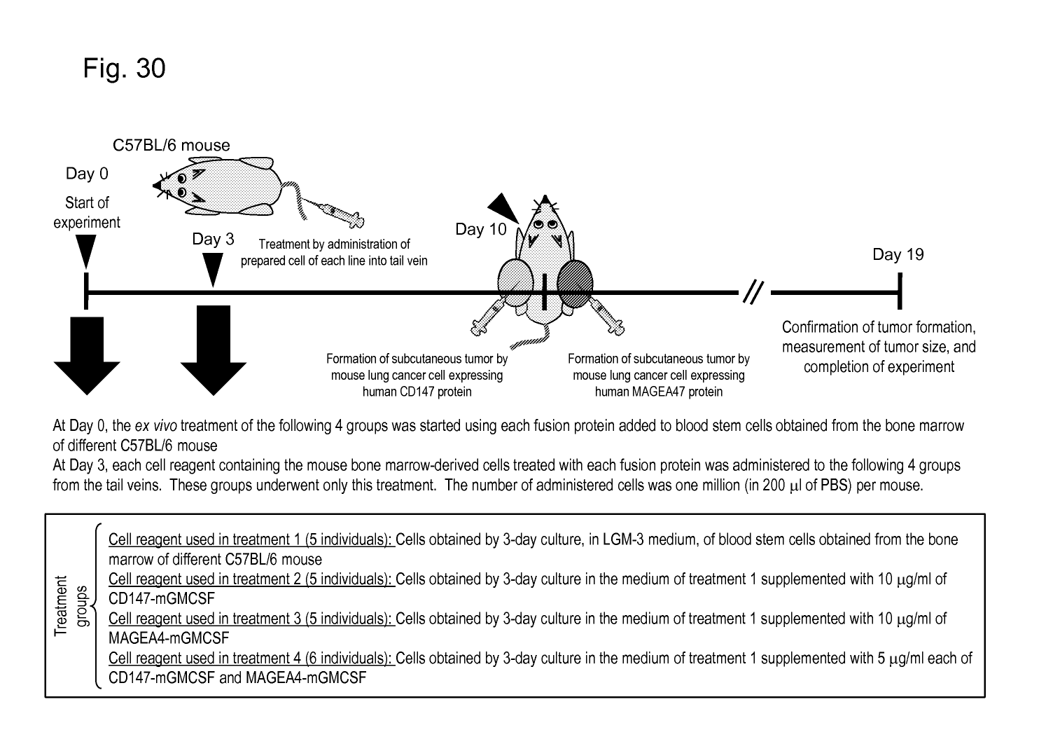

FIG. 30 shows the protocol of the treatment experiment of this Example.

The experiment start date was defined as Day 0. At Day 0, the ex vivo treatment of 4 groups of treatments 1 to 4 was started using each fusion protein (combination) added to blood stem cells obtained from the bone marrow of a different C57BL/6 mouse. At Day 3, each cell reagent containing the mouse bone marrow-derived cells treated with each fusion protein (combination) was administered to the 4 groups of treatments 1 to 4 from the tail vein of each C57BL/6 mouse (male, 6 to 8 weeks old). The cell reagent used in treatment 1 (5 mice) was cells obtained by the 3-day culture, in an LGM-3 medium, of blood stem cells obtained from the bone marrow of a different C57BL/6 mouse. The cell reagent used in treatment 2 (5 mice) was cells obtained by 3-day culture in the medium of treatment 1 supplemented with 10 μg/ml of CD147-mGMCSF. The cell reagent used in treatment 3 (5 mice) was cells obtained by 3-day culture in the medium of treatment 1 supplemented with 10 μg/ml of MAGEA4-mGMCSF. The cell reagent used in treatment 4 (5 mice) was cells obtained by 3-day culture in the medium of treatment 1 supplemented with 5 μg/ml each of CD147-mGMCSF and MAGEA4-mGMCSF. These groups underwent only this treatment. The number of administered cells was one million (in 200 μl of PBS) per mouse.

At Day 10, 1,000,000 mouse LL2 lung cancer cells (in 100 μl of PBS) forced to express the human CD147 protein (left side) and 1,000,000 LL2 lung cancer cells (in 100 μl of PBS) forced to express the human MAGEA4 protein (right side) were subcutaneously transplanted to both thighs of each C57BL/6 mouse. In each mouse, a subcutaneous tumor was formed by the mouse lung cancer cells expressing the human CD147 protein, while a subcutaneous tumor was formed by the mouse lung cancer cells expressing the human MAGEA4 protein. Each gene to be expressed was transferred thereto via plasmid vectors immediately before the transplantation using an electroporation apparatus (NEPA21, NEPA GENE CO., LTD. Chiba, JAPAN). At Day 19, tumor formation was confirmed, and the sizes of the tumors were measured.

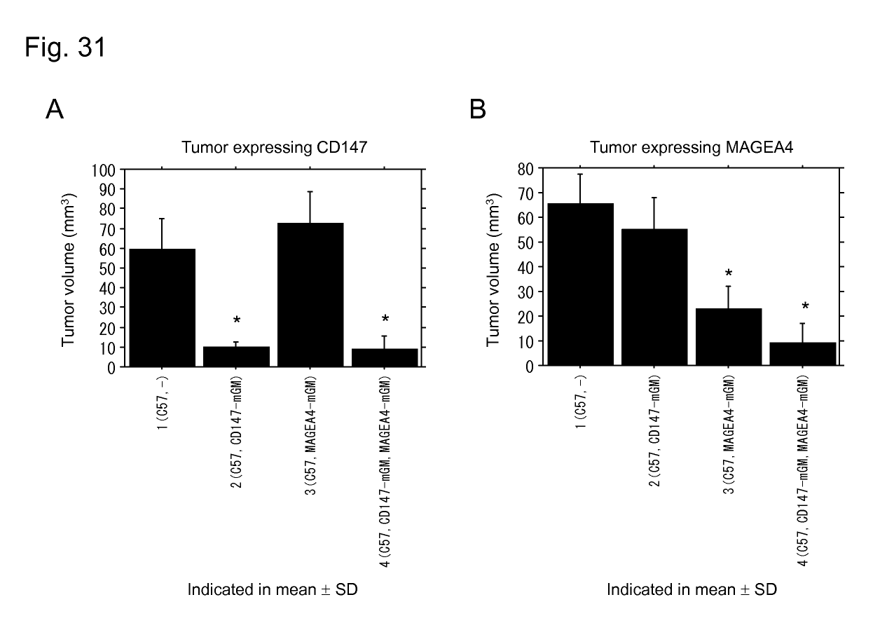

The results are shown in FIG. 31. FIG. 31A shows the size of the tumor expressing CD147. FIG. 31B shows the size of the tumor expressing MAGEA4. As shown in FIG. 31A, treatment 2 and treatment 4 were able to significantly inhibit the growth of the tumor expressing CD147 compared with treatment 1 and treatment 3. This means that treatment 2 and treatment 4 were more useful. This result shows that treatment 2 and treatment 4 established in vivo immunity specific for the CD147 protein. As shown in FIG. 31B, treatment 3 and treatment 4 were able to significantly inhibit tumor growth compared with treatment 1 and treatment 2. This means that treatment 3 and treatment 4 were more useful. This result shows that treatment 3 and treatment 4 established in vivo immunity specific for the MAGEA4 protein.

The frequency of mice confirmed to bear the tumor expressing CD147 was 5 out of 5 individuals (100%) for treatment 1, 5 out of 5 individuals (100%) for treatment 2, 5 out of 5 individuals (100%) for treatment 3, and 2 out of 6 individuals (33.3%) for treatment 4. As is evident from this result, treatment 4 was able to significantly (indicated by †) inhibit tumor implantation compared with treatment 2. This means that treatment 4 was more useful. Simultaneous use of 2 types of fusion proteins CD147-mGMCSF and MAGEA4-mGMCSF can more strongly establish the in vivo immunity against the tumor than use of CD147-mGMCSF alone. This shows that treatment 4 systemically and strongly activated immunity against the tumor and activated in vivo immunity against tumor antigens other than CD147.

The frequency of mice confirmed to bear the tumor expressing MAGEA4 was 5 out of 5 individuals (100%) for treatment 1, 5 out of 5 individuals (100%) for treatment 2, 5 out of 5 individuals (100%) for treatment 3, and 2 out of 6 individuals (33.3%) for treatment 4. As is evident from this result, treatment 4 was able to significantly (indicated by †) inhibit tumor implantation compared with treatment 3. This means that treatment 4 was more useful. Simultaneous use of 2 types of fusion proteins CD147-mGMCSF and MAGEA4-mGMCSF can more strongly establish the in vivo immunity against the tumor than use of MAGEA4-mGMCSF alone. This shows that treatment 4 systemically and strongly activated immunity against the tumor and activated in vivo immunity against tumor antigens other than MAGEA4.

The results of this Example demonstrated that the fusion proteins used in this Example are useful in the treatment of lung cancer.

The results of this Example also show the usefulness of immunotherapy using treated cells obtained by the ex vivo treatment, using fusion proteins, of stem cells capable of differentiating into immunocompetent cells, particularly, the usefulness of therapy using stem cells.

Neither symptoms nor signs indicating adverse reaction or the like were observed in the treatment groups (treatments 1 to 4) of mice receiving the cells treated with the fusion protein(s) compared with the control group (untreated mice).

### Example 18 Treatment Experiment of Mouse Models Using Fusion Proteins (Effects on Stomach Cancer)

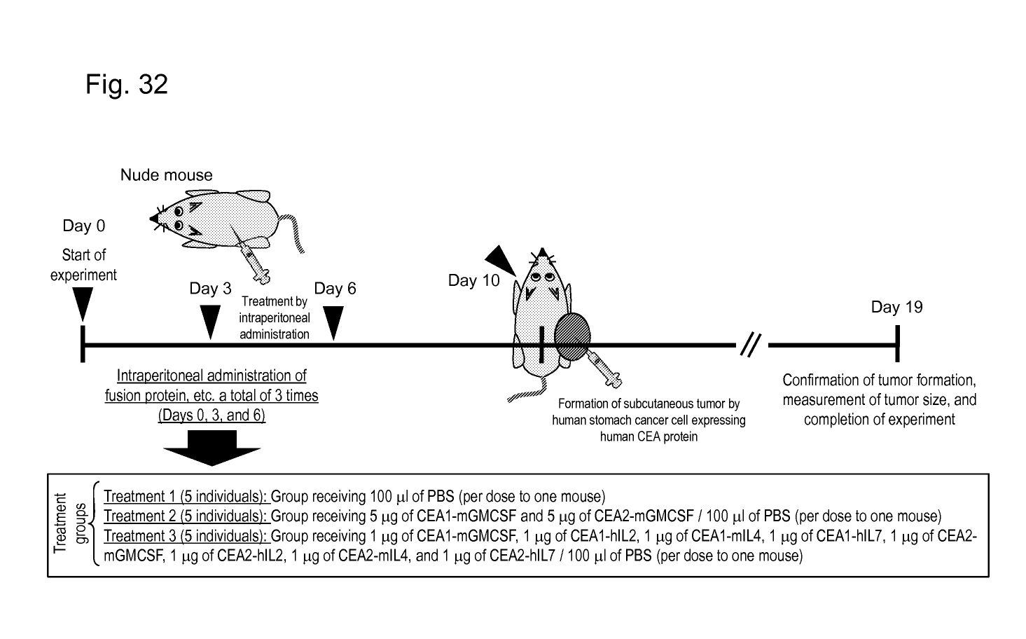

FIG. 32 shows the protocol of the treatment experiment of this Example.

The experiment start date was defined as Day 0. At Days 0, 3, and 6, the fusion proteins were intraperitoneally administered a total of 3 times to each nude mouse (male, 6 to 8 weeks old). In this experiment, treatment 1 to treatment 3 were set for treatment groups. In treatment 1, 100 μl of PBS per dose was administered to each of 5 mice (control). In treatment 2, 5 μg each of CEA1-mGMCSF and CEA2-mGMCSF/100 μl of PBS per dose was administered to each of 5 mice. In treatment 3, 1 μg each of CEA1-mGMCSF, CEA1-hIL2, CEA1-mIL4, CEA1-hIL7, CEA2-mGMCSF, CEA2-hIL2, CEA2-mIL4, and CEA2-hIL7/100 μl of PBS per dose was administered to each of 5 mice.

At Day 10, 1,000,000 human MKN1 stomach cancer cells (in 100 μl of PBS) forced to express the human CEA protein (full length of 668 amino acids) were subcutaneously transplanted to both thighs of each nude mouse. A subcutaneous tumor was formed by the human stomach cancer cells expressing the human CEA protein. Each gene to be expressed was transferred thereto via plasmid vectors immediately before the transplantation using an electroporation apparatus (NEPA21, NEPA GENE CO., LTD. Chiba, JAPAN). At Day 19, tumor formation was confirmed, and the sizes of the tumors were measured.

The frequency of mice confirmed to bear the tumor expressing CEA (full length) was 5 out of 5 individuals (100%) for treatment 1, 0 out of 5 individuals (0%) for treatment 2, and 0 out of 5 individuals (0%) for treatment 3. Treatment 2 and treatment 3 were able to significantly (indicated by †) inhibit tumor implantation compared with treatment 1. This means that treatments 2 and 3 were more useful.

The results of this Example show that treatment 2 and treatment 3 established immunity specific for the CEA protein. The results of this Example demonstrated that the fusion proteins used in this Example are useful in the treatment of stomach cancer. Since nude mice were used in this Example, cytotoxic T cells were absent in vivo. In view of these results together with the results of Examples 13 and 14 using in vitro experiments, the CEA protein-specific immunity established in this Example seems to be based on the activation of humoral immunity via B lymphocytes.

### INDUSTRIAL APPLICABILITY

The fusion proteins of cancer-specific antigens and cytokines according to the present invention can be used as a therapeutic drug for a cancer. Such fusion proteins comprising PSA, PAP, or PSMA as a cancer-specific antigen can be used as a therapeutic drug for prostate cancer. Alternatively, such fusion proteins comprising MAGEA4, CD147, or CEA as a cancer-specific antigen can be used as a therapeutic drug for a wide range of cancer types.

All publications, patents, and patent applications cited herein are incorporated herein by reference in their entirety.

### Free Text for Sequence Listing

SEQ ID NOs: 1 to 13: Synthetic sequences

1.-16. (canceled)

17. A method for preventing or treating a cancer, which comprises administering as active ingredients two or more fusion proteins each comprising a cancer-specific antigen selected from the group consisting of prostate-specific antigen (PSA), prostatic acid phosphatase (PAP), prostate-specific membrane antigen (PSMA), MAGEA4, CD147, and carcinoembryonic antigen (CEA), and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF) to a patient in need thereof.

18. The method according to claim 17, which comprises administering as active ingredients two or more fusion proteins each comprising a cancer-specific antigen selected from the group consisting of prostate-specific antigen (PSA), prostatic acid phosphatase (PAP), and prostate-specific membrane antigen (PSMA), and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF) to a patient in need thereof, wherein the cancer to be prevented or treated is prostate cancer.

19. The method according to claim 18, which comprises administering a fusion protein of prostate-specific antigen (PSA) or prostatic acid phosphatase (PAP) with human or mouse GMCSF, a fusion protein of prostate-specific antigen (PSA) or prostatic acid phosphatase (PAP) with human or mouse IL4, a fusion protein of prostate-specific antigen (PSA) or prostatic acid phosphatase (PAP) with human IL2, and a fusion protein of prostate-specific antigen (PSA) or prostatic acid phosphatase (PAP) with human IL7 as active ingredients to a patient in need thereof.

20. The method according to claim 17, which comprises administering as active ingredients two or more fusion proteins each comprising a cancer-specific antigen selected from the group consisting of MAGEA4, CD147, and carcinoembryonic antigen (CEA), and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF) to a patient in need thereof, wherein the cancer to be prevented or treated is selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma expressing MAGEA4, CD147, or carcinoembryonic antigen (CEA).

21. The method according to claim 20, which comprises administering a fusion protein of CD147 and human or mouse GMCSF, a fusion protein of CD147 and human IL2, a fusion protein of CD147 and human or mouse IL4, and a fusion protein of CD147 and human IL7 as active ingredients to a patient in need thereof, wherein the cancer to be prevented or treated is large intestine cancer or bladder cancer expressing CD147.

22. The method according to claim 20, which comprises administering a fusion protein of CD147 and human or mouse GMCSF and a fusion protein of MAGEA4 and human or mouse GMCSF as active ingredients to a patient in need thereof, wherein the cancer to be prevented or treated is lung cancer expressing CD147 or MAGEA4.

23. The method according to claim 20, which comprises administering a fusion protein of CEA1 and human or mouse GMCSF and a fusion protein of CEA2 and human or mouse GMCSF as active ingredients to a patient in need thereof, wherein the cancer to be prevented or treated is stomach cancer expressing CEA.

24. The method according to claim 20, which comprises administering a fusion protein of CEA1 and human or mouse GMCSF, a fusion protein of CEA1 and human IL2, a fusion protein of CEA1 and human or mouse IL4, a fusion protein of CEA1 and human IL7, a fusion protein of CEA2 and human or mouse GMCSF, a fusion protein of CEA2 and human IL2, a fusion protein of CEA2 and human or mouse IL4, and a fusion protein of CEA2 and human IL7 as active ingredients to a patient in need thereof, wherein the cancer to be prevented or treated is stomach cancer expressing CEA.

25. A method for preparing an immunocompetent cell having antitumor immunity activity, comprising culturing a cell capable of differentiating into an immunocompetent cell in vitro in the presence of two or more fusion proteins each comprising a cancer-specific antigen selected from the group consisting of prostate-specific antigen (PSA), prostatic acid phosphatase (PAP), prostate-specific membrane antigen (PSMA), MAGEA4, CD147, and carcinoembryonic antigen (CEA), and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

26. The method for preparing an immunocompetent cell having antitumor immunity activity according to claim 25, wherein the cancer-specific antigen is prostate-specific antigen (PSA), prostatic acid phosphatase (PAP), or prostate-specific membrane antigen (PSMA), and the cancer is prostate cancer.

27. The method for preparing an immunocompetent cell having antitumor immunity activity according to claim 25, wherein the cancer-specific antigen is a cancer-specific antigen selected from the group consisting of MAGEA4, CD147, and carcinoembryonic antigen (CEA), and the cancer is selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma.

28. The method for preparing an immunocompetent cell having antitumor immunity activity according to claim 25, wherein the cell capable of differentiating into an immunocompetent cell is a mononuclear cell obtained from peripheral blood, bone marrow fluid, or umbilical cord blood, or a stem cell selected from the group consisting of an induced pluripotent stem (iPS) cell, an embryonic stem cell (ES cell), a blood stem cell including a hematopoietic stem cell in the bone marrow, a mesenchymal stem cell, and a tissue-specific stem cell.

29. The method for preparing an immunocompetent cell according to claim 25, wherein the immunocompetent cell is an immunocompetent cell selected from the group consisting of a dendritic cell, a cytotoxic T lymphocyte, a helper T lymphocyte, and a B lymphocyte.

30. A preparation for the treatment of a cancer, comprising as active ingredients two or more vectors each comprising a DNA construct in which any of DNAs encoding 48 types of fusion proteins represented by PSA-hIL2, PSA-hIL4, PSA-hIL7, PSA-hGMCSF, PSA-mIL4, PSA-mGMCSF, PAP-hIL2, PAP-hIL4, PAP-hIL7, PAP-hGMCSF, PAP-mIL4, PAP-mGMCSF, PSMA-hIL2, PSMA-hIL4, PSMA-hIL7, PSMA-hGMCSF, PSMA-mIL4, PSMA-mGMCSF, MAGEA4-hIL2, MAGEA4-hIL4, MAGEA4-hIL7, MAGEA4-hGMCSF, MAGEA4-mIL4, MAGEA4-mGMCSF, CD147-hIL2, CD147-hIL4, CD147-hIL7, CD147-hGMCSF, CD147-mIL4, CD147-mGMCSF, CEA-hIL2, CEA-hIL4, CEA-hIL7, CEA-hGMCSF, CEA-mIL4, CEA-mGMCSF, CEA1-hIL2, CEA1-hIL4, CEA1-hIL7, CEA1-hGMCSF, CEA1-mIL4, CEA1-mGMCSF, CEA2-hIL2, CEA2-hIL4, CEA2-hIL7, CEA2-hGMCSF, CEA2-mIL4, and CEA2-mGMCSF is inserted in a gene insert moiety in a construct according to FIG. 1, FIG. 2, FIG. 3-1, or FIG. 3-2.

31. The preparation for the treatment of a cancer according to claim 30, wherein the preparation is for the treatment of a cancer selected from the group consisting of brain or nerve tumor, skin cancer, stomach cancer, lung cancer, liver cancer, hepatocellular cancer, mouth cancer, blood cancer including lymphoma and leukemia, malignant lymphoma, glioma, melanoma, large intestine cancer, gallbladder cancer, colon cancer, pancreatic cancer, anal or rectal cancer, esophagus cancer, uterus cancer including uterine cervix cancer, ovary cancer, breast cancer, medullary thyroid cancer, adrenal cancer, kidney cancer, renal pelvis and ureter cancer, bladder cancer, prostate cancer, urethral cancer, penis cancer, testicular cancer, osteoma or osteosarcoma, leiomyoma, rhabdomyoma, and mesothelioma.

32. A method for preventing or treating a disease involving CD147, which comprises administering as active ingredients one or two or more fusion proteins each comprising CD147 and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF) to a patient in need thereof.

33. The method according to claim 32, wherein the disease involving CD147 is selected from the group consisting of a lung disease, a malignant disease, an immunity-related disease, a cardiovascular disease, a nervous system disease, a fibrosis, and an infection.

34. A method for preparing a cell usable in the prevention or treatment of a disease involving CD147, comprising culturing a cell capable of differentiating into an immunocompetent cell in vitro in the presence of one or two or more fusion proteins each comprising CD147 and a cytokine selected from the group consisting of human IL2 (hIL2), human IL4 (hIL4), human IL7 (hIL7), human GMCSF (hGMCSF), mouse IL4 (mIL4), and mouse GMCSF (mGMCSF).

35. The method for preparing a cell usable in the prevention or treatment according to claim 34, wherein the disease involving CD147 is selected from the group consisting of a lung disease, a malignant disease, an immunity-related disease, a cardiovascular disease, a nervous system disease, a fibrosis, and an infection.


SEQUENCE LISTING (20 sequences)

SEQ ID NO: 1 - DNA, 5458 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagcattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctggaggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaaccccggaggacccgggggcgcacccgccagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 2 - DNA, 5809 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagcaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaatttcagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatgggattcacaagcagaaagagaaatctaggctccaagggggtgtcctggtcaatgaaatcctcaatcacatgaagagagcaactcagataccaagctacaaaaaacttatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccctagctgtcctctggagaggtttgctgagctggttggccctgtgatccctcaagactggtccacggagtgtatgaccacaaacagccatcaaggtactgaggacagtacagatggaggacccgggggcgcacccgccagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 3 - DNA, 453 bp, Artificial
  Synthetic
agatctgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttagaagaagaactcaaacctctggaggaagtgctaaatttagctcaaagcaaaaactttcacctaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggatccaagcttggtcctggtcatcatcatcatcatcattaatctaga

SEQ ID NO: 4 - DNA, 435 bp, Artificial
  Synthetic
agatctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggatccaagcttggtcctggtcatcatcatcatcatcattaatctaga

SEQ ID NO: 5 - DNA, 510 bp, Artificial
  Synthetic
agatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacggatccaagcttggtcctggtcatcatcatcatcatcattaatctaga

SEQ ID NO: 6 - DNA, 429 bp, Artificial
  Synthetic
agatctcacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagcgggcccggacatcatcatcatcatcattaatctaga

SEQ ID NO: 7 - DNA, 402 bp, Artificial
  Synthetic
agatctcatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcggggcccggacatcatcatcatcatcattaatctaga

SEQ ID NO: 8 - DNA, 414 bp, Artificial
  Synthetic
agatctgcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaagggcccggacatcatcatcatcatcattaatctaga

SEQ ID NO: 9 - DNA, 5280 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattccggggaagatcttcccggggtaccccgaggactagttcgacgccggccaagacagcacagacagattgacctattggggtgtttcgcgagtgtgagagggaagcgccgcggcctgtattactagacctgcccttcgcctggttcgtggcgccttgtgaccccgggcccctgccgcctgcaagtcgaaattgcgctgtgctcctgtgctacggcctgtggctggactgcctgctgctgccctactggctggcaagatcaagctctccctggtggccgcgatcctcgcggatccgcgcccaagcttgggttagctagcccctaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgccgctcgagcggaaaaggccttttgctctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 10 - DNA, 6871 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagcataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggaaaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagccggaggacccgggggcgcacccgccagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 11 - DNA, 5686 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagctcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcaaatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtcggaggacccgggggcagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 12 - DNA, 5290 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagcgctgccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaaggtggactccgacgaccagtggggagagtactcctgcgtcttcctccccgagcccatgggcacggccaacatccagctccacgggcctcccagagtgaaggctgtgaagtcgtcagaacacatcaacgagggggagacggccatgctggtctgcaagtcagagtccgtgccacctgtcactgactgggcctggtacaagatcactgactctgaggacaaggccctcatgaacggctccgagagcaggttcttcgtgagttcctcgcagggccggtcagagctacacattgagaacctgaacatggaggccgaccccggccagtaccggtgcaacggcaccagctccaagggctccgaccaggccatcatcacgctccgcgtgcgcagccacggaggacccgggggcagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 13 - DNA, 6742 bp, Artificial
  Synthetic
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgtgtaaaacgacggccagtttatctagtcagcttgattctagctgatcgtggaccggaaggtgagccagtgagttgattgcagtccagttacgctggagtctgaggctcgtcctgaatgatatacgcgtcggagggttgcgtttgagacgggcgacagatacgcgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattcaccatggggcggcttggggccaccctgctgtgcctgctgctggcggcggcggtccccacggcccccgcgcccgctccgacggcgacctcggctccagtcaagcccggcccggccttaagcaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtccgatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgataggaggacccgggggcagatcttctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgttggcgcgccaaggatcgacgagagcagcgcgactgatcagttctggacgagcgagctgtcgtccggcggccgcgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 14 - DNA, 2004 bp, Homo sapiens
aagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtccgatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgata

SEQ ID NO: 15 - protein, 668 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 16 - DNA, 666 bp, Artificial
  Synthetic
aagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtccgatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaac

SEQ ID NO: 17 - protein, 222 aa, Artificial
  Synthetic
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLN

SEQ ID NO: 18 - DNA, 669 bp, Artificial
  Synthetic
ctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggcc

SEQ ID NO: 19 - protein, 223 aa, Artificial
  Synthetic
LSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQA

SEQ ID NO: 20 - protein, 310 aa, Artificial
  Synthetic
KLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI